(19) United States
(12) Patent Application Publication
(10) Pub. No.: US 2026/0209365 A1
(43) Pub. Date: Jul. 23, 2026

(54) ANTI-COMMON GAMMA CHAIN PROTEINS AND COMPOSITIONS OF MATTER

(71) Applicant: SINOMAB BIOSCIENCE LIMITED, Hong Kong (CN)

(72) Inventors: Chin Wai HUI, Hong Kong (CN); Wai Chung WU, Hong Kong (CN); Carol SHIU, Hong Kong (CN); Tak Keung TONG, Hong Kong (CN); Shui On LEUNG, Hong Kong (CN)

(21) Appl. No.: 19/157,837

(22) PCT Filed: Feb. 20, 2024
(86) PCT No.: PCT/CN2024/077700

§ 371 Date: Aug. 19, 2025

Related U.S. Application Data
(60) Provisional application No. 63/485,980, filed on Feb. 20, 2023.

(51) Int. Cl. C07K 16/28; A61P 37/02

(52) U.S. Cl. CPC C07K 16/2866; A61P 37/02; C07K 2317/24

(57) ABSTRACT

The design and generation of antibodies and antigen-binding fragments (e.g., chimeric and humanized antibodies) that bind specifically to and inhibit the activation of human common gamma chain (γc), a shared signaling receptor subunit used by six γc-cytokines, are described in detail herein. Production method and functional assays to evaluate the efficacies of generated antibodies are also described, along with results suggesting the use of such anti-γc antibodies for treating or preventing autoimmune diseases mediated by γc or γc cytokines, such as GVHD.

### PRIORITY

This application claims the benefit of U.S. Provisional Application No. 63/485,980 filed Feb. 20, 2023, the entire contents of which are hereby incorporated by reference.

### SEQUENCE LISTING

The instant application contains an XML-formatted Sequence Listing that was created on Feb. 12, 2023, is named “SBL-011PCT_SL. xml”, is 227, 306 bytes in size, and hereby incorporated by reference in its entirety.

### FIELD OF THE INVENTION

The present invention relates to the development of antigen binding proteins that bind to γc protein and assays to evaluate the efficacies of those proteins in suppressing γc cytokine induced functions in immune cells and suggest their therapeutic potentials in the treatment of autoimmune diseases.

### BACKGROUND OF THE INVENTION

The common gamma chain (γc) was first discovered as a component of the receptor for interleukin (IL)-2 in 1992. Later, γc was characterized as the share d signaling receptor subunit used by six γc-cytokines: IL-2, IL-4, IL-7, IL-9, IL-15 and IL-21. γc is widely expressed on a majority of immune cells, including B, T, natural killer (NK) and innate lymphoid cells (ILCs). These γc-cytokines bind to their respective receptor subunits which dimerize with γc to induce downstream pathways related to broad pleiotropic actions on the innate and adaptive immune systems. Dimerization of two receptor subunits triggers activation of the Janus kinase-signal transducer and activator of transcription (JAK-STAT) pathway. Autophosphorylation of JAK1 and JAK3 kinases after receptor dimerization can in-turn phosphorylate the STATs, which later dimerize and translocate to the nucleus. Now, STATs act as the transcriptional and epigenetic modulators to genes involved in survival, differentiation, proliferation and immune responses.

The gene for γc expression is located on chromosome Xq13.1. γc is mutated in the patients with X-linked severe combined immunodeficiency (XSCID) [Noguchi et al., Cell., 1993; 73 (1): 147-57.], leading to obvious impairment of both cellular and humoral immunities due to failed development of T cells and NK cells, and abnormal immunoglobulin production due to dysfunction and absence of fully mature B cells.

The role of γc cytokines in the anti-tumor effects is described in different literatures. IL-2, IL-7, IL-15, and IL-21 demonstrate the greatest potential for immunotherapy due to their functions in stimulating proliferation and activation of NK, CD4+ helper and CD8+ cytotoxic T cells [Meazza et al., J Biomed Biotechnol., 2011; 2011: 86 1920; Raeber et al., Immunol Rev., 2018; 283 (1): 176-193.]. The administration of these γc cytokines has been testing as combinational therapy in clinical trials [summarized in Pulliam et al., Immunol Lett., 2016; 169: 61-72.]. Yet, the studies indicate γc cytokines have contradictive effects in hematopoietic cancers and so the use of γc cytokines in anti-cancer treatment is still under controversy.

In contrast, increased level of γc cytokines is consistently observed in human autoimmune conditions and is related to the progression of autoimmune diseases. IL-2 is correlated to progression of asthma [Clinical trial NCT01246414], vitiligo [Ranjkesh et al., Indian J Dermatol., 2021; 66 (4): 366-370.], multiple sclerosis (MS) [Sharief and Thompson J Neurol Neurosurg Psychiatry., 1993; 56 (2): 169-174.] and celiac disease [Tye-Din et al., Aliment Pharmacol Ther., 2019; 50 (8): 901-910.]. IL-4 is correlated to progression of atopic dermatitis (AD) [Chiricozzi et al., Immunotargets Ther., 2020; 9: 151-156], asthma [Pelaia et al., Front Pharmacol., 2022; 13: 851940.], MS [Tahani et al., J Immunoassay Immunochem., 2019; 40 (5): 555-563.], alopecia areata (AA) [El-Latif et al., J Turk Acad Dermatol., 2021; 15 (1): 14-18.], allergic rhinitis (AR) [Liang et al., Front Pharmacol., 2020; 11: 291.] and Rheumatoid Arthritis (RA) [Talaat et al., Cytokine., 2015; 72 (2): 146-53.]. IL-7 is correlated to progression of asthma [Kelly et al., J Immunol., 2009; 182 (3): 1404-1410.], psoriasis [Bonifati et al., Clin Immunol Immunopathol., 1997; 83 (1): 41-4.], AA [Dai et al., Sci Adv., 2021; 7 (14): eabd1866.], Sjogren's syndrome (SS) [Liang et al., Int Immunopharmacol., 2022; 108: 108758.] and type 1 diabetes (TiD) [Monti and Bonifacio Curr Diab Rep., 2014; 14 (9): 518.]. IL-9 is correlated to the progression of AD [Ma et al., Clin Exp Immunol., 2014; 175 (1): 25-31.], asthma [Mahdaviani et al., Acta Biomed., 2021; 92 (3): e2021206.], systemic lupus erythematosus (SLE) and RA [Dantas et al., Dis Markers., 2015; 2015: 519638.]. IL-15 is correlated to progression of RA [Yang et al., Hum Immunol., 2015; 76 (11): 812-8.], psoriasis [Jesus-Gil et al., Exp Dermatol, 2020; 29 (7): 630-638.], vitiligo [Atwa et al., J Cosmet Dermatol., 2021; 20 (8): 2640-2644.], MS [Losy et al., Folia Neuropatho. 1, 2002; 40 (3): 151-3.], AA [Ebrahim et al., Int J Trichology., 2019; 11 (1): 26-30.], celiac disease, SLE and T1D [Abadie and Jabri Immunol Rev., 2014; 260 (1): 221-234.]. IL-21 is correlated to the progression of AD [Mizutani et al., Allergol Int., 2017; 66 (3): 440-444.], RA [Hao et al., BMC Musculoskelet Disord., 2021 Mar. 5; 22 (1): 246., 2021; 66 (3): 440-444.], psoriasis [Wang et al., Am J Transl Res., 2016; 8 (7): 3188-3196.], vitiligo [Custurone et al., Int J Mol Sci., 2021; 22 (21): 11429.], MS [Tzartos et al., Am J Pathol., 2011; 178 (2): 794-802.], AA [Ahmed et al., The Egyptian Journal of Hospital Medicine, 2022; Page 831-836.], celiac disease [Iervasi et al., Autoimmunity., 2020; 53 (4): 225-230.], SS [Kang et al., Arthritis Res Ther., 2011; 13 (5): R179.], T1D [Ferreira et al., Diabetologia., 2015; 58 (4): 781-90.] and SLE [Dolff et al., Arthritis Res Ther., 2011; 13 (5): R157.].

### SUMMARY OF THE INVENTION

The present invention provides the antibodies or antigen binding fragments (monospecific or multispecific) to bind the yc, characterized by one or more of the following: i) bind to purified human yc with the K_{D} lower than 10⁻⁸ M; ii) inhibit the STAT phosphorylation induced by IL-2, IL-4, IL-7, IL-9, IL-15 and IL-21; iii) inhibit the yc cytokine driven functions in the immune cell lines, peripheral blood mononuclear cell (PBMC) and isolated primary immune cell cultures (e.g., proliferation and cytokine release). The antibodies arising from the invention could specifically bind either to the same epitope on yc as a commercially available antibody or antigen binding fragment, or bind to different epitopes on yc as a commercially available antibody or antigen binding fragment thereof which are variants of any of the antibodies or fragments, which are characterized by one or more of the traits set forth above, form part of the present invention.

The present invention also provides the antibodies or antigen binding fragments that could modulate signaling pathways induced by at least one yc-cytokine family m ember, leading to inhibition, amelioration, suppression, treatment or/and prevention of at least one of the autoimmune diseases.

The present invention also provides the DNA sequences for encoding antibodies or antigen binding fragments. The DNA sequences for encoding CDR-H1, CDR-H2 and CDR-H3 of a heavy chain immunoglobulin or variable region thereof are set forth in SEQ ID NO: 1, 17, 33, 49, 65, 81, 97, 113, 129, 145, 161 and/or 177; or a variant thereof; and/or (b) for encoding a light chain immunoglobulin or variable region thereof that comprises CDR-L1, CDR-L2 and CDR-L3 of a light chain immunoglobulin or variable region thereof are set forth in SEQ ID NO: 9, 25, 41, 57, 73, 89, 105, 121, 137, 153, 169 and/or 185; or a variant thereof.

The present invention also provides the antibodies or antigen binding fragments comprising: (a) a heavy chain immunoglobulin or variable region thereof that comprises CDR-H1, CDR-H2 and CDR-H3 of a heavy chain immunoglobulin or variable region thereof that comprises the amino acid sequence set forth in SEQ ID NO: 2, 18, 34, 50, 66, 82, 98, 114, 130, 146, 162 and/or 178; or a variant thereof; and/or (b) a light chain immunoglobulin or variable region thereof that comprises CD R-L1, CDR-L2 and CDR-L3 of a light chain immunoglobulin or variable region thereof that comprises the amino acid sequence set forth in SEQ ID NO: 10, 26, 42, 58, 74, 90, 106, 122, 138, 154, 170 and/or 186; or a variant thereof. In an embodiment of the invention, the antibodies or antigen binding fragments could demonstrate one of the following characteristics: (a) could bind to the same epitope of γc as same as the commercially available yc antibody; (b) could bind to the same epitope of yc as same as the competitor yc antibody; (c) could bind to differential epitopes of yc which are different from the commercially available or competitor yc antibody.

In an embodiment of the invention comprises (a) a heavy chain immuno globulin or variable region thereof comprising an amino acid sequence having at least 90% amino acid sequence identity to the amino acid sequence set forth in SEQ ID NO: 2, 18, 34, 50, 66, 82, 98, 114, 130, 146, 162 and/or 178, and/or (b) a light chain immunoglobulin or variable region thereof comprising an amino acid sequence having at least 90% amino acid sequence identity to the amino acid sequence set forth in SEQ ID NO: 10, 26, 42, 58, 74, 90, 106, 122, 138, 154, 170 and/or 186. For example, in an embodiment of the invention, the antigen-binding protein comprises (a) a heavy chain immunoglobulin or variable region thereof comprising the CDR-H1, CDR-H2 and CDR-H3 of a heavy chain immunoglobulin or variable region thereof comprising an amino acid sequence set forth in SEQ ID NO: 2, 18, 34, 50, 66, 82, 98, 114, 130, 146, 162 and/or 178 and at least 90% amino acid sequence identity to the amino acid sequence set forth in SEQ ID NO: 2, 18, 34, 50, 66, 82, 98, 114, 130, 146, 162 and/or 178, and/or (b) an light chain immunoglobulin or variable region thereof comprising the CDR-L1, CDR-L2 and CDR-L3 of a light cha in immunoglobulin or variable region thereof comprising an amino acid sequence set forth in SEQ ID NO: 10, 26, 42, 58, 74, 90, 106, 122, 138, 154, 170 an d/or 186, and at least 90% amino acid sequence identity to the amino acid sequence set forth in SEQ ID NO: 10, 26, 42, 58, 74, 90, 106, 122, 138, 154, 170 and/or 186.

In an embodiment of the invention, the antibodies or the antigen bin ding protein comprises:
(i) the heavy chain set of CDRs: CDR-H1 comprising the amino acid sequence set forth in SEQ ID NO: 4; CDR-H2 comprising the amino acid sequence set forth in SEQ ID NO: 6; and CDR-H3 comprising the amino acid sequence set forth in SEQ ID NO: 8; and/or CDR-H1 comprising the amino acid sequence set forth in SEQ ID NO: 20; CDR-H2 comprising the amino acid sequence set forth in SEQ ID NO: 22; and CDR-H3 comprising the amino acid sequence set forth in SEQ ID NO: 24; and/or CDR-H1 comprising the amino acid sequence set forth in SEQ ID NO: 36; CDR-H2 comprising the amino acid sequence set forth in SEQ ID NO: 38; and CDR-H3 comprising the amino acid sequence set forth in SEQ ID NO: 40; and/or CDR-H1 comprising the amino acid sequence set forth in SEQ ID NO: 52; CDR-H2 comprising the amino acid sequence set forth in SEQ ID NO: 54; and CDR-H3 comprising the amino acid sequence set forth in SEQ ID NO: 56; and/or CDR-H1 comprising the amino acid sequence set forth in SEQ ID NO: 68; CDR-H2 comprising the amino acid sequence set forth in SEQ ID NO: 70; and CDR-H3 comprising the amino acid sequence set forth in SEQ ID NO: 72; and/or CDR-H1 comprising the amino acid sequence set forth in SEQ ID NO: 84; CDR-H2 comprising the amino acid sequence set forth in SEQ ID NO: 86; and CDR-H3 comprising the amino acid sequence set forth in SEQ ID NO: 88; and/or CDR-H1 comprising the amino acid sequence set forth in SEQ ID NO: 100; CDR-H2 comprising the amino acid sequence set forth in SEQ ID NO: 102; and CDR-H3 comprising the amino acid sequence set forth in SEQ ID NO: 104; and/or CDR-H1 comprising the amino acid sequence set forth in SEQ ID NO: 116; CDR-H2 comprising the amino acid sequence set forth in SEQ ID NO: 118; and CDR-H3 comprising the amino acid sequence set forth in SEQ ID NO: 120; and/or CDR-H1 comprising the amino acid sequence set forth in SEQ ID NO: 132; CDR-H2 comprising the amino acid sequence set forth in SEQ ID NO: 134; and CDR-H3 comprising the amino acid sequence set forth in SEQ ID NO: 136; and/or CDR-H1 comprising the amino acid sequence set forth in SEQ ID NO: 148; CDR-H2 comprising the amino acid sequence set forth in SEQ ID NO: 150; and CDR-H3 comprising the amino acid sequence set forth in SEQ ID NO: 152; and/or CDR-H1 comprising the amino acid sequence set forth in SEQ ID NO: 164; CDR-H2 comprising the amino acid sequence set forth in SEQ ID NO: 166; and CDR-H3 comprising the amino acid sequence set forth in SEQ ID NO: 168; and/or CDR-H1 comprising the amino acid sequence set forth in SEQ ID NO: 180; CDR-H2 comprising the amino acid sequence set forth in SEQ ID NO: 182; and CDR-H3 comprising the amino acid sequence set forth in SEQ ID NO: 184 and/or
(ii) The light chain set of CDRs: CDR-L1 comprising the amino acid sequence set forth in SEQ ID NO: 12; CDR-L2 comprising the amino acid sequence set forth in SEQ ID NO: 14; and CDR-L3 comprising the amino acid sequence set forth in SEQ ID NO: 16; and/or CDR-L1 comprising the amino acid sequence set forth in SEQ ID NO: 28; CDR-L2 comprising the amino acid sequence set forth in SEQ ID NO: 30; and CDR-L3 comprising the amino acid sequence set forth in SEQ ID NO: 32; and/or CDR-L1 comprising the amino acid sequence set forth in SEQ ID NO: 44; CDR-L2 comprising the amino acid sequence set forth in SEQ ID NO: 46; and CDR-L3 comprising the amino acid sequence set forth in SEQ ID NO: 48; and/or CDR-L1 comprising the amino acid sequence set forth in SEQ ID NO: 60; CDR-L2 comprising the amino acid sequence set forth in SEQ ID NO: 62; and CDR-L3 comprising the amino acid sequence set forth in SEQ ID NO: 64; and/or CDR-L1 comprising the amino acid sequence set forth in SEQ ID NO: 76; CDR-L2 comprising the amino acid sequence set forth in SEQ ID NO: 78; and CDR-L3 comprising the amino acid sequence set forth in SEQ ID NO: 80; and/or CDR-L1 comprising the amino acid sequence set forth in SEQ ID NO: 92; CDR-L2 comprising the amino acid sequence set forth in SEQ ID NO: 94; and CDR-L3 comprising the amino acid sequence set forth in SEQ ID NO: 96; and/or CDR-L1 comprising the amino acid sequence set forth in SEQ ID NO: 108; CDR-L2 comprising the amino acid sequence set forth in SEQ ID NO: 110; and CDR-L3 comprising the amino acid sequence set forth in SEQ ID NO: 112; and/or CDR-L1 comprising the amino acid sequence set forth in SEQ ID NO: 124; CDR-L2 comprising the amino acid sequence set forth in SEQ ID NO: 126; and CDR-L3 comprising the amino acid sequence set forth in SEQ ID NO: 128; and/or CDR-L1 comprising the amino acid sequence set forth in SEQ ID NO: 140; CDR-L2 comprising the amino acid sequence set forth in SEQ ID NO: 142; and CDR-L3 comprising the amino acid sequence set forth in SEQ ID NO: 144; and/or CDR-L1 comprising the amino acid sequence set forth in SEQ ID NO: 156; CDR-L2 comprising the amino acid sequence set forth in SEQ ID NO: 158; and CDR-L3 comprising the amino acid sequence set forth in SEQ ID NO: 160; and/or CDR-L1 comprising the amino acid sequence set forth in SEQ ID NO: 172; CDR-L2 comprising the amino acid sequence set forth in SEQ ID NO: 174; and CDR-L3 comprising the amino acid sequence set forth in SEQ ID NO: 176; and/or CDR-L1 comprising the amino acid sequence set forth in SEQ ID NO: 188; CDR-L2 comprising the amino acid sequence set forth in SEQ ID NO: 190; and CDR-L3 comprising the amino acid sequence set forth in SEQ ID NO: 192.

In an embodiment of the invention, the antigen binding protein of the present invention comprises the heavy chain set of CDRs and the light chain set of CDRs as follows:
(i) a heavy chain variable region comprising a CDR-H1 comprising the amino acid sequence set forth in SEQ ID NO: 4; a CDR-H2 comprising the amino acid sequence set forth in SEQ ID NO: 6; and a CDR-H3 comprising the amino acid sequence set forth in SEQ ID NO: 8; and a light chain variable region comprising a CDR-L1 comprising the amino acid sequence set forth in SEQ ID NO: 12; a CDR-L2 comprising the amino acid sequence set forth in SEQ ID NO: 14; and a CDR-L3 comprising the amino acid sequence set forth in SEQ ID NO: 16;
(ii) a heavy chain variable region comprising a CDR-H1 comprising the amino acid sequence set forth in SEQ ID NO: 20; a CDR-H2 comprising the amino acid sequence set forth in SEQ ID NO: 22; and a CDR-H3 comprising the amino acid sequence set forth in SEQ ID NO: 24; and a light chain variable region comprising a CDR-L1 comprising the amino acid sequence set forth in SEQ ID NO: 28; a CDR-L2 comprising the amino acid sequence set forth in SEQ ID NO: 30; and a CDR-L3 comprising the amino acid sequence set forth in SEQ ID NO: 32;
(iii) a heavy chain variable region comprising a CDR-H1 comprising the amino acid sequence set forth in SEQ ID NO: 36; a CDR-H2 comprising the amino acid sequence set forth in SEQ ID NO: 38; and a CDR-H3 comprising the amino acid sequence set forth in SEQ ID NO: 40; and a light chain variable region comprising a CDR-L1 comprising the amino acid sequence set forth in SEQ ID NO: 44; a CDR-L2 comprising the amino acid sequence set forth in SEQ ID NO: 46; and a CDR-L3 comprising the amino acid sequence set forth in SEQ ID NO: 48;
(iv) a heavy chain variable region comprising a CDR-H1 comprising the amino acid sequence set forth in SEQ ID NO: 52; a CDR-H2 comprising the amino acid sequence set forth in SEQ ID NO: 54; and a CDR-H3 comprising the amino acid sequence set forth in SEQ ID NO: 56; and a light chain variable region comprising a CDR-L1 comprising the amino acid sequence set forth in SEQ ID NO: 60; a CDR-L2 comprising the amino acid sequence set forth in SEQ ID NO: 62; and a CDR-L3 comprising the amino acid sequence set forth in SEQ ID NO: 64;
(v) a heavy chain variable region comprising a CDR-H1 comprising the amino acid sequence set forth in SEQ ID NO: 68; a CDR-H2 comprising the amino acid sequence set forth in SEQ ID NO: 70; and a CDR-H3 comprising the amino acid sequence set forth in SEQ ID NO: 72; and a light chain variable region comprising a CDR-L1 comprising the amino acid sequence set forth in SEQ ID NO: 76; a CDR-L2 comprising the amino acid sequence set forth in SEQ ID NO: 78; and a CDR-L3 comprising the amino acid sequence set forth in SEQ ID NO: 80;
(vi) a heavy chain variable region comprising a CDR-H1 comprising the amino acid sequence set forth in SEQ ID NO: 84; a CDR-H2 comprising the amino acid sequence set forth in SEQ ID NO: 86; and a CDR-H3 comprising the amino acid sequence set forth in SEQ ID NO: 88; and a light chain variable region comprising a CDR-L1 comprising the amino acid sequence set forth in SEQ ID NO: 92; a CDR-L2 comprising the amino acid sequence set forth in SEQ ID NO: 94; and a CDR-L3 comprising the amino acid sequence set forth in SEQ ID NO: 96;
(vii) a heavy chain variable region comprising a CDR-H1 comprising the amino acid sequence set forth in SEQ ID NO: 100; a CDR-H2 comprising the amino acid sequence set forth in SEQ ID NO: 102; and a CDR-H3 comprising the amino acid sequence set forth in SEQ ID NO: 104; and a light chain variable region comprising a CDR-L1 comprising the amino acid sequence set forth in SEQ ID NO: 108; a CDR-L2 comprising the amino acid sequence set forth in SEQ ID NO: 110; and a CDR-L3 comprising the amino acid sequence set forth in SEQ ID NO: 112;
(viii) a heavy chain variable region comprising a CDR-H1 comprising the amino acid sequence set forth in SEQ ID NO: 116; a CDR-H2 comprising the amino acid sequence set forth in SEQ ID NO: 118; and a CDR-H3 comprising the amino acid sequence set forth in SEQ ID NO: 120; and a light chain variable region comprising a CDR-L1 comprising the amino acid sequence set forth in SEQ ID NO: 124; a CDR-L2 comprising the amino acid sequence set forth in SEQ ID NO: 126; and a CDR-L3 comprising the amino acid sequence set forth in SEQ ID NO: 128;
(ix) a heavy chain variable region comprising a CDR-H1 comprising the amino acid sequence set forth in SEQ ID NO: 132; a CDR-H2 comprising the amino acid sequence set forth in SEQ ID NO: 134; and a CDR-H3 comprising the amino acid sequence set forth in SEQ ID NO: 136; and a light chain variable region comprising a CDR-L1 comprising the amino acid sequence set forth in SEQ ID NO: 140; a CDR-L2 comprising the amino acid sequence set forth in SEQ ID NO: 142; and a CDR-L3 comprising the amino acid sequence set forth in SEQ ID NO: 144;
(x) a heavy chain variable region comprising a CDR-H1 comprising the amino acid sequence set forth in SEQ ID NO: 148; a CDR-H2 comprising the amino acid sequence set forth in SEQ ID NO: 150; and a CDR-H3 comprising the amino acid sequence set forth in SEQ ID NO: 152; and a light chain variable region comprising a CDR-L1 comprising the amino acid sequence set forth in SEQ ID NO: 156; a CDR-L2 comprising the amino acid sequence set forth in SEQ ID NO: 158; and a CDR-L3 comprising the amino acid sequence set forth in SEQ ID NO: 160;
(xi) a heavy chain variable region comprising a CDR-H1 comprising the amino acid sequence set forth in SEQ ID NO: 164; a CDR-H2 comprising the amino acid sequence set forth in SEQ ID NO: 166; and a CDR-H3 comprising the amino acid sequence set forth in SEQ ID NO: 168; and a light chain variable region comprising a CDR-L1 comprising the amino acid sequence set forth in SEQ ID NO: 172; a CDR-L2 comprising the amino acid sequence set forth in SEQ ID NO: 174; and a CDR-L3 comprising the amino acid sequence set fort h in SEQ ID NO: 176;
(xii) a heavy chain variable region comprising a CDR-H1 comprising the amino acid sequence set forth in SEQ ID NO: 180; a CDR-H2 comprising the amino acid sequence set forth in SEQ ID NO: 182; and a CDR-H3 comprising the amino acid sequence set forth in SEQ ID NO: 184; and a light chain variable region comprising a CDR-L1 comprising the amino acid sequence set forth in SEQ ID NO: 188; a CDR-L2 comprising the amino acid sequence set forth in SEQ ID NO: 190; and a CDR-L3 comprising the amino acid sequence set forth in SEQ ID NO: 192.

Complexes including an antigen-binding protein of the present invention bound to an yc polypeptide or antigenic fragment conjugated to carrier proteins (e.g., murine Fc, 6×His tags (SEQ ID NO: 234)) thereof are also part of the present invention.

The present invention also provides a method for generating and purifying an yc protein that conjugated to the carrier proteins (e.g., murine Fc, 6×His tags (SEQ ID NO: 234)) using mammalian cell expression system (e.g., Expi-CHO cell).

The present invention also provides a method for generating and screening an antigen-binding unit comprising: (a) Animal (e.g., mice) immunization through injection (e.g., subcutaneously, intravenously or intraperitoneally) of purified human γc protein; (b) Generation of phage library through random ligation of a library of antigen-binding single-chain variable fragment (scFv) to the phage vector (e.g., pCANTAB5); (c) Amplification and screening of phage library using purified human γc protein or human cells (e.g., Ramos B cells) expressing γc; (d) Generation and purification of soluble ScFv for γc binding and STAT phosphorylation blocking assays.

The present invention also provides a method for making an antigen-binding protein (e.g., antibody or antigen-binding fragment thereof) or an immunoglobulin chain thereof (e.g., V_{H}, V_{L}, HC or LC) comprising: (a) introducing one or more polynucleotides (or a vector comprising such a polynucleotide) encoding one or more immunoglobulin chains of said antigen-binding protein into a host cell expression system (e.g., a Expi-CHO); (b) culturing the host cell under conditions favorable to expression of the polynucleotides; and (c) isolating the antigen-binding protein or immunoglobulin chain from the host cell and/or medium in which the host cell is grown. An antigen-binding protein or immunoglobulin chain which is a product of such a method also forms part of the present invention. In some embodiments, the antigen-binding protein further comprises a signal peptide (e.g., IL-6 signal peptide) for active secretion from the host cell expression system.

The present invention also provides a polypeptide comprising: (a) a heavy chain immunoglobulin or variable region thereof that comprises CDR-H1, CDR-H2 and CDR-H3 of a heavy chain immunoglobulin or variable region thereof that comprises the amino acid sequence set forth in SEQ ID NO: 2, 18, 34, 50, 66, 82, 98, 1114, 130, 146, 162 and/or 178; or a variant thereof; and/or (b) a light chain immunoglobulin or variable region thereof that comprises CDR-L1, CDR-L2 and CDR-L3 of a light chain immunoglobulin or variable region thereof that comprises the amino acid sequence set forth in SEQ ID NO: 10, 26, 42, 58, 74, 90, 106, 122, 138, 154, 170, and/or 186; or a variant thereof; or (c) the amino acid sequence set forth in a member selected from the group consisting of SEQ ID NO: 1-192, or a variant thereof. The present invention also provides a polynucleotide encoding one or more of such polypeptides set forth in SEQ ID NO: 1, 9, 17, 25, 33, 41, 49, 57, 65, 73, 81, 89, 97, 105, 113, 121, 129, 137, 145, 153, 161, 169, 177, and/or 185; or a vector comprising such a polynucleotide (e.g., a plasmid). In some embodiments, the polypeptide further comprises a signal peptide (e.g., IL-6 signal peptide) for active secretion from the host cell expression system.

The present invention also provides a host cell expression system (e.g., a Expi-CHO) comprising the antigen-binding protein (e.g., antibody or antigen-binding fragment thereof), immunoglobulin chain (e.g., V_{H}, V_{L}, HC or LC), polypeptide, polynucleotide or vector set forth herein.

The present invention describes the construction of humanized anti-γc antibodies using framework patching method (see e.g., U.S. Pat. No. 7,321,026B2). In some embodiments, the framework regions (FRs) from the mouse anti-γc antibodies or/and antigen-binding proteins were replaced by the corresponding FRs of the parent immunoglobulin. In some embodiments, the amino acids inside the humanized FRs were back-mutated to the corresponding amino acids present in the mouse FRs. In some embodiments, framework substitutions were applied to humanized antibodies to increase the binding affinity. In some embodiments, CDR sequences within the humanized antibodies were varied to increase the binding affinity and effector function (e.g., blocking downstream signaling pathway). The resulting humanized antibodies should s how reduced immunogenicity, increased effector function and increase d serum half-life when administrated to the human subjects. The amino acids sequences for humanized variable heavy and light variable chains are set forth in SEQ ID NO: 193-200. The respective humanized heavy chain FRs are set forth in SEQ ID NO: 221 (FR-H1), 222-223 (FR-H2), 224-225 (FR-H3) and 226 (FR-H4); humanized light chain FRs are set forth in SEQ ID NO: 227-228 (FR-L1), 229 (FR-L2), 230-231 (FR-L3) and 232-233 (FR-L4).

The present invention also provides a vessel or injection device (e.g., a vial, syringe, pre-filled syringe or autoinjector) comprising the antigen-binding protein or composition (e.g., pharmaceutical formulation) set forth herein for administration to a subject (e.g., a human or a mouse).

The present invention also provides a method for administering antigen-binding protein or composition set forth herein to a subject (e.g., a human or a mouse) comprising introducing, e.g., injecting (e.g., subcutaneously, intravenously or intraperitoneally), said antigen-binding protein or composition into the body of the subject. The present invention also suggests the therapeutic potential of treating an γc or γc cytokine mediated disease or condition (e.g., graft versus host disease (GVHD)), in a subject in need thereof, comprising administering, e.g., injecting, an effective amount of antigen-binding protein or composition set forth herein.

The present invention also provides a method for blocking STAT phosphorylation in a PBMC (e.g., a T cell, B cell or/and NK cell) or immune cell lines (e.g., mast cell) induced by a γc cytokine (IL-2, IL-4, IL-7, IL-9, IL-15 and/or IL-21); blocking JAK-STAT-mediated (e.g., STAT3, STAT5, STAT6) intracellular signaling pathways and biological functions (e.g., cytokine and chemokine secretion, cell-mediated cytotoxicity, proliferation, cell survival, cell-cell interaction, differentiation, autoimmunity) induced by a γc cytokine (IL-2, IL-4, IL-7, IL-9, IL-15 and/or IL-21); and/or reducing the population of CD45+ immune cells, NK cells, T cells and/or B cells (e.g., excluding neutrophils, eosinophils, granulocytes), in a PBMC culture or a subject (e.g., a human or a mouse), comprising administering to the subject, an effective amount of γc binding protein set forth herein or composition thereof or formulation thereof. In an embodiment of the invention, the subject suffers from an γc or γc cytokine-mediated diseases or conditions, e.g., GVHD, organ transplant rejection, birdshot chorioretinopathy, MS, uveitis, T1D, AD, RA, SLE, asthma, psoriasis, SS, vitiligo, celiac disease, IBD, AA, mast cell mediated diseases, T cell lymphoma, NK cell lymphoma and/or B cell lymphoma.

### BRIEF DESCRIPTION OF THE FIGURES

FIG. 1 demonstrates the binding of purified various clones of anti-γc ScFv to purified human γc proteins in an ELISA assay. Data are expressed in the arbitrary unit as measured by optical density at 450 nm. FIGS. 1A and 1B present the optical density of the various clones of anti-γc scFv to purified human γc proteins in an ELISA assay at 30° C. (FIG. 1A) and 37° C. (FIG. 1B). FIG. 1C presents representative flow cytometry graphs for p-STAT5 blockades by anti-γc scFvs in HPB-ALL.

FIG. 2 demonstrates the example of SDS-PAGE to evaluate the purity and stability of two purified γc binding proteins. More particularly, FIG. 2 presents the results of SDS-PAGE analysis for the purified clones of anti-γc antibodies wherein signal peptides 2 and 3 were selected for the purification of full-length antibodies, wherein HC=heavy immunoglobulin chain; LC=light immunoglobulin chain.

FIG. 3 demonstrates the ELISA binding to purified γc proteins from human (Homo sapiens), cynomolgus (Macaca fascicularis) and chimpanzee (Pan troglodytes) by A) SM-05A and B) SM-05E in ELISA assays. More particularly, FIG. 3A presents the binding curve of SM-05A and FIG. 3B presents the bin ding curve of SM-05E. In each example, the ELISA strip was coated with 1 μg/ml γc protein from each species and antibody was diluted 5-fold each from 10 μg/ml to 0.00064 μg/ml.

FIG. 4 demonstrates the inhibition of (A) IL-2, (B) IL-4, (C) IL-7, (D) IL-9, (E) IL-15 or (F) IL-21 induced STAT phosphorylation in multiple cell lines by selected clones of anti-γc antibody and competitor COMP2022 using flow cytometry analysis. The concentration of cytokines is 10 ng/ml for all groups. All antibodies and inhibitors were administrated at 33 nM (5 μg/ml).

FIG. 5 demonstrates survival of Ramos B cells through a proliferative assay in the presence of anti-IgM, with pre-treatment of IL-4 or IL-21 and various clones of anti-γc antibodies, competitor COMP2022 or JAK3 specific inhibitor ritlecitinib for two days. More particularly, the data presented in FIG. 5 confirms that IL-4 and IL-21 rescue anti-IgM induced deficits in proliferation while the application of anti-γc antibodies (SM-05A, SM-05E, SM-05F) blocks the effects of IL-4 and IL-21. The concentration of IL-4 and IL-21 were 10 ng/ml for all groups. A 11 antibodies and inhibitors were administrated at 33 nM (5 μg/ml). For FIGS. 5A and 5C, *p<0.05 as compared to indicated IgG control group by student t-test, N=5. For FIGS. 5B and 5D, ***p<0.001, **p<0.01, *p<0.05 as compared to anti-IgM+IgG control group by one-way ANOVA, N=5.

FIG. 6 demonstrates cellular damage of Ramos B cells through (A-B) an apoptotic assay and (C-D) western blot in the presence of anti-IgM, with pre-treatment of IL-4 or IL-21 and various clones of anti-γc antibodies, competitor COMP2022 or JAK3 specific inhibitor ritlecitinib for two days. More particularly, the data presented in FIG. 6 confirms that anti-γc antibodies inhibit IL-4 and IL-21 rescues of anti-IgM induced cell death (FIGS. 5A and 5B) and expression of apoptotic/DNA damage markers (FIGS. 5C and 5D). The concentration of IL-4 and IL-21 are 10 ng/ml for all groups. All antibodies and inhibitors were administrated at 33 nM (5 μg/ml). N=5.

FIG. 7 demonstrates suppression of (A) CD23 and (B) PRDM1 expression by selected clones of anti-γc antibody in Ramos B cells after 24-hour treatment. More particularly, the data presented in FIG. 7 confirms that anti-γc antibodies sup pressed CD23 (FIG. 7A) and PRDM1 (FIG. 7B) expression in Ramos B cells after IL-4 or IL-21 stimulation. Concentrations of IL-4 and IL-21 are 10 ng/ml for all groups. All antibodies and inhibitors were administrated at 33 nM (5 μg/ml).

FIG. 8 demonstrates survival of Jurkat T cells through (A) a proliferative assay and (B) p-ERK expression in the presence of IL-9 and various clones of anti-γc antibodies for 24 to 72 hours. More particularly, the data presented in FIG. 8 confirms anti-γc antibodies suppressed IL-9 induced ERK phosphorylation. Con centration of IL-9 is 50 ng/ml for all groups. All antibodies were administrated from 0 to 10 μg/ml (with 5 fold dilution in between).

FIG. 9 demonstrates homeostasis of HPB-ALL T cells by measuring (A) BCL-2 expression, (B) CD127 internalization and (C) CD127 degradation in the presence of IL-7 and various clones of anti-γc antibodies, competitor COMP2022 or ritlecitinib (JAK3 specific inhibitor). More particularly, the data presented in FIG. 9 confirms that anti-γc antibodies suppressed IL-7 induced BCL-2 expression (FIG. 9A), CD127 internalization (FIGS. 9B-9C), and CD127 degradation in HPB-ALL (FIGS. 9D-9E). Concentration of IL-7 is 50 ng/ml for all groups. All antibodies and inhibitors were administrated at 33 nM (5 μg/ml). **p<0.01, *p<0.05 as compared to IgG controls; ##p<0.01, #p<0.05 as compared to IL-7+IgG treated group. Statistical analyses were performed by student t-test. N=3-4.

FIG. 10 demonstrates the establishment of molecular assays to study KHYG-1 homeostasis. Namely, proliferation (FIG. 10A), production of granzyme B (FIG. 10B) and perforin (FIG. 10C), and the active secretion of granzyme A (FIG. 10D), granzyme B (FIG. 10E) and IFN γ (FIG. 10F) are investigated in KHYG-1 culture after cytokine stimulation for 3 days. IL-2 was administrated at 20 or 100 ng/ml, IL-15 was administrated at 20 ng/ml and IL-21 was administrated at 25 ng/ml. All antibodies and inhibitors were administrated at 33 nM (5 μg/ml). ***p<0.001, **p<0.01, *p<0.05 as compared to controls. Statistical analyses were performed by student t-test. N=3.

FIG. 11 compares the suppression of IL-2, IL-15 or IL-21 induced (A-C) proliferation and (D-F) granzyme A secretion in KHYG-1 NK cell after treatment with selected clones of anti-γc antibody, commercially available anti-γc antibody, competitor COMP2022 or ritlecitinib (JAK3 specific inhibitor) for 3 days. More particularly, the data presented in FIG. 11 confirms that anti-γc antibodies suppress IL-2 (FIG. 11A), IL-15 (FIG. 11B) and IL-21 (FIG. 11C) induced KHYG-1 proliferation and secretion of granzyme A (FIGS. 11D-11F) after 3-day incubation. IL-2 and IL-15 were administrated at 20 ng/ml and IL-21 was administrated at 25 ng/ml. All antibodies and inhibitors were administrated at 33 nM (5 g/ml). Rit: ritlecitinib.

FIG. 12 demonstrates the induction of STAT phosphorylation and proliferation by six γc cytokines in PBMC culture. More particularly, the data presented in FIG. 12 includes the representative blot of STAT phosphorylation (FIG. 12A) and the ratio of STAT phosphorylation relative to control (FIG. 12B) and the proliferative index in primary PBMC culture induced by six γc cytokines (FIG. 12C). The concentration of cytokines is 10 ng/ml for all groups. **p<0.01, *p<0.05 as compared to control. Statistical analyses were performed by student t-test. N=3 for PBMC harvested from 3 independent donors.

FIG. 13 demonstrates the inhibition of STAT phosphorylation by selected clones of anti-γc antibodies and competitor COMP2022 in PBMC culture using flow cytometry analyses. The concentration of the six γc cytokines (IL-2, IL-4, IL-7, IL-9, IL-15 and IL-21) is 10 ng/ml for all groups. All antibodies and inhibitors were administrated at 66 nM (10 μg/ml).

FIG. 14 demonstrates the A) proliferative effects of γc cytokines (except IL-9) in the human PBMC culture, B-D) suppression of IL-2, IL-7 and IL-15 induced proliferation in PBMC after treatment with selected clones of anti-γc antibodies and competitor antibody COMP2022 for 3 days. More particularly, the results presented in FIG. 14 demonstrate the proliferative effects of γc cytokines (except IL-9) in the human PBMC culture (FIG. 14A) and the suppression of IL-2 (FIG. 14B), IL-7 (FIG. 14C) and IL-15 (FIG. 14D)—induced proliferation in PBMC after treatment with selected clones of anti-γc antibodies and competitor antibody COMP2022 for 3 days. The concentration of cytokines is 50 ng/ml for all groups. All antibodies and inhibitors were administrated at 33 nM (5 μg/ml). **p<0.01, *p<0.05 as compared to IgG controls. Statistical analyses were performed by One-way ANOVA. N=3-4 for PBMC from independent donors.

FIG. 15 demonstrates A-B) IL-2, IL-15 and IL-21 induced secretion of granzyme A and IFNγ from the PBMC culture as the marker for T and NK cell activation. C-F) suppression of granzyme A and granzyme B secretion by various clones of anti-γc antibodies under IL-2 or IL-15 treatment for three days. More particularly, the results presented in FIG. 15 demonstrate that IL-2, IL-15 and IL-21 induced secretion of granzyme A (FIG. 15A) and IFNγ (FIG. 15B) from the PBMC culture as the marker for T and NK cell activation. FIG. 15C-15F depict the suppression of granzyme A and IFNγ secretion by various clones of anti-γc antibodies and ritlecitinib under IL-2 or IL-15 treatment for three days. IL-2 and IL-15 were administrated at 20 ng/ml and IL-21 was administrated at 25 ng/ml. All antibodies and inhibitors were administrated at 33 nM (5 μg/ml). ***p<0.001, **p<0.01, *p<0.05 as compared to indicated groups. Statistical analyses were performed by student t-test. A-B) N=3-4 for PBMC from independent donors.

FIG. 16 compares the suppression of IL-2 and IL-15 induced expression of FasL on cytotoxic T cells after treatment with SM-05A or ritlecitinib for 3 days. Cytotoxic T cells were gated by CD3+ and CD8+ channels first (FIG. 16A), followed by gating with FasL positive population (FIG. 16B). IL-2 and IL-15 were administrated at 20 ng/ml. All antibodies and inhibitors were administrated at 33 nM (5 μg/ml).

FIG. 17 demonstrates the ELISA binding to purified γc proteins from human (Homo sapiens), cynomolgus (Macaca fascicularis), chimpanzee (Pan troglodytes), common marmoset (Callithrix jacchus), rhesus (Macaca mulatta), mouse (Mus musculus) and rabbit (Oryctolagus cuniculus) by humanized (h) SM-05A (FIG. 17A) and hSM-05E (FIG. 17B) in ELISA assays. The ELISA strip was coated with 1 μg/ml γc protein from each species. Antibody was diluted 5-fold each from 10 μg/ml to 0.00064 μg/ml.

FIG. 18 shows the analytical plots for A-B) hSM-05A and C-D) hSM-05E, 12 days after PBS exchange at RT and 37° C. as performed in size exclusion-high-performance liquid chromatography (SEC-HPLC). More particularly, the data in FIGS. 18A and 18B confirm that hSM-05A maintained its stability and purity (no aggregation) at RT (FIG. 18A) and 37° C. (FIG. 18B). How ever, aggregation was observed in hSM-05E after incubation at RT (FIG. 18C) and 37° C. (FIG. 18D). The blue arrows indicated the sharp peaks representing the monoclonal IgG antibodies. The purity of hSM-05A was around 99.9% while that of hSM-05E was around 80%.

FIG. 19 shows the representative plots for the binding affinities of humanized antibodies to A-B) human γc proteins and C-D) common marmoset, as performed by the Bio-Layer Interferometry (BLI).

FIG. 20 demonstrates the in-vivo binding assay of hSM-05A to γc proteins expressed on the surface of human HEK-293 cells. In brief, hSM-05A was fluorescently labelled with FITC. HEK-293 cells were transfected with the plasmids expressing the full-length γc proteins from different species for 2 days before the incubation with the fluorescent antibodies for flow cytometry analyses. Results demonstrated that hSM-05A could bind to the native γc proteins from humans and common marmoset. All antibodies were administrated at 33 nM (5 μg/ml). C=non-transfected control; Hu: Humans; Rh: Rhesus monkey; Cy: Cynomolgus; Ma: Common marmoset. N=3 for all groups.

FIG. 21 demonstrates the workflow of competition assay between hSM-05A and hSM-05E on the human γc proteins expressed on the surface of Ramos B cells. In brief, hSM-05E was fluorescently labelled with FITC before incubation with unconjugated hSM-05A in the Ramos B cell culture. FIGS. 21A and 21B present representative plots and workflow of a competition assay. The statistical results in FIG. 21C demonstrate that two antibodies could bind to similar epitopes on human γc protein. Statistical analysis was performed by student t-test. N=3 for all groups. All antibodies were administrated at 33 nM (5 μg/ml).

FIG. 22 illustrates the workflows of antibody dependent cellular cytotoxicity (ADCC) and complement dependent cytotoxicity (CDC) for hSM-05A and hSM-05E on human keratinocyte HaCaT cell lines. More particularly, FIG. 22A depicts workflows for investigating ADCC and CDC activities on HaCaT cells. ADCC was determined using PI for evaluation of cell death under flow cytometry analysis, while CDC was studied in 96-well plate via WST-8 proliferative assay. Quantification demonstrated that both hSM-05A and hSM-05E did not induce ADCC (FIG. 22B) and CDC (FIG. 22C) activities on HaCaT cells after incubation with human PBMC or complement cocktail. Statistical analysis was performed by student t-test. N=3 for all groups. All anti bodies were administrated at 33 nM (5 μg/ml).

FIG. 23 illustrates the inhibition of STAT phosphorylation by hSM-05A and hSM-05E in PBMC culture using flow cytometry analyses. More particularly, the results presented in FIG. 23 confirm that inhibition of STAT phosphorylation induced all six γc cytokines-IL-2 (FIG. 23A), IL-7 (FIG. 23B), IL-15 (FIG. 23C), IL-4 (FIG. 23D), and IL-21 (FIG. 23E)—through administration of hSM-05A and hSM-05E to primary T cell culture (IL-2/IL-7/IL-15) and Ramos B cell (IL-4/IL-21). The concentration of cytokines was 50 ng/ml for all groups. Antibodies and inhibitors were diluted 5-fold each from 33 nM to 0.0528 nM.

FIG. 24 demonstrates the efficacies of hSM-05A and hSM-05E to restore the B-cell tolerance checkpoint in anti-IgM stimulated Ramos B cells in the presence of either IL-4 or IL-21. More particularly, the schematic diagram of FIG. 24A proposes that hSM-05A and hSM-05E could block IL-4 induced STAT6 phosphorylation and IL-21 induced STAT3 phosphorylation to push the B cells back to cell death pathway and suppress abnormal survival of autoreactive B cells. As the quantification data presented in FIGS. 24B and 24C confirm, both hSM-05A and hSM-05E can restimulate anti-IgM induced cell death in a comparable level to ritlecitinib as demonstrated in the WST-8 proliferative assay. The percent age of restimulation was calculated as (OD_{anti_IgM/cytokine}−OD_{anti_IgM/cytokine/drug})*100/(OD_{anti-IgM/cytokine}−OD_{anti_IgM}). OD refers to the optical density visualized by the microplate reader after addition of WST-8 reagent. N=3 for ritlecitinib treated groups and N=6 for other groups. All antibodies and inhibitors were administrated at 33 nM (5 μg/ml).

FIG. 25 demonstrates the efficacies of hSM-05A and hSM-05E to suppress IL-7 induced BCL-2 upregulation and CD127 degradation in HPB-ALL T cells. More particularly, the schematic diagram of FIG. 25A proposes that hSM-05A and hSM-05E could block IL-7 induced CD127 degradation and BCL2 upregulation by suppressing JAK3 activation. As the quantification data presented in FIGS. 25B and 25C confirms, both antibodies can suppress two IL-7 induced processes while the effect is stronger in hSM-05A than ritlecitinib treated group. The percentage of suppression was calculated as (P_{IL-7}−P_{IL-7/drug})*100/(P_{IL-7}−P_{Control}). P refers to the percentage of cells showing positive intracellular expression of either BCL-2 or CD127 as determined by flow cytometry. N=3 for all groups. All antibodies and inhibitors were administrated at 33 nM (5 μg/ml).

FIG. 26 illustrates the efficacies of hSM-05A and hSM-05E to suppress IL-9 induced ERK phosphorylation in Jurkat T cells. More particularly, the schematic diagram of FIG. 26A proposes that hSM-05A and hSM-05E could block IL-9 induced ERK phosphorylation and survival by suppressing STAT5 phosphorylation. As the quantification data presented in FIG. 26B confirms, hSM-05A can significantly suppress IL-9 induced protection on ceramide induced cell death. The Western blot results presented in FIG. 26C demonstrate that hSM-05A can inhibit IL-9 induced ERK phosphorylation and quantification confirmed the observation. N=2-3 for all samples. All antibodies were administrated at 33 nM (5 μg/ml). ***p<0.001 *p<0.05 as analyzed by student t-test.

FIG. 27 illustrates the efficacies of hSM-05A and hSM-05E to suppress IL-2 and IL-15 induced proliferation and secretion of cytotoxic factors in human PBMCs. More particularly, the schematic diagram of FIG. 27A proposes that hSM-05A and hSM-05E could block IL-2 and IL-15 induced two phenotypes by suppressing STAT phosphorylation. As the quantification data presented in FIGS. 27B and 27C confirms, both antibodies can suppress IL-2 and IL-15 induced proliferation with a comparable level to ritlecitinib. Similarly, the data presented in FIGS. 27D-27G establishes demonstrates that both antibodies can suppress IL-2 and IL-15 induced IFNγ and granzyme B secretion, while ritlecitinib can totally attenuate those secretions. The percentage of suppression was calculated as (V_{cytokine}−V_{cytokine/drug})*100/(V_{cytokine}−V_{Control}). V refers to OD value recorded by microplate reader from either proliferative (FIG. 27B-27C) or ELISA (FIGS. 27D-27G) experiments. N=3-4 for all groups. All antibodies and inhibitors were administrated at 33 nM (5 μg/ml).

FIG. 28 shows that hSM-05A and hSM-05E could suppress IL-2 and IL-15 driven surface expression of activating NKp46 on human primary NK and cytotoxic T cells. More particularly, FIG. 28A presents representative plots to demonstrate the number of NKp46+NK cells after cytokine and drug treatment. The quantification data presented in FIGS. 28B and 28C confirms a trend for hSM-05A and hSM-05E to reduce IL-2 or IL-15 driven NKp46 upregulation in primary NK cells. Similarly, the quantification data presented in FIGS. 28D and 28E confirms a trend for hSM-05A and hSM-05E to reduce IL-2 or IL-15 driven NKp46 upregulation in primary cytotoxic T cells. N=3 for FIG. 28D-28E. All antibodies and inhibitors were administrated at 33 nM (5 μg/ml).

FIG. 29 illustrates that hSM-05A and hSM-05E could suppress mixed lymphocyte reaction (MLR) in the human PBMC cultures. More particularly, the schematic diagram of FIG. 29A shows that T cell was isolated from donor 1 while T cell was depleted in donor 2. Isolated T cells were labelled with CFS E and then co-cultured with depleted from donor 2. Number of proliferating (CFSE+) T cells could reflect the GvHD condition in the culture through flow cytometry analyses. The quantification data presented in FIGS. 29B-29C demonstrate that both hSM-05A and hSM-05E suppress GvHD driven proliferation of cytotoxic and helper T cells in the culture. N=2 for each group.

FIG. 30 is the summarized diagram showing that how anti-γc antibodies suppressed the JAK/STAT activities to tackle autoreactive T, B and NK cells.

### SUMMARY OF THE TABLES

Table 1 represents heavy chain CDRs in immunoglobulins of the present invention.

Table 2 represents light chain CDRs in immunoglobulins of the present invention.

Table 3 represents heavy and light chain CDRs in each immunoglobulin of the present invention.

Table 4 represents DNA sequences encoding heavy and light chain CDRs in each immunoglobulin of the present invention.

Table 5 represents the immunization procedure of γc protein into the mice for antibody generation.

Table 6 represents the results of monoclonal phage ELISA to 406 positive clones after two to three rounds of panning process.

Table 7 demonstrates the inhibition of IL-15 induced STAT phosphorylation by various clones of anti-γc ScFvs in KHYG-1 NK cell line using flow cytometry analysis. All ScFvs were administrated at 10 μg/ml.

Table 8 demonstrates the inhibition of (A) IL-4 and (B) IL-21 induced STAT phosphorylation by various clones of anti-γc ScFvs in Ramos B cell line using flow cytometry analysis. All ScFvs were administrated at 10 μg/ml.

Table 9 demonstrates the inhibition of IL-7 induced STAT5 phosphorylation by various clones of anti-γc ScFvs in HPB-ALL T cell line using flow cytometry analysis. All ScFvs were administrated at 10 μg/ml.

Table 10 demonstrates the inhibition of IL-9 induced A) STAT3 phosphorylation by various clones of anti-γc ScFvs in Jurkat T cell line using flow cytometry analysis. All ScFvs were administrated at 10 μg/ml.

Table 11 represents binding kinetics of humanized anti-γc antibodies to human, γc protein.

Table 12 represents binding kinetics of humanized anti-γc antibodies to γc proteins from other species.

Table 13 shows the suppression of STAT phosphorylation by hSM-05A and hSM-05E in PBMC culture.

### DETAILED DESCRIPTION OF THE PREFERRED EMBODIMENTS OF THE INVENTION

The present invention provides antibodies and antigen-binding fragments thereof that specifically bind to human and cynomolgus γc and demonstrate modulation of intracellular protein pathways and biological functions, including the blockade of γc cytokine-induced STAT phosphorylation, suppression of cytokine and enzyme secretion, inhibition of hyperactivation and autoreactivity, and apoptosis of autoreactive cells in T, B, NK or/and mast cell cultures. The present invention also provides a clue for the treatment of GVHD, as suggested by the international poster presentation [Azimi et al., American Association for Respiratory Care, 2021], recently published manuscripts [Le Floc'h et al., Sci Transl Med., 2023; 15 (678): eabo0205; Le Floc'h et al., Hemasphere. 2022; 6 (Suppl): 694-695] and clinical trials (Identifier NCT03532958, NCT05589610).

The present invention also provides a method of designing, screening and generating the antibodies and antigen binding fragments through conventional molecular biology, microbiology, and recombinant DNA techniques, phage display techniques and protein purification techniques. Those techniques are fully described in the literature: Sambrook, Fritsch & Maniatis, Molecular Cloning: A Laboratory Manual, Second Edition (1989) Cold Spring Harbor Laboratory Press, Cold Spring Harbor, N.Y.; DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel, et al. (eds.), Current Proto cols in Molecular Biology, John Wiley & Sons, Inc. (1994); M. Hust and TS. Lim, Phage Display Methods and Protocols (2017), Humana Press; P. Meleady, Heterologous Protein Production in CHO Cells Methods and Protocols (2017), Humana Press; R. K. Scope, Protein Purification: Principles and Practice, Second Edition (2013), Springer Science & Business Media.

### Overview

The common gamma chain (γc), also known as P64, CIDX, IMD4, CD132, SCIDX, IL-2RG and SCIDX1, is a shared signaling receptor subunit to several interleukin receptor subunits activated by six γc-cytokines. The receptor subunits include IL-2R, IL-4R, IL-7R, IL-9R, IL-15R and IL-21R.

Receptor dimerization occurs between receptor subunits and γc during the respective γc cytokine induction to trigger downstream JAK-STAT pathway for biological functions. Higher binding affinity and greater stability are triggered during hetero-trimerization of IL-2Rα/IL-2Rβ/γc under IL-2 stimulation and of IL-15Rα/IL-2Rβ/γc under IL-15 stimulation [Stauber et al., Proc Natl Acad Sci USA., 2006; 103 (8): 2788-93; Kobayashi et al., Blood., 2005; 105 (2): 721-7.].

In an embodiment of the invention, human γc is encoded by the nucleo tide sequence set forth under Genbank accession no. NM_000206. In an embodiment of the invention, human γc comprises the amino acid sequence set forth under Genbank accession no. NP_000197.

A whole γc protein comprises of extracellular, transmembrane and intracellular domains [Waickman et al., Cell Mol Life Sci., 2016; 73 (2): 253-269.]. Extracellular domain is divided into 2 type III fibronectin regions (D1 and D2), which could form a disulfide-bond-mediated tertiary structure necessary for interactions with γc cytokine receptor subunits.

γc also possesses a characteristic membrane-proximal WSXWS motif (SEQ ID NO: 235), a conserved amino acid sequence that is present in type I cytokine receptors, involved in receptor activation. The intracellular domain of γc possesses 2 conserved JAK3 binding domains (Box 1 and Box 2 motifs) for JAK1/JAK3 activation and activation downstream signaling events upon receptor dimerization. Degradation motif is located adjacent to the Box 2 motif for proper membrane localization, ligand-mediated internalization and lysosomal degradation.

Six γc cytokines were secreted by a variety of cell types for immune function [Leonard et al., Immunity., 2019; 50 (4): 832-850.]. IL-2 is secreted by both CD4+ helper and CD8+ cytotoxic T cells, activated B cell and dendritic cell (DC) IL-4 is secreted by NK like T cell (NKT), basophil, CD4+ helper T cell eosinophil and mast cell. IL-7 is secreted from stromal cell, intestinal epithelial cell and keratinocyte. IL-9 is secreted from helper T cell 9 (T_{H}9), type 2 innate lymphoid cell (ILC2), mast cell, NKT, IL-9-producing cytotoxic T cell (Tc9), T helper 17 cell (T_{H}17) and regulatory T cell (Treg). IL-15 (or membrane-bound IL-15) is secreted or supplied by DC, monocyte and macrophage. IL-21 is secreted by CD4+ helper T cell, NKT, T follicular helper cell (T_{FH}), T_{H}17 and Gamma delta (γδ) T cell.

Function and Pathology Associated with γc Cytokines

As γc is widely expressed on a majority of immune cells, those γc cytokines contribute to a variety of biological functions, including proliferation, differentiation, maturation, activation and immune functions, in different immune cells as described below [reviewed in Leonard et al., Immunity., 2019; 50 (4): 832-850; Overwijk and Schluns, Clin Immunol., 2009; 132 (2): 153-165].

IL-2 is characterized as the quintessential growth factor for T and NK cells. IL-2 is responsible for T and NK cell proliferation, Treg development, B cell function enhancement, activation-induced cell death (AICD) of T cell, T cell differentiation (T_{H}1, T_{H}2 and T_{H}9), and suppression of T_{H}17 and T_{FH} differentiation. Impaired IL-2 production was reported in SLE patients, leading to decreased Treg population, reduced AICD, hyper-inflammation, and renal Impairment [Lieberman and Tsokos, J Biomed Biotechnol., 2010; 2010: 740619; Shao et al., J Interferon Cytokine Res., 2019; 39 (2): 117-124.]. However, overexpression/increased levels of serum IL-2 induced preferential expansion of Treg [Antony et al., J. Immunol., 2006; 176: 5255-66.], leading to peripheral tolerance of immune functions. Thus, IL-2 is regarded as both a negative and a positive regulator of autoimmunity.

IL-4 is one of the critical cytokines required for B cell differentiation and immunoglobulin class switch in mature B cells. IL-4 is also involved in T_{H}2, T_{H}9 and Treg differentiation/development, alternative macrophage (M2) activation and monocyte-mediated DC differentiation [Hiasa, et al., Blood., 2009; 114 (20): 4517-26.]. The consequences of IL-4 deficiency were poorly studied in the human subject while the animal model of IL-4 deficiency demonstrated increased Treg death, reduced granzyme secretion from Treg [Yang, et al., Front Immunol., 2017; 8: 1508], neural hyperexcitability [Chen et al., Acta Pharm Sin B., 2020; 10 (9): 1634-1645.], resistance to secondary pulmonary P. aeruginosa infection [Song, et al., J Infect Dis., 2015; 211 (10): 1616-27.] and mechanical hypersensitivity [Üçeyler, et al., PLoS One., 2011; 6 (12): e28205.]. Increased serum levels of IL-4 were observed in multiple autoimmune diseases and were believed to play a role in progression of AD and allergic diseases as demonstrated in animal studies [Elbe-Burger et al., J Invest Dermatol., 2002; 118 (5): 767-78; Conde et al., Nat Commun., 2021; 12 (1): 2574.].

IL-7 is discovered as a stromal factor to mediate the development and homeostasis of naïve CD4+ memory T cells and Treg. IL-7 also synergizes with IL-15 to mediate CD8+ memory T cell homeostasis in humans. Loss of IL-7 expression in the transgenic mice leads to profound T cell deficiency [Freeden-Jeffry et al., J Exp Med., 1995; 181: 1519-1526; Peschon et al., J Exp Med., 1994; 180: 1955-1960.]. Abnormal expression of IL-7 also causes T-cell-associated leukemia in mice [Fisher et al., Leukemia., 1993; 2: S66-68.). Elevation of IL-7 leads to preferential expansion of CD4+ and CD8+ T cells, leading to increased risk of allograft rejection [Schreiber et al., Front Immunol., 2019; 10: 742.], development of anti-drug resistant glioma [Cui et al., Cancer Biol Ther., 2012; 13 (7): 496-503.], increased T-cell reactivity to myelin basic protein (MBP) [Traggiai et al., J Neuroimmunol., 2001; 121 (1-2): 111-9.] and amplification of a T_{H}1-driven form of MS [Lee et al., Sci Transl Med., 2011; 3 (93): 93ra68.] as demonstrated in human and mice.

The function of IL-9 is relatively unclear when compared to other γc cytokines. It's believed that IL-9 is involved in T_{H}9 differentiation, anti-tumor effect, mucus production and mast cell homeostasis. Although IL-9 is not required for T cell development and immunoglobulin production, its deficiency leads to enhancement in T_{H}17 response during early pulmonary infection [Li et al., Front Immunol., 2018; 9: 1118.], impaired goblet cell hyperplasia and mastocytosis during pulmonary allergen challenge [Townsend et al., Immunity., 2000; 13 (4): 573-83.] and higher frequencies of Treg, activated CD4+ and CD8+ T cells [Vieyra-Garcia et al., Clin Cancer Res., 2016; 22 (13): 3328-39.]. Increased IL-9 expression could promote ulcerative colitis (UC) by impairing intestinal barrier function and inducing production of pro-inflammatory cytokines by mucosal mononuclear cells [Bird, Nat Rev Immunol., 2014; 14 (7): 432.] and directly induce immune pathology in lung [Temann et al., J Clin Invest., 2002 January; 109 (1): 29-39.] as demonstrated in IL-9 overexpressing mice.

IL-15 is mostly related to the homeostasis of CD8+ cytotoxic T and NK cells. Ligation between transmembrane IL-15 on monocytes/DC and IL-15R triggers development, expansion and activation of CD8+ cytotoxic T and NK cells, as well as inhibiting T_{H}17 differentiation. It is also suspected that IL-15-mediated autocrine mechanisms might be involved in the leukemic transformation of CD4+ T cells. [Azimi et al., Proc Natl Acad Sci USA., 1998; 95: 2452-7; Azimi et al., J. Immunol., 1999; 163: 4064-72; Azimi et al., AIDS Res. Hum. Retroviruses, 2000; 16: 1717-22; Azimi et al., Proc Natl Acad Sci USA., 2001; 98: 14559-64]. IL-15 deficiency directly attenuates NK and CD8+ cytotoxic T cell functions [Suwanai et al., Proc Natl Acad Sci USA., 2010; 107 (20): 9305-10; Kennedy et al., J Exp Med., 2000; 191 (5): 771-80.], leading to 10 times higher susceptibility to breast cancer metastasis in mice [Gillgrass et al., J Immunol., 2014; 193 (12): 6184-91.]. Although overexpression of IL-15 could increase antigen-driven memory CD8+ T cells after microbe exposure [Yajima et al., J Immunol., 2002; 168 (3): 1198-203.], it could promote epithelial damage in patients diagnosed with active celiac disease [Di Sabatino et al., Gut., 2006; 55 (4): 469-477.] and initiate large granular lymphocyte leukemia through chromosomal instability and DNA hypermethylation [Mishra et al., Cancer Cell., 2012; 22 (5): 645-55.].

IL-21 is regarded as a multifunctional regulator of immunity. IL-21 is involved in the enhancement of anti-tumor activities through CD8+ cytotoxic T and NK cells, modulation of B cell apoptotic process, induction of plasma cell differentiation and immunoglobulin production, stimulation of T_{H}17 and T_{FH} differentiation, suppression of T_{H}9 and Treg differentiation, and inhibition of DC maturation and function. IL-21 or/and IL-21R deficiency is indicated in the patients showing severe primary immunodeficiency reminiscent of Common variable immunodeficiency (CVID), leading to impairment in B-cell proliferation, immunoglobulin class-switch, T-cell effector functions and NK cell functions [Kotlarz et al., Curr Opin Pediatr., 2014; 26 (6): 704-12.]. While overexpression of IL-21 could trigger expansion of expansion of hematopoietic progenitor cells in spleen [Ozaki et al., Int J Hematol., 2006; 84 (3): 224-30.], it abnormally drives CD8+ memory T cell accumulation with a concomitant reduction in naive T cell numbers [Allard et al., Eur J Immunol., 2007; 37 (11): 3069-77.], triggers alloimmunity due to reduced Treg function [Petrelli et al., Diabetes., 2011; 60 (12): 3223-3234.] and stimulates the expansion and differentiation of autoreactive B cells in human SLE condition [Wang et al., Nat Commun., 2018; 9 (1): 1758.].

Pathologies Associated with Gene Mutation and Polymorphisms in γc, γc Cytokines and γc Cytokine Receptor Subunits

Hypomorphic mutation in the γc gene is recognized as the cause of XSCID [Lim et al., Allergy Asthma Clin Immunol., 2019; 15: 2.]. More than 300 unique mutations in the γc gene have been identified in the patients. In typical XSCID, there is a complete absence of T and NK cells, and nearly normal or high numbers of functionally deficient B cells, in the patients. Infants with typical XSCID are highly susceptible to bacterial infection and usually die within the first year of life. Milder symptoms are observed in atypical form of XSCID with the atypical phenotypic variants (for example T^{low/-}B⁺NK⁺ and T^{low/-}/B^{low}NK^{+/low/-}). The γc mutation associated with atypical XSCID disrupts the γc configuration and JAK3 binding to the cytoplasmic domain of the γc, leading to abolishment of JAK3 phosphorylation and immunodeficiency in the patients.

In contrast, overexpression of γc is observed in patients with pancreatic ductal adenocarcinoma and gastric cancer [Ayars et al., Oncotarget., 2017; 8 (48): 83370-83383; Wang et al., J Oncol. 2021; 2021: 6670834.]. In-vitro and in-vivo studies also demonstrate that γc expression is directly associated with the pancreatic cancer cel 1 growth and poor prognosis in human gastric cancer.

Gene polymorphism of γc cytokines is highly correlated to the progression of human autoimmune conditions as summarized in the review [Leonard et al., Immunity., 2019; 50 (4): 832-850.]. IL-2 is correlated to MS, T1D and IBD. IL-4 is correlated to asthma and AR. IL-7 is correlated to MS. IL-9 is correlated to AR. IL-15 is correlated to celiac disease [Escudero-Hernández et al., Cytokine., 2017; 99: 73-79.]. IL-21 is correlated to SLE, T1D and IBD.

Gene polymorphism of γc cytokine receptor subunits is also linked to increased risks of autoimmune diseases [Leonard et al., Immunity., 2019; 50 (4): 832-850.]. Gene polymorphism of IL-2RA is related to T1D, IBD, MS; IL-4RA is related to asthma and allergy; IL-7R is related to MS, T1D; IL-9R is related to AR; IL-15RA is related to IBD; IL-21RA is related to SLE. In addition, gene mutation in IL2RA is related to inflammation and autoimmunity in patients with loss-of-function mutations; IL7R is related to T⁻B⁺NK⁺ SCID in patients with loss-of-function mutations; IL21RA is related to defective T and B function and variable dysfunction of NK cells in patients with loss-of-function mutations.

### Current Strategies for Modulating γc Cytokine-Mediated Disorders

As yc cytokines are highly correlated to the progression of autoimmune diseases in humans, therapeutic strategies for modulating or/and treating yc cytokine-mediated diseases by inhibiting yc cytokine family activities have been developed. These methods include the use of ligand which shares the similar structure of yc cytokine to compete with it for the receptor binding site; use of antagonistic peptide which recognizes and blocks the binding site on yc; use of chemical inhibitors targeting JAK-STAT pathway that block the downstream signaling pathway triggered by yc cytokine; and use of specific monoclonal antibodies against yc cytokine to neutralize the targeted cytokine's activity in vivo; use of monoclonal antibodies targeting the individual yc cytokine receptor subunits to selectively inhibit cytokine activity.

IL-2 mutein refers to the genetically engineered IL-2 having a greater binding affinity for IL-2R and a reduced binding affinity for yc as compared to wild-type human IL-2 (hIL-2). One of the examples is H9-RETR, which acts as a competitive inhibitor of IL-2 and IL-15. H9-ERTR is engineered to have higher binding affinity for IL-2Rβ but reduced affinity for yc, which could block IL-2 or/and IL-15 induced CD8+ cytotoxic T cell proliferation and NK cell cytotoxicity in-vitro [Mitra et al., Immunity., 2015; 42 (5): 826-838.]. In in-vivo studies, it could diminish graft-versus-host disease, and block the proliferation of chronic-smoldering adult T cell leukemia T cells. Despite its strong efficacies in the animals, there is only one Phase I/II clinical trial of IL-2 mutein on advanced solid tumor (Identifier: RPCEC00000234). Furthermore, no muteins have been developed for the remaining yc cytokines.

A BNZ-1 is a pegylated peptide with higher binding affinity to yc but diminished affinity to other yc receptor subunits. BNZ-1 is a selective and simultaneous inhibitor with efficacies to block IL-2 and IL-15 mediated leukemia progression [Wang et al., Leukemia., 2019; 33 (5): 1243-1255.]. Its promising effect in blocking IL-2 and IL-15 functions is being tested in Phase II clinical trial of AA (Identifier: NCT03532958) and enters into Phase II/III clinical trial of cutaneous T-cell lymphoma. The variants, namely BNZ-2 and BNZ-3, are later developed to target different combination of yc cytokines.

The use of monoclonal antibodies is regarded as a specific and safer approach to tackle autoimmune diseases. Two monoclonal antibodies were approved by FDA for treatment of autoimmune diseases. Daclizumab could inhibit the binding of IL-2Ra to the IL-2R complex for the treatment of renal allograft rejection and MS. However, it was voluntarily withdrawn from the market as a result of the “complex and evolving benefit/risk profile” of the drug. Daclizumab also ameliorates symptoms in asthma (Identifier: NCT03532958) and partially improves conditions in Uveitis (Identifier: NCT00130637).

Dupilumab is an IL-4Ra targeting monoclonal antibody approved to treat moderate-to-severe asthma and AD. It could inhibit IL-4 signaling via the type 1 receptor (yc) and both IL-4 and IL-13 signaling via the type 2 receptor. Dupilumab is also being tested in multiple clinical trials to treat AA, AR and conjunctivitis. Monoclonal antibodies targeting the remaining yc cytokines/receptor subunits are also investigated in clinical trials. For example, OSE-127 targeting IL-7Ra is currently tested to modulate symptoms in UC and SS. Anti-IL-9 Enokizumab shows beneficial effect in asthma while the endpoint could not be met (Identifier: NCT009 68669). Ordesekimab could directly block IL-15 and is being investigated as the treatment approach of celiac disease, vitiligo, RA and psoriasis. NNC-0114-0006 could target IL-21 and is currently tested in clinical trial of SLE, T1D and Crohns disease.

While cytokine-specific antibodies and blocking peptides demonstrate beneficial effects in animas, multiple cytokines can co-operate to induce a progression of a disease. Thus, antibody approaches involving neutralization of a single yc cytokine or a single yc receptor subunit might not be sufficient to treat autoimmune diseases. Despite the development of IL-2 muteins and blocking peptide, the efficacies are limited to block IL-2 and IL-15 functions without evidence to modulate other yc cytokine activities. Alternative therapeutic strategies may involve the use of small molecule to suppress important downstream proteins activated by yc cytokines, and/or an antibody to target a specific protein receptor implicated in disease pathogenesis whose activity and/or abundance is directly modulated by yc cytokine signaling pathways.

JAK inhibitor is a new class of widely used drugs to suppress downstream JAK1 and JAK3 phosphorylation after yc cytokine stimulation. The use of JAK inhibitor is efficacious to treat several autoimmune diseases due to the blockade of signaling pathways triggered by multiple cytokines simultaneously. It could be classified into five categories: non-selective, JAK1 selective, JAK2 selective, TYK2 selective and JAK3 selective [summarized in Spinell et al., Eur J Immunol., 2021; 51 (7): 1615-1627.]. The first generation of JAK inhibitors (e.g., tofacitinib) was nonselective and approved to treat autoimmune, inflammatory, and hematological conditions. However, it is associated with a wide spectrum of adverse effects (AEs), including serious and opportunistic infections.

The second generation of JAK inhibitors with selectivity to JAK1 (e.g., filgotinib and upadacitinib) and JAK2 (e.g., fedratinib) was developed and approved over the past 10 years. Those selective inhibitors show narrow spectrum of action and provide improved safety profile. However, neutropenias and anemia might occur in fedratinib treated patients likely due to the role of JAK2 in hematopoiesis [Spinell et al., Eur J Immunol., 2021; 51 (7): 1615-1627.]. Also, different class of AEs is observed in filgotinib treated patients. For example, an increase in hemoglobin level was reported as a result of the anti-inflammatory efficacy of selective JAK1 inhibitors combined with the lack of erythropoietin blockade mediated by JAK2 inhibition. JAK1 selective inhibitor also failed to reduce lymphocytes or NK cells' ab solute values probably due to a minor effect of that on the IL-15 signal [as summarized in Biggioggero et al., Drugs Context., 2019; 8: 212595.].

As yc family cytokine requires the common phosphorylation of JAK3 to trigger the downstream signaling pathways, the emergent discovery of JAK3 selective inhibitor is proposed. Ritlecitinib is a covalent inhibitor that binds to the JAK3 catalytic domain and no other JAK family members in which the cysteine residue targeted by this compound is replaced by a serine residue. Ritlecitinib is believed to provide a more specific way to block the yc cytokine signaling as no other receptor molecule recruits JAK3 for signal transduction [Spinell et al., Eur J Immunol., 2021; 51 (7): 1615-1627.]. Ritlecitinib has been demonstrated to show good efficacy in treating autoimmune disease and a more favorable adverse effect profile than other JAK inhibitors, while long-term studies are required to draw the conclusion [Ramírez-Marín and Tosti, Drug Des Devel Ther., 2022; 16: 363-374.]. Ritlecitinib is currently being studied in RA (Identifier: NCT04413617, NCT0 2969044), IBD (Identifier: NCT05636293, NCT02958865, NCT03395184), AA (Identifier: NCT05549934, NCT04006457, NCT03732807, NCT04517864) and vitiligo (Identifier: NCT02974868).

The JAK3 selective approach is promising and provides a hint in the future drug development against autoimmune diseases. The present invention provides an antibody or/and antigen-binding fragment targeting yc, a receptor where the JAK3 conjugates to, as a method to treat GVHD and autoimmune diseases in a subject (e.g., mouse or human) suffered from one of these diseases. The antibody or/and antigen-binding fragment is believed to provide additional advantages (e.g., complete and specific attenuation of yc related downstream effects, lower toxicity or longer half-life) than JAK3 selective inhibitors.

More recently, an anti-yc antibody REGN7257 was enrolled into the Phase I/II clinical trial of aplastic anemia (Identifier: NCT04409080), with the preclinical data suggestive to disease indication including GVHD and MS [Le Floc'h et al., Sci Transl Med., 2023; 15 (678): eabo0205; Le Floc'h et al., Hemasphere. 2022; 6 (Suppl): 694-695]. Those data hints at using the anti-yc strategy to treat multiple auto immune diseases.

γc-Binding Proteins

The present invention provides antigen-binding proteins, such as antibodies (e.g., humanized antibodies, monoclonal antibodies and antibodies conjugated to further therapeutic agent) and antigen binding fragments thereof, which specifically bind to purified yc protein or an antigenic fragment thereof (e.g., the extracellular domain of yc. Antigen-binding proteins binds to the same epitope as a reference antibody or competes for binding to yc with any of the antigen-binding proteins set forth herein, are also part of the present invention.

The present invention also provides any polypeptide or/and antigen-binding proteins that includes an amino acid sequence set forth in SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190 and/or 192 or a variant thereof. In some embodiments, the polypeptide is fused to one or more other polypeptides, e.g., a human Fc (an IgG1, IgG2, IgG3 or IgG4). In some embodiments, the mouse FRs in the anti-γc antibodies (e.g., SM-05A, SM-05E) are substituted by the corresponding FRs present in human antibodies for humanization that includes amino acid sequences for variable heavy chain set forth in SEQ ID NO: 193-194 (underlined amino acids: CDRs) and amino acid sequences for variable light chain set forth in SEQ ID NO: 195-196 (underlined amino acids: CDRs). In some embodiments, the mouse FRs in the anti-yc antibodies (e.g., SM-05A, SM-05E) are substituted by the corresponding FRs present in human antibodies while some of the amino acids are back-mutated to the ones present in mou se with amino acids for variable heavy chain set forth SEQ ID NO: 197-198 (underlined amino acids: CDRs) and variable amino acid sequences for light chain set forth in SEQ ID NO: 199-200 (underlined amino acids: CDRs). The variable heavy and light chains from different antibody clones are interchange able to identify the best humanized antibodies with sustained efficacy and binding affinity to yc proteins.

The present invention also provides any polynucleotides that includes an DNA sequence set forth in SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189 and/or 191, for encoding the polypeptide or/and antigen-binding proteins.

The term “antibody”, and its grammatical equivalents as used herein, refers to an immunoglobulin molecule that recognizes and specifically binds a target, such as a protein, polypeptide, peptide, carbohydrate, polynucleotide, lipid, or a combination of any of the foregoing, through at least one antigen-binding site wherein the antigen-binding site is usually within the variable region of the immunoglobulin molecule. Antibody comprises four polypeptide chains, two heavy chains (HCs) and two light chains (LCs) inter-connected by disulfide bonds (i.e. “full antibody molecules”) (e.g., IgG₁) for example: SM-05A, SM-05B, SM-05C, SM-05D, SM-05E, SM-05F, SM-05G, SM-05H, SM-05I, SM05J, SM-05K, SM-05L. Antibodies also include, but are not limited to, mouse antibodies, rabbit antibodies, camel antibodies, primate antibodies, chimeric antibodies, humanized antibodies, and human antibodies. In an embodiment of the invention, each anti body HC comprises a heavy chain variable region (“HCVR” or “V_{H}”) (e.g., SEQ ID NO: 2, 18, 34, 50, 66, 82, 98, 114, 130, 146, 162 and/or 178, or a variant thereof) and a heavy chain constant region (including domains C_{H}1, C_{H}2 and C_{H}3); and each antibody light chain (LC) comprises a light chain variable region (“LC VR” or “V_{L}”) (e.g., SEQ ID NO: 10, 26, 42, 58, 74, 90, 106, 122, 138, 154, 170 and/or 186, or a variant thereof) and a light chain constant region (C_{L}). The V H and V_{L} regions can be further subdivided into regions of hyper variability, termed complementarity determining regions (CDR), interspersed with regions that are more conserved, termed framework regions (FRs). Each V_{H} and V_{L}, comprises three CDRs and four FRs, arranged from amino-terminus to carboxy-terminus in the following order: FR1, CDR1, FR2, CDR2, FR3, CDR3, FR4. In certain embodiments of the invention, the FRs of the antibody (or antigen binding fragment there of) are identical to the mouse germline sequences or are naturally or artificially modified. In certain embodiments of the invention, the FRs of the antibody (or antigen binding fragment thereof) are identical to the human germline sequences or are naturally or artificially modified.

The terms “antigen-binding portion” or “antigen binding fragment” of an antibody or antigen-binding protein, and the like, as used herein, include any naturally occurring, enzymatically obtainable, synthetic, or genetically engineered polypeptide or glycoprotein that specifically binds an antigen to form a complex. Non-limiting examples of antigen-binding fragments include: (i) Fab fragments; (ii) F (ab′) 2 fragments; (iii) Fd fragments (heavy chain portion of a Fab fragment cleaved with papain); (iv) Fv fragments (a V_{H} or V_{L}); and (v) scFv molecules consisting of a V_{H} and a V_{L} fragments linked with a linker region (e.g., G4S linker (SEQ ID NO: 236)). Other engineered molecules, such as domain-specific antibodies, single domain antibodies, domain-deleted antibodies, chimeric antibodies, CDR-grafted antibodies, bi-specific antibodies, tri-specific antibodies, tetra-specific antibodies, minibodies, nanobodies and small modular immunopharmaceuticals (SMIPs), are also encompassed with in the expression “antigen-binding fragment” as used herein. In an embodiment of the invention, the antigen-binding fragment comprises three or more CDRs of SM-05A, SM-05B, SM-05C, SM-05D, SM-05E, SM-05F, SM-05G, SM-05H, SM-05I, SM05J, SM-05K, SM-05L (e.g., CDR-H1, CDR-H2 and CDR-H3; or CDR-L1, CDR-L2 and CDR-L3).

The term “heavy chain” when used in reference to an antibody refers to a polypeptide chain of about 50-70 kDa, wherein the amino-terminal portion includes a V_{H} of about 120 to 130 or more amino acids and a carboxy-terminal portion that includes a constant region. In some embodiments, the heavy chain constant region is comprised of three domains, CH1, CH2 and CH3, and there is a short flexible hinge region connecting the CH1 and CH2 domains. The constant region can be one of five distinct types, referred to as alpha (a), delta (δ), epsilon (ε), gamma (γ) and mu (μ), based on the amino acid sequence of the heavy chain constant region. The distinct heavy chains differ in size: α, δ and γ contain approximately 450 amino acids, while μ and ε contain approximately 550 amino acids. In an embodiment of the invention, a yc binding protein, e.g., antibody or antigen-binding fragment comprises a heavy chain constant domain to give rise to five well known classes of antibodies, IgA (IgA and IgA2), IgD, IgE, IgG (IgG1, IgG2, IgG3 and IgG4) and IgM, respectively.

The term “light chain” when used in reference to an antibody refers to a polypeptide chain of about 25 kDa, wherein the amino-terminal portion includes a V_{L} of about 100 to about 110 or more amino acids and a carboxy-terminal portion that includes a constant region. The light chain constant region is comprised of one domain, C_{L}. The approximate length of a light chain is 211 to 217 amino acids. There are two distinct types, referred to as kappa (κ) of lambda (λ) based on the amino acid sequence of the constant domains. The present invention includes antigen-binding proteins comprising the variable domains set forth herein (e.g., SM-05A, SM-05B, SM-05C, SM-05D, SM-05E, SM-05F, SM-05G, SM-05H, SM-05I, SM05J, SM-05K, SM-05L), which are linked to a heavy and/or light chain constant domain, e.g., as set forth above.

The term “variable domain” or “variable region” refers to a portion of the light or heavy chains of an antibody that is generally located at the amino-terminal of the light or heavy chain and has a length of about 120 to 130 amino acids in the heavy cha in and about 100 to 110 amino acids in the light chain, and are used in the binding and specificity of each particular antibody for its particular antigen. The variable domains differ extensively in sequence between different antibodies. The variability in sequence is located in the CDRs while the FRs across different antibodies are highly similar. The CDRs of the light and heavy chains are primarily responsible for the interaction of the antibody with antigen. In some embodiments, each V_{H} and V_{L} is composed of three CDRs and four FRs, arranged from amino-terminus to carboxy-terminus in the following order: FR1, CDR1, FR2, CDR2, FR3, CDR3, FR 4. Numbering of amino acid positions used herein is according to the EU Index, as in Kabat et al. (1991) Sequences of proteins of immunological interest. (U.S. Department of Health and Human Services, Washington, D.C.) 5thed.

A CDR refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody V_{L} β-sheet framework. Accordingly, CDRs are variable region sequences interspersed within the framework region sequences. CDR regions are well known to those skilled in the art and have been defined by a variety of methods/systems. These systems and/or definitions have been developed and refined over years and include Kabat, Chothia, IMGT, AbM, and Contact. For example, Kabat defines the regions of most hypervariability within the antibody variable (V) domains [Kabat et al., The Journal of biological chemistry vol. 252, 19, 1977; 6609-16.; Kabat, Advances in protein chemistry vol. 32, 1978; 1-75.]. The Chothia definition is based on the location of the structural loop regions, which defines CDR region sequences as those residues that are not part of the conserved β-sheet framework, and thus are able to adapt different conformations [Chothia, and Lesk, Journal of molecular biology vol. 196, 4, 1987; 901-17]. Both terminologies are well recognized in the art. Additionally, the IMGT system is based on sequence variability and location within the structure of the variable regions. The AbM definition is a compromise between Kabat and Chothia. The Contact definition is based on analyses of the available antibody crystal structures. Software programs (e.g., abYsis) are available and known to those of skill in the art for analysis of antibody sequence and determination of CDRs. The positions of CDRs within a canonical antibody variable domain have been determined by comparison of numerous structures [Al-Lazikani, et al., Journal of molecular biology vol. 273, 4.1997; 927-48; Morea et al., Methods (San Diego, Calif.) vol. 20, 3, 2000; 267-79]. Because the number of residues within a hypervariable region varies in different antibodies, additional residues relative to the canonical positions are conventionally numbered with a, b, c and so forth next to the residue number in the canonical variable domain numbering scheme. Such nomenclature is similarly well known to those skilled in the art.

In an embodiment of the invention, an antigen binding protein of the present invention (e.g., an antibody or antigen-binding fragment thereof) includes a heavy chain immunoglobulin that comprises a V_{H}, including the heavy chain CDRs (CDR-H1, CDR-H2 and CDR-H3). CDRs are defined according to the Kabat (hypervariable) designations in the current invention. The composition of the antigen-binding protein is set forth below in Table 1.

In an embodiment of the invention, an antigen binding protein of the present invention (e.g., an antibody or antigen-binding fragment thereof) includes a light chain immunoglobulin that comprises a V_{L}, including the light chain CDRs (CDR-L1, CDR-L2 and CDR-L3). CDRs are defined according to the Kabat (hypervariable) designations in the current invention. The composition of the antigen-binding protein is set forth below in Table 2.

TABLE 1
Heavy Chain CDRs in Immunoglobulins of the Present Invention

| Antigen-binding fragment | CDR-H1 | CDR-H2 | CDR-H3 |
|---|---|---|---|
| 1 | 4 | 6 | 8 |
| 2 | 20 | 22 | 24 |
| 3 | 36 | 38 | 40 |
| 4 | 52 | 54 | 56 |
| 5 | 68 | 70 | 72 |
| 6 | 84 | 86 | 88 |
| 7 | 100 | 102 | 104 |
| 8 | 116 | 118 | 120 |
| 9 | 132 | 134 | 136 |
| 10 | 148 | 150 | 152 |
| 11 | 164 | 166 | 168 |
| 12 | 180 | 182 | 184 |

CDRs are defined by the nomenclature of Kabat et al., supra
*Numbers correspond to an amino acid sequence set forth in that SEQ ID NO

TABLE 2
Light Chain CDRs in Immunoglobulins of the Present Invention

| Antigen-binding fragment | CDR-L1 | CDR-L2 | CDR-L3 |
|---|---|---|---|
| 1 | 12 | 14 | 16 |
| 2 | 28 | 30 | 32 |
| 3 | 44 | 46 | 48 |
| 4 | 60 | 62 | 64 |
| 5 | 76 | 78 | 80 |
| 6 | 92 | 94 | 96 |
| 7 | 108 | 110 | 112 |
| 8 | 124 | 126 | 128 |
| 9 | 140 | 142 | 144 |
| 10 | 156 | 158 | 160 |
| 11 | 172 | 174 | 176 |
| 12 | 188 | 190 | 192 |

CDRs are defined by the nomenclature of Kabat et al., supra
*Numbers correspond to an amino acid sequence set forth in that SEQ ID NO

In an embodiment of the invention, an antigen-binding protein of the present invention (e.g., an antibody or antigen-binding fragment thereof) includes a heavy and light chain immunoglobulin that comprises a V_{H} (e.g., a HC) and a V_{L} (e.g., a LC), respectively, including the combination of heavy and light chain CDRs (CDR-H1, CDR-H2 and CDR-H3; and CDR-L1, CDR-L2 and CDR-L3) set forth below in Table 3.

TABLE 3
Heavy and Light chain CDRs in Each Immunoglobulins
of the Present Invention

| | CDR- | CDR- | CDR- | CDR- | CDR- | CDR- |
|---|---|---|---|---|---|---|
| Name | H1 | H2 | H3 | L1 | L2 | L3 |
| SM-05A | 4 | 6 | 8 | 12 | 14 | 16 |
| SM-05B | 20 | 22 | 24 | 28 | 30 | 32 |
| SM-05C | 36 | 38 | 40 | 44 | 46 | 48 |
| SM-05D | 52 | 54 | 56 | 60 | 62 | 64 |
| SM-05E | 68 | 70 | 72 | 76 | 78 | 80 |
| SM-05F | 84 | 86 | 88 | 92 | 94 | 96 |
| SM-05G | 100 | 102 | 104 | 108 | 110 | 112 |
| SM-05H | 116 | 118 | 120 | 124 | 126 | 128 |
| SM-05I | 132 | 134 | 136 | 140 | 142 | 144 |
| SM-05J | 148 | 150 | 152 | 156 | 158 | 160 |
| SM-05K | 164 | 166 | 168 | 172 | 174 | 176 |
| SM-05L | 180 | 182 | 184 | 188 | 190 | 192 |

CDRs are defined by the nomenclature of Kabat et al., supra
*Numbers correspond to an amino acid sequence set forth in that SEQ ID NO

The present invention includes an antigen-binding protein (e.g., an antibody or antigen-binding fragment thereof) comprising polypeptide pairs that comprise the following V_{H} and V_{L}, amino acid sequences:
(i) SEQ ID NO: 2 and SEQ ID NO: 10;
(ii) SEQ ID NO: 18 and SEQ ID NO: 26;
(iii) SEQ ID NO: 34 and SEQ ID NO: 42;
(iv) SEQ ID NO: 50 and SEQ ID NO: 58;
(v) SEQ ID NO: 66 and SEQ ID NO: 74;
(vi) SEQ ID NO: 82 and SEQ ID NO: 90;
(vii) SEQ ID NO: 98 and SEQ ID NO: 106;
(viii) SEQ ID NO: 114 and SEQ ID NO: 122;
(ix) SEQ ID NO: 130 and SEQ ID NO: 138;
(x) SEQ ID NO: 146 and SEQ ID NO: 154;
(xi) SEQ ID NO: 162 and SEQ ID NO: 170;
(xii) SEQ ID NO: 178 and SEQ ID NO: 186;

The present invention includes a polynucleotide comprising the following polynucleotide sets which encode a CDR-H1, CDR-H2, CDR-H3, CDR-L1, CDR-L2 and CDR-L3. The composition of the DNA sequence of those polynucleotides is set forth below in Table 4.

TABLE 4
DNA Sequences Encoding Heavy and Light CDRs in
Each Immunoglobulins of the Present Invention

| | CDR- | CDR- | CDR- | CDR- | CDR- | CDR- |
|---|---|---|---|---|---|---|
| Name | H1 | H2 | H3 | L1 | L2 | L3 |
| SM-05A | 3 | 5 | 7 | 11 | 13 | 15 |
| SM-05B | 19 | 21 | 23 | 27 | 29 | 31 |
| SM-05C | 35 | 37 | 39 | 43 | 45 | 47 |
| SM-05D | 51 | 53 | 55 | 59 | 61 | 63 |
| SM-05E | 67 | 69 | 71 | 75 | 77 | 79 |
| SM-05F | 83 | 85 | 87 | 91 | 93 | 95 |
| SM-05G | 99 | 101 | 103 | 107 | 109 | 111 |
| SM-05H | 115 | 117 | 119 | 123 | 125 | 127 |
| SM-05I | 131 | 133 | 135 | 139 | 141 | 143 |
| SM-05J | 147 | 149 | 151 | 155 | 157 | 159 |
| SM-05K | 163 | 165 | 167 | 171 | 173 | 175 |
| SM-05L | 179 | 181 | 183 | 187 | 189 | 191 |

*Numbers correspond to an amino acid sequence set forth in that SEQ ID NO

The present invention includes monoclonal anti-γc antigen-binding proteins, e.g., antibodies and antigen-binding fragments thereof, as well as monoclonal compositions comprising a plurality of isolated monoclonal antigen-binding proteins. The term “monoclonal antibody” or “mAb”, as used herein, refers to a member of a population of substantially homogeneous antibodies raised from single clone, i.e., the antibody molecules comprising the population are identical in amino acid sequence except for possible naturally occurring mutations that may be present in minor amounts. A “plurality” of such monoclonal antibodies and fragments in a composition refers to a concentration of identical (i.e., as discussed above, in amino acid sequence except for possible naturally occurring mutations that may be present in minor amounts) antibodies and fragments which is above that which would normally occur in nature, e.g., in the blood of a host organism such as a mouse or a human.

In some embodiments, the anti-γc antigen-binding protein provided herein isa chimeric antibody (e.g., antibody containing mouse variable regions (V_{H} and V_{L}) and human constant regions (C_{H} and C_{L}). In some embodiments, the anti-γc antigen-binding protein provided herein is a humanized antibody (e.g., antibody containing mouse CDRs (CDR-H1, CDR-H2, CDR-H3, CDR-L1, CDR-L2 and CDR-L3), human FRs (FR-H1, FR-H2, FR-H3, FR-H4, FR-L1, FR-L2, FR-L3, FR-L4) and human constant regions (C_{H} and C_{L})). In some embodiments, the amino acids in humanized FRs are back-mutated to the ones from original mouse FRs. In some embodiments, amino acids within CDRs are randomly mutated (e.g., arginine to glutamic acid) to increase the binding affinity. As used herein, a “chimeric antibody” is an antibody having the variable domain from a first antibody and the constant domain from a second antibody, where the first and second antibodies are from different species (see e.g., U.S. Pat. No. 4,816,567). As used herein, a “humanized antibody” in an antibody with segments of foreign-derived amino acids (e.g., CDRs from mouse origin) interspersed among variable domain segments of human-derived amino acid residues, and the humanized variable heavy and variable light domains are linked to heavy and light constant regions of human origin [Meyler's Side Effects of Drugs (Sixteenth Edition), 2016] set forth in SEQ ID NO: 201-202 or SEQ ID NO: 203-204. Methods for generating humanized antibodies are known in the art. The present invention uses the framework-patching method to generate humanized versions of anti-γc antigen-binding protein (see, e.g., U.S. Pat. No. 7,321,026B2). The resulting full-length humanized antibodies refer to hSM-05A and hSM-05E.

In some embodiments, the anti-γc antigen-binding protein provided herein is a mouse scFv fragment. As used herein, a “ScFv fragment” is a fusion protein of the variable regions of the V_{H} and V_{L} of immunoglobulins, connected with a short linker peptide of 10 to about 25 amino acids (e.g., G4S linker (SEQ ID NO: 236)). The ScFv offers a convenient way for pre-screening potential candidates with high binding affinity to γc protein and blocking activities to STAT phosphorylation.

The terms “epitope” (also called “antigenic determinant”) is used interchangeably herein and refer to the site on the surface of a target molecule to which an antibody or antigen-binding fragment binds, such as a localized region on the surface of an antigen. The target molecule can comprise, a protein, a peptide, a nucleic acid, a carbohydrate, or a lipid. An epitope having immunogenic activity is a portion of a target molecule recognized by the immune system (e.g., antibodies, B cells, or T cells) to elicit an immune response in an animal. An epitope of a target molecule having antigenic activity is a portion of the target molecule to which an antibody binds, as determined by any method well known in the art (e.g., an immunoassay). Antigenic epitopes need not necessarily be immunogenic. Epitopes often consist of chemically active surface groupings of molecules such as amino acids or sugar side chains and have specific three-dimensional structural characteristics as well as specific charge characteristics. The term, “epitope” includes linear epitopes and conformational epitopes. A region of a target molecule (e.g., a polypeptide) contributing to an epitope can be contiguous amino acids of the polypeptide or the epitope can come together from two or more non-contiguous regions of the target molecule. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, 6, 7, or 8-10 amino acids in a unique spatial conformation.

Methods for determining the epitope of an antigen-binding protein, e.g., antibody or fragment or polypeptide, include alanine scanning mutational analysis, peptide blot analysis [Reineke, Methods Mol. Biol., 2004; 248: 443-63], peptide cleavage analysis, crystallographic studies and NMR analysis. In addition, methods such as epitope excision, epitope extraction and chemical modification of antigens can be employed [Tomer, Prot. Sci., 2000; 9: 487-496)]. Other methods include hydrogen/deuterium exchange detected by mass spectrometry [Ehring, Analytical Biochemistry, 1999; 267: 52-25; Engen and Smith, Anal. Chem., 2001; 73: 256A-265A], shotgun mutagenesis [Davidson and Doranz, Immunology., 2014; 143 (1): 13-20] and conformational epitopes based on mass spectrometry and ultra violet photodissociation [Mehaffey et al., Anal Chem., 2020; 92 (17): 11869-11878.].

The term “specifically binds,” as used herein, refers to a situation that a polypeptide or molecule interacts more frequently, more rapidly, with greater duration, with greater affinity, or with some combination of the above to the epitope, protein, or target molecule than with alternative substances, including related and unrelated proteins. A binding moiety (e.g., antibody) that specifically binds a target molecule (e.g., antigen) can be identified by e.g., immunoassays, ELISAs, Bio-Layer Interferometry (“BLI”), SPR (e.g., Biacore), or other techniques known to those of skills in the art. Typically, a specific reaction will be at least twice background signal or noise and can be 10 times higher than background. See, e.g., Paul, ed., 1989, FUNDAMENTAL IMMUNOLOGY SECOND EDITION, Raven Press, New York at pages 332-336 for a discussion regarding antibody specificity. In some embodiments, a binding moiety that specifically binds a target molecule can bind the target molecule with an affinity that is at least 20, 30, 40, 50, 60, 70, 80, 90 or 100 times greater, than its affinity for a different molecule. In some embodiments, a binding moiety that specifically binds a particular target molecule binds a different molecule at such a low affinity that binding cannot be detected using an assay described herein or otherwise known in the art. In some embodiments, “specifically binds” means, for instance, that a binding moiety binds a molecule target with a K_{D} of about 0.1 mM or less. In some embodiments, “specifically binds” means that a polypeptide or molecule binds a target with a K_{D} of at about 10 μM or less or about 1 μM or less. In some embodiments, “specifically binds” means that a polypeptide or molecule binds a target with a K_{D} of at about 0.1 μM or less, about 0.01 μM or less, or about 1 nM or less. Because of the sequence identity between homologous proteins in different species, specific binding can include a polypeptide or molecule that recognizes a protein or target in more than one species. Likewise, because of homology within certain regions of polypeptide sequences of different proteins, specific binding can include a polypeptide or molecule that recognizes more than one protein or target. It is understood that, in some embodiments, a binding moiety (e.g., antibody) that specifically binds a first target may or may not specifically bind a second target. As such, “specific binding” does not necessarily require (although it can include) exclusive binding, i.e., binding to a single target. Thus, a binding moiety (e.g., antibody) can, in some embodiments, specifically bind more than one tar get.

The term “binding affinity” as used herein generally refers to the strength of the sum total of noncovalent interactions between a binding moiety (e.g., antibody) and a target molecule (e.g., antigen). The binding between a binding moiety and a target molecule is a reversible process, and the affinity of the binding is typically reported as an equilibrium dissociation constant (K_{D}). K_{D} is the ratio of a dissociation rate (k_{off} or k_{d}) to the association rate (kₒₙ or kₐ). The lower the K_{D} of a binding pair, the higher the affinity. K_{A} is the equilibrium association constant, which is also the reciprocal of the equilibrium dissociation constant, i.e., =1/K_{D}. For an antibody-antigen interaction, K_{D} can be calculated as the ratio of the products of concentrations of free antibody and free antigen over the concentrations of antibody-antigen complex, i.e., [antigen]×[antibody]/[antigen-antibody].

A variety of methods of measuring binding affinity are known in the art, any of which can be used for purposes of the present disclosure. Specific illustrative embodiments include the following. In some embodiments, the “K_{D}” or “K_{D} value” can be measured by assays known in the art, for example by a binding assay. The K_{D} may be measured in a radiolabeled antigen binding assay (RIA) (Chen, Y et al. Journal of molecular biology vol. 293, 4 (1999): 865-81). The K_{D} or K_{D} value can also be measured by using biolayer interferometry (BLI) using, for example the Gator system (Probe Life), or the Octet-96 system (Sartorius, Gottingen, Germany). The K_{D} or K_{D} value can also be measured by using surface plasmon resonance assays by using a BIAcore system (e.g., Pharmacia Biosensor AB, Uppsala, Sweden and Piscataway, NJ).

The term “variant” as used herein in relation to a protein or a polypeptide with particular sequence features (the “reference protein” or “reference polypeptide”) refers to a different protein or polypeptide having one or more amino acid substitutions, deletions, and/or additions as compared to the reference protein or reference polypeptide. The changes to an amino acid sequence can be amino acid substitutions. The changes to an amino acid sequence can be conservative amino acid substitutions. A functional fragment or a functional variant of a protein or polypeptide maintains the basic structural and functional properties of the reference protein or polypeptide.

The terms “polypeptide,” “peptide,” “protein,” and their grammatical equivalents as used interchangeably herein refer to polymers of amino acids of any length, which can be linear or branched. It can include unnatural, modified amino acids or be interrupted by non-amino acids. A polypeptide, peptide, or protein can also be modified with, e.g., disulfide bond formation, glycosylation, lipidation, acetylation, phosphorylation, or any other manipulation or modification.

The terms “polynucleotide”, “nucleic acid” and their grammatical equivalents as used interchangeably herein mean polymers of nucleotides of any length and include DNA and RNA. The nucleotides can be deoxyribonucleotides, ribonucleotides, modified nucleotides or bases, and/or their analogs, or any substrate that can be incorporated into a polymer by DNA or RNA polymerase. A nucleic acid molecule can be single-stranded or double-stranded.

As used herein, the term “encode” and its grammatical equivalents re fer to the inherent property of specific sequences of nucleotides in a polynucleotide or a nucleic acid, such as a gene, a cDNA, or an mRNA, to serve as templates for syn thesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein. Unless otherwise specified, a “nucleotide sequence encoding an amino acid sequence” includes all nucleotide sequences that are degenerate versions of each other and that encode the same amino acid sequence. Nucleotide sequences that encode proteins and RNA can include introns.

A polypeptide, peptide, protein, antibody, polynucleotide, vector, cell, or composition which is “isolated” is a polypeptide, peptide, protein, antibody, polynucleotide, vector, cell, or composition which is in a form not found in nature. Isolated polypeptides, peptides, proteins, antibodies, polynucleotides, vectors, cells, or compositions include those which have been purified to a degree that they are no longer in a form in which they are found in nature. In some embodiments, a polypeptide, peptide, protein, antibody, polynucleotide, vector, cell, or composition which is isolated is substantially pure. In some embodiments, a polypeptide, peptide, protein, antibody, polynucleotide, vector, cell, or composition which is isolated is substantially free of other cellular material and/or chemicals.

The terms “identical”, “identity”, “percentage identical” and their grammatical equivalents as used herein in the context of two or more polynucleotides or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software that can be used to obtain alignments of amino acid or nucleotide sequences are well-known in the art. These include, but are not limited to, BLAST, ALIGN, Megalign, BestFit, GCG Wisconsin Package, and variants thereof. In some embodiments, two polynucleotides or polypeptides provided herein are substantially identical, meaning they have at least 70%, at least 75%, at least 80%, at least 85%, at least 90%, and in some embodiments at least 95%, at least 96%, at least 97%, at least 98%, or at least 99% nucleotide or amino acid residue identity, when compared and aligned for maximum correspondence, as measured using a sequence comparison algorithm or by visual inspection. In some embodiments, identity exists over a region of the amino acid sequences that is at least about 10 residues, at least about 20 residues, at least about 40-60 residues, at least about 60-80 residues in length or any integral value there between. In some embodiments, identity exists over a longer region than 60-80 residues, such as at least about 80-100 residues, and in some embodiments the sequences are substantially identical over the full-length of the sequences being compared, such as the coding region of a target protein or an antibody. In some embodiments, identity exists over a region of the nucleotide sequences that is at least about 10 bases, at least about 20 bases, at least about 40-60 bases, at least about 60-80 bases in length or any integral value there between. In some embodiments, identity exists over a longer region than 60-80 bases, such as at least about 80-1000 bases or more, and in some embodiments the sequences are substantially identical over the full-length of the sequences being compared, such as a nucleotide sequence encoding a protein of interest.

A “conservative amino acid substitution” as used herein, is one in which one amino acid residue is replaced with another amino acid residue having a simi lar side chain. Amino acid or residue that is “conservatively similar” as used herein refers to non-identical amino acid residue having similar side chains. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aroma tic side chains (e.g., tyrosine, phenylalanine, tryptophan, histidine).

The present invention includes antigen-binding proteins that compete for binding to γc, e.g., a variant γc epitope as discussed herein, with an antigen-binding protein of the present invention, e.g., SM-05A, SM-05B, SM-05C, SM-05D, SM-05E, SM-05F, SM-05G, SM-05H, SM-05I, SM05J, SM-05K, SM-05L. The term “competes” and their grammatical equivalents as used herein, refers to an antigen-binding protein (e.g., antibody or antigen-binding fragment thereof) that binds to an antigen (e.g., γc) and inhibits or blocks the binding of another antigen-binding protein (e.g., antibody or antigen binding fragment thereof) to the antigen. Unless otherwise stated, the term also includes competition between two antigen-binding proteins, e.g., antibodies, in both orientations, i.e., a first antibody that binds antigen and blocks binding by a second antibody and vice versa. Thus, in an embodiment of the invention, competition occurs in one such orientation. In certain embodiments, the first and second antigen-binding proteins (e.g., antibodies) may bind to the same epitope. Alternatively, the first and second antigen-binding proteins (e.g., antibodies) may bind to different, but, for example, overlapping or non-overlapping epitopes, wherein binding of one inhibits or blocks the binding of the second antibody, e.g., via steric hindrance or conformational change. Competition between antigen binding proteins (e.g., antibodies) may be determined by methods known in the art, for example, by a competitive standard enzyme-linked immunosorbent assay (ELISA) assay. Also, the competition may be measured by a real-time, label-free bio-layer interferometry assay using Octet RED384 biosensor.

Typically, an antibody or antigen-binding fragment of the invention which is modified in some ways retains the specific binding affinity to γc, e.g., retains at least 10% of its γc binding activity (when compared to the parental antibody) when that activity is expressed on a molar basis. Preferably, an antibody or antigen-binding fragment of the invention retains at least 20%, 50%, 70%, 80%, 90%, 95% or 100% or more of the γc binding affinity as the parental antibody. It is also intended that an antibody or antigen binding fragment of the invention may include conservative or non-conservative amino acid substitutions (referred to as “conservative variants” or “function conserved variants” of the antibody) that do not substantially alter its normal biological function (e.g., blocking γc cytokine induced signaling pathway).

A “variant” of a polypeptide, such as an immunoglobulin chain (e.g., SM-05A, SM-05B, SM-05C, SM-05D, SM-05E, SM-05F, SM-05G, SM-05H, SM-05I, SM05J, SM-05K, SM-05L; V_{H}, V_{L}, HC or LC or CDR thereof comprising the amino acid sequence specifically set forth herein), refers to a polypeptide comprising an amino acid sequence that is at least 70-99.9% (e.g., at least 70, 72, 74, 75, 76, 79, 80, 81, 82, 83, 84, 85, 86, 87, 88, 89, 90, 91, 92, 93, 94, 95, 96, 97, 98, 99, 99.5 or 99.9%) identical or similar to a referenced amino acid sequence that is set forth herein (e.g., any of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190 and/or 192); when the comparison is performed by a BLAST algorithm.

Moreover, a variant of a polypeptide may include a polypeptide such as an immunoglobulin chain (e.g., SM-05A, SM-05B, SM-05C, SM-05D, SM-05E, SM-05F, SM-05G, SM-05H, SM-05I, SM05J, SM-05K, SM-05L; V_{H}, V_{L}, HC or LC or CDR thereof) which may include the amino acid sequence of the reference polypeptide whose amino acid sequence is specifically set forth herein but for one or more (e.g., 1, 2, 3, 4, 5, 6, 7, 8, 9 or 10) modulations, e.g., one or more missense mutations (e.g., conservative substitutions), non-sense mutations, deletions, or insertions. In an embodiment of the invention, an γc binding protein includes an immunoglobulin heavy chain variant comprising CDR-H1, CDR-H2 and CDR-H3 wherein one or more (e.g., 1 or 2 or 3) of such CDRs has one or more of such modulations (e.g., conservative substitutions) and/or an immunoglobulin light chain variant comprising CDR-L1, CDR-L2 and CDR-L3 wherein one or more (e.g., 1 or 2 or 3) of such CDRs has one or more of such mutations (e.g., conservative substitutions).

### Generation of Phage Display Library and Production of Human γc Binding Proteins

Phage display library is an advanced technology to the traditional hybridoma technology for generation and screening of antigen specific monoclonal antibodies. The fragment of protein of interest is “displayed” on the sur face of filamentous phages (e.g., M13 bacteriophage) after inserting a foreign polynucleotide (e.g., anti-γc ScFv sequence) into the vector encoding the filamentous phage coat protein gene (e.g., pCANTAB5). The resulting engineered phages could be utilized for selecting the γc binding protein fragments with high affinity to the γc protein.

The term “vector,” and its grammatical equivalents as used herein refer to a vehicle that is used to carry genetic material (e.g., a polynucleotide sequence), which can be introduced into a host cell, where it can be replicated and/or expressed. Vectors applicable for use include, for example, expression vectors, plasmids, phagemids, viral vectors, episomes and artificial chromosomes, which can include selection sequences or markers operable for stable integration into a host cell's chromosome. Addition ally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more polynucleotides are to be co-expressed, both polynucleotides can be inserted, for example, into a single expression vector or in separate expression vectors. For single vector expression, the encoding polynucleotides can be operationally linked to one common expression control sequence or linked to different expression control sequences, such as one inducible promoter and one constitutive promoter. The introduction of polynucleotides into a host cell can be confirmed using methods well known in the art. It is understood by those skills in the art that the polynucleotides are expressed in a sufficient amount to produce a desired product (e.g., an IgG consisting γc bin ding proteins), and it is further understood that expression levels can be optimized to obtain sufficient expression using methods well known in the art.

As used herein, the term “host cell” refers to a cell into which a genetical material, such as a recombinant expression vector can be introduced or has been introduced. Host cells include not only the subject cell introduced with the exogenous genetic material, but also the progeny of such a cell. Because certain modifications may occur in succeeding generations due to either mutation or environmental influences, such progeny may not be identical to the parent cell.

Eukaryotic and prokaryotic host cells, including mammalian cells, may be used as hosts for expression of an γc binding protein (e.g., antibody or antigen-binding fragment thereof). Such host cells are well known in the art and many are available from the American Type Culture Collection (ATCC). These host cells include, inter alia, Chinese hamster ovary (CHO) cells, murine myeloma cells (NS0), SP2 cells, HeLa cells, baby hamster kidney (BHK) cells, monkey kidney cells (COS), human hepatocellular carcinoma cells (e.g., Hep G2), A549 cells, 3T3 cells, H EK-293 cells and a number of other cell lines from different mammalian species. Other cell lines that may be used are insect cell lines (e.g., Spodoptera frugiperda or Trichoplusiani), amphibian cells, bacterial cells (e.g., TOP10 Escherichia coli, TG1 Escherichia coli, HB2151 Escherichia coli), plant cells and fungal cells. Fungal cells include yeast and filamentous fungus cells including, e.g., Pichia, Pichia pastoris, Pichia fin landica, Pichia trehalophila, Pichia koclamae, Pichia membranaefaciens, Pichia minuta (Ogataea minuta, Pichia lindnen), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens and Neurospora crassa. The present invention includes an isolated host cell (e.g., a Expi-CHO cell or any type of host cell set forth above) comprising an antigen-binding protein, a V_{H}, V_{L}, HC, LC or CDRs thereof (or variant thereof), such as SM-05A, SM-05B, SM-05C, SM-05D, SM-05E, SM-05F, SM-05G, SM-05H, SM-05I, SM05J, SM-05K, SM-05L; and/or a polynucleotide encoding one or more immunoglobulin cha ins thereof (e.g., as discussed above).

Human γc protein (extracellular domain) with murine Fc tag set forth in SEQ ID NO: 201 was generated using the host cell system (e.g., Expi-CHO cell) and purified using the ProSep Ultra Plus. To remove the contaminants and maintain the physiological pH value, purified γc protein was buffered exchange into PBS solution using the Amicon ultra-15 10k centrifugal filter. The resulting protein was diluted to 1 mg/ml in PBS for storage.

Purified human γc protein was immunized to the mice as shown in Table 5. ELISA titration of test-bleeds was performed between the immunization steps to evaluate the successful generation of antibodies in the animals.

TABLE 5
Immunization procedure of human γc protein

| Day | Procedure |
|---|---|
| 0 | Pre-immune serum collection. |
| | Immunization of each mouse with 50 |
| | μg antigen in Complete Freund Adjuvant (CFA). |
| 14 | Immunization of each mouse with |
| | 50 μg antigen in Incomplete |
| | Freund Adjuvant (IFA). |
| 28 | Immunization of each mouse with |
| | 50 μg antigen in IFA. |
| 35 | ELISA titration of test-bleeds. |
| 42 | Immunization of each mouse with |
| | 50 μg antigen in IFA. |
| 49 | ELISA titration of test-bleeds. |
| 56 | Immunization of each mouse with |
| | 50 μg antigen in IFA. |
| 63 | ELISA titration of test-bleeds. |

Total RNA was isolated from mouse spleen at the end of immunization. All V_{H} and V_{L} fragments were identified and clones using the degenerate primers. Random combination of all V_{H} and V_{L} fragments with a linker region (e.g., G4S linker (SEQ ID NO: 236)) were performed to generate ScFv library. To prepare a scFv gene, the V_{H}- and V_{L}-encoding DNA fragments are operatively linked to another fragment encoding a flexible linker, e.g., encoding the amino acid sequence (Gly4-Ser) 3 (SEQ ID NO: 237), such that the V_{H} and V_{L} sequences can be expressed as a contiguous single-chain protein, with the V_{L} and V_{H} regions joined by the flexible linker [Bird, R E et al. Science (New York, N.Y.) vol. 242, 4877 (1988): 423-6; Huston, J S et al. Proceedings of the National Academy of Sciences of the United States of America vol. 85, 16 (1988): 5879-83; McCafferty, J et al. Nature vol. 348, 6301 (1990): 552-4].

After ligation of ScFv library to the phage vector (e.g., pCANTAB5), the vector was transfected into the bacterial host cell (e.g., TOP10 competent E. coli) for expansion of phage library harboring all combination of ScFv fragments. To select the phage showing strong binding affinity to human γc protein, phage library was screened against decreasing concentrations of human γc protein in the 96-well plate. After two to three rounds of selection, individual phage was grown in a single clone format to evaluate the binding affinity of each clone through monoclonal phage ELISA as shown in Table 6. Clones with 5-fold higher OD value than background (i.e.: 0.3) were selected for DNA sequencing. The finalized DNA and amino acid sequences are set forth in SEQ ID NO: 1-192.

TABLE 6
Result for monoclonal phage ELISA to human γc protein

| Clones | OD (450 nm) |
|---|---|
| 1 | 0.095 |
| 2 | 1.849 |
| 3 | 0.066 |
| 4 | 1.87 |
| 5 | 0.065 |
| 6 | 0.074 |
| 7 | 0.226 |
| 8 | 0.057 |
| 9 | 2.978 |
| 10 | 0.051 |
| 11 | 2.85 |
| 12 | 0.052 |
| 13 | 0.061 |
| 14 | 0.067 |
| 15 | 1.29 |
| 16 | 0.065 |
| 17 | 1.769 |
| 18 | 0.051 |
| 19 | 0.051 |
| 20 | 2.643 |
| 21 | 2.234 |
| 22 | 0.067 |
| 23 | 0.108 |
| 24 | 1.639 |
| 25 | 0.059 |
| 26 | 2.484 |
| 27 | 0.054 |
| 28 | 0.067 |
| 29 | 0.055 |
| 30 | 0.055 |
| 31 | 0.046 |
| 32 | 1.831 |
| 33 | 0.055 |
| 34 | 0.058 |
| 35 | 0.051 |
| 36 | 0.068 |
| 37 | 0.059 |
| 38 | 2.342 |
| 39 | 0.11 |
| 40 | 0.051 |
| 41 | 0.057 |
| 42 | 0.057 |
| 43 | 2.775 |
| 44 | 0.053 |
| 45 | 0.052 |
| 46 | 0.059 |
| 47 | 0.094 |
| 48 | 0.071 |
| 49 | 1.784 |
| 50 | 2.68 |
| 51 | 0.069 |
| 52 | 1.470 |
| 53 | 0.078 |
| 54 | 0.082 |
| 55 | 1.593 |
| 56 | 0.068 |
| 57 | 2.936 |
| 58 | 0.046 |
| 59 | 0.053 |
| 60 | 0.075 |
| 61 | 0.089 |
| 62 | 0.915 |
| 63 | 0.098 |
| 64 | 1.734 |
| 65 | 1.76 |
| 66 | 0.049 |
| 67 | 1.898 |
| 68 | 0.063 |
| 69 | 2.037 |
| 70 | 1.699 |
| 71 | 0.087 |
| 72 | 0.064 |
| 73 | 0.051 |
| 74 | 0.047 |
| 75 | 0.048 |
| 76 | 0.063 |
| 77 | 0.068 |
| 78 | 0.097 |
| 79 | 0.086 |
| 80 | 0.062 |
| 81 | 0.053 |
| 82 | 1.882 |
| 83 | 0.047 |
| 84 | 2.818 |
| 85 | 0.072 |
| 86 | 0.095 |
| 87 | 0.080 |
| 88 | 0.064 |
| 89 | 1.706 |
| 90 | 1.627 |
| 91 | 0.069 |
| 92 | 0.088 |
| 93 | 0.057 |
| 94 | 0.051 |
| 95 | 0.053 |
| 96 | 0.057 |
| 97 | 0.053 |
| 98 | 2.353 |
| 99 | 0.050 |
| 100 | 0.046 |
| 101 | 0.046 |
| 102 | 1.767 |
| 103 | 0.557 |
| 104 | 0.050 |
| 105 | 1.784 |
| 106 | 0.087 |
| 107 | 1.995 |
| 108 | 0.051 |
| 109 | 0.047 |
| 110 | 1.686 |
| 111 | 0.045 |
| 112 | 1.837 |
| 113 | 0.048 |
| 114 | 0.052 |
| 115 | 0.064 |
| 116 | 2.043 |
| 117 | 0.045 |
| 118 | 0.046 |
| 119 | 0.870 |
| 120 | 0.047 |
| 121 | 0.047 |
| 122 | 0.049 |
| 123 | 0.062 |
| 124 | 0.049 |
| 125 | 0.048 |
| 126 | 0.049 |
| 127 | 2.560 |
| 128 | 0.055 |
| 129 | 1.866 |
| 130 | 0.054 |
| 131 | 2.057 |
| 132 | 0.055 |
| 133 | 0.047 |
| 134 | 0.462 |
| 135 | 1.812 |
| 136 | 0.049 |
| 137 | 0.048 |
| 138 | 0.057 |
| 139 | 0.056 |
| 140 | 0.053 |
| 141 | 1.649 |
| 142 | 2.753 |
| 143 | 0.055 |
| 144 | 0.456 |
| 145 | 0.056 |
| 146 | 1.319 |
| 147 | 0.049 |
| 148 | 0.049 |
| 149 | 0.049 |
| 150 | 0.050 |
| 151 | 2.868 |
| 152 | 1.727 |
| 153 | 0.053 |
| 154 | 1.723 |
| 155 | 0.055 |
| 156 | 0.047 |
| 157 | 0.050 |
| 158 | 0.048 |
| 159 | 0.048 |
| 160 | 0.050 |
| 161 | 0.050 |
| 162 | 0.061 |
| 163 | 0.145 |
| 164 | 0.049 |
| 165 | 1.494 |
| 166 | 1.659 |
| 167 | 0.049 |
| 168 | 0.048 |
| 169 | 0.057 |
| 170 | 0.058 |
| 171 | 0.051 |
| 172 | 0.047 |
| 173 | 0.050 |
| 174 | 0.052 |
| 175 | 0.049 |
| 176 | 0.050 |
| 177 | 1.671 |
| 178 | 0.056 |
| 179 | 0.049 |
| 180 | 0.259 |
| 181 | 0.044 |
| 182 | 0.052 |
| 183 | 0.453 |
| 184 | 1.480 |
| 185 | 0.078 |
| 186 | 0.060 |
| 187 | 0.056 |
| 188 | 0.074 |
| 189 | 0.062 |
| 190 | 0.069 |
| 191 | 0.063 |
| 192 | 2.723 |
| 193 | 1.896 |
| 194 | 0.055 |
| 195 | 0.051 |
| 196 | 0.056 |
| 197 | 0.213 |
| 198 | 0.069 |
| 199 | 0.622 |
| 200 | 2.044 |
| 201 | 0.050 |
| 202 | 1.796 |
| 203 | 0.436 |
| 204 | 1.693 |
| 205 | 0.054 |
| 206 | 0.059 |
| 207 | 0.645 |
| 208 | 0.051 |
| 209 | 1.969 |
| 210 | 2.046 |
| 211 | 0.050 |
| 212 | 0.188 |
| 213 | 0.056 |
| 214 | 0.058 |
| 215 | 0.056 |
| 216 | 0.049 |
| 217 | 1.789 |
| 218 | 1.039 |
| 219 | 1.715 |
| 220 | 0.053 |
| 221 | 0.063 |
| 222 | 0.057 |
| 223 | 2.387 |
| 224 | 0.051 |
| 225 | 0.053 |
| 226 | 0.053 |
| 227 | 0.052 |
| 228 | 0.770 |
| 229 | 0.059 |
| 230 | 2.815 |
| 231 | 1.354 |
| 232 | 0.624 |
| 233 | 2.481 |
| 234 | 0.054 |
| 235 | 0.054 |
| 236 | 0.060 |
| 237 | 0.059 |
| 238 | 0.060 |
| 239 | 2.893 |
| 240 | 0.052 |
| 241 | 2.787 |
| 242 | 0.054 |
| 243 | 1.682 |
| 244 | 0.460 |
| 245 | 1.725 |
| 246 | 0.060 |
| 247 | 1.870 |
| 248 | 0.048 |
| 249 | 0.049 |
| 250 | 0.612 |
| 251 | 2.025 |
| 252 | 0.052 |
| 253 | 0.052 |
| 254 | 0.053 |
| 255 | 0.052 |
| 256 | 0.047 |
| 257 | 1.597 |
| 258 | 0.051 |
| 259 | 0.050 |
| 260 | 2.960 |
| 261 | 0.057 |
| 262 | 0.563 |
| 263 | 0.051 |
| 264 | 2.089 |
| 265 | 0.052 |
| 266 | 1.204 |
| 267 | 2.752 |
| 268 | 0.054 |
| 269 | 0.058 |
| 270 | 0.305 |
| 271 | 0.055 |
| 272 | 0.047 |
| 273 | 0.052 |
| 274 | 0.393 |
| 275 | 0.051 |
| 276 | 0.055 |
| 277 | 0.072 |
| 278 | 1.885 |
| 279 | 0.056 |
| 280 | 0.057 |
| 281 | 0.058 |
| 282 | 0.371 |
| 283 | 1.728 |
| 284 | 0.047 |
| 285 | 0.047 |
| 286 | 0.049 |
| 287 | 0.049 |
| 288 | 0.051 |
| 289 | 0.054 |
| 290 | 0.151 |
| 291 | 0.047 |
| 292 | 0.047 |
| 293 | 1.592 |
| 294 | 0.051 |
| 295 | 0.786 |
| 296 | 0.052 |
| 297 | 0.054 |
| 298 | 0.057 |
| 299 | 0.046 |
| 300 | 0.694 |
| 301 | 0.047 |
| 302 | 2.784 |
| 303 | 0.047 |
| 304 | 1.743 |
| 305 | 0.051 |
| 306 | 0.621 |
| 307 | 0.058 |
| 308 | 0.050 |
| 309 | 0.479 |
| 310 | 1.839 |
| 311 | 0.047 |
| 312 | 2.809 |
| 313 | 0.055 |
| 314 | 0.057 |
| 315 | 0.051 |
| 316 | 0.051 |
| 317 | 0.053 |
| 318 | 0.050 |
| 319 | 0.048 |
| 320 | 0.052 |
| 321 | 0.058 |
| 322 | 0.057 |
| 323 | 0.046 |
| 324 | 0.045 |
| 325 | 0.043 |
| 326 | 0.047 |
| 327 | 0.044 |
| 328 | 0.049 |
| 329 | 0.048 |
| 330 | 0.055 |
| 331 | 0.049 |
| 332 | 0.047 |
| 333 | 0.045 |
| 334 | 0.047 |
| 335 | 0.045 |
| 336 | 0.050 |
| 337 | 0.050 |
| 338 | 0.051 |
| 339 | 0.049 |
| 340 | 0.044 |
| 341 | 0.045 |
| 342 | 0.046 |
| 343 | 0.046 |
| 344 | 0.046 |
| 345 | 0.048 |
| 346 | 0.052 |
| 347 | 0.048 |
| 348 | 0.048 |
| 349 | 2.349 |
| 350 | 0.047 |
| 351 | 0.045 |
| 352 | 0.048 |
| 353 | 0.047 |
| 354 | 0.054 |
| 355 | 0.049 |
| 356 | 0.048 |
| 357 | 0.047 |
| 358 | 0.048 |
| 359 | 0.049 |
| 360 | 0.052 |
| 361 | 0.051 |
| 362 | 0.050 |
| 363 | 0.053 |
| 364 | 0.054 |
| 365 | 0.054 |
| 366 | 0.065 |
| 367 | 0.045 |
| 368 | 0.044 |
| 369 | 0.046 |
| 370 | 0.049 |
| 371 | 0.046 |
| 372 | 0.044 |
| 373 | 0.047 |
| 374 | 0.053 |
| 375 | 0.044 |
| 376 | 0.043 |
| 377 | 0.045 |
| 378 | 0.045 |
| 379 | 0.044 |
| 380 | 0.044 |
| 381 | 0.051 |
| 382 | 0.051 |
| 383 | 0.045 |
| 384 | 0.042 |
| 385 | 0.042 |
| 386 | 0.042 |
| 387 | 0.046 |
| 388 | 0.046 |
| 389 | 0.045 |
| 390 | 0.051 |
| 391 | 0.045 |
| 392 | 0.044 |
| 393 | 0.045 |
| 394 | 0.045 |
| 395 | 0.046 |
| 396 | 0.045 |
| 397 | 0.046 |
| 398 | 0.047 |
| 399 | 0.059 |
| 400 | 0.042 |
| 401 | 0.046 |
| 402 | 0.045 |
| 403 | 0.044 |
| 404 | 0.043 |
| 405 | 0.046 |
| 406 | 0.049 |
| M13KO7 (negative) | 0.045 |
| 1% Milk in PBS | 0.051 |

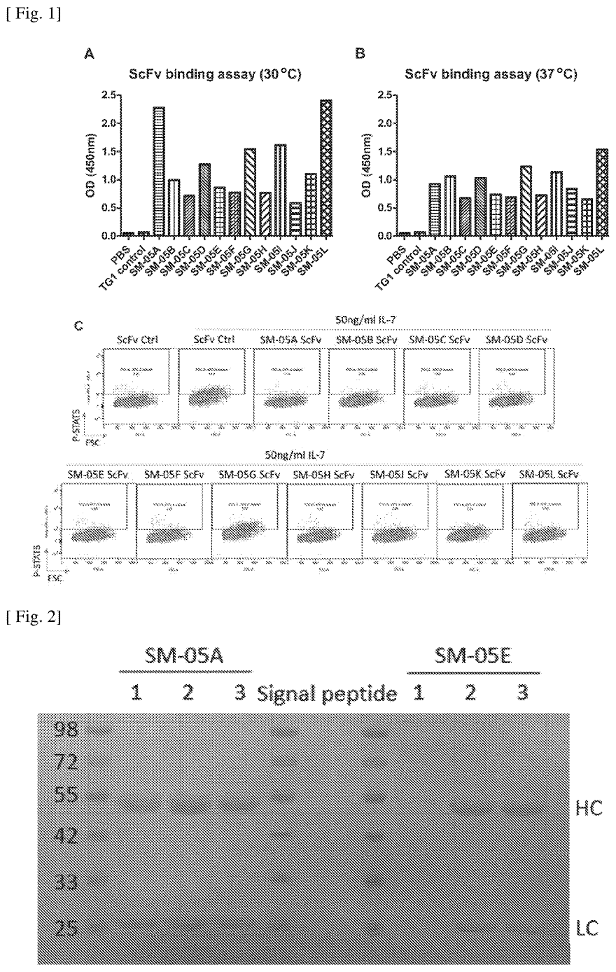

Soluble ScFv was generated using the E. coli strain HB2151 after transfection of phagemid into respective host. The periplasmic extract of bacteria containing soluble ScFv was harvested using ultra-sonication and purified using Capto™ L. After measuring the concentrations of crude ScFv extracts, the binding affinities of ScFv to human γc protein were evaluated through ELISA using anti bodies targeting E-Tag on ScFv. Results were summarized in FIG. 1A-B.

To screen the potencies of anti-γc ScFv, several cell lines were pre-incubated with different anti-γc ScFv (20 μg/ml) for an hour before the stimulation of individual cytokine (50 ng/ml) for 15 minutes. Cells were harvested and stained with FITC conjugated anti-pSTAT6, PE conjugated anti-pSTAT3 or APC con jugated anti-pSTAT5 antibodies for flow cytometry analyses. In some embodiments, the purified ScFv could block the IL-4 induced STAT6 phosphorylation in cell culture (e.g., Ramos). In some embodiments, the purified ScFv could block the IL-7 induced STAT5 phosphorylation in cell culture (e.g., HPB-ALL). In some embodiments, the purified ScFv could block the IL-9 induced STAT3 phosphorylation in cell culture (e.g., Jurkat). In some embodiments, the purified ScFv could block the IL-15 induced STAT5 phosphorylation in cell culture (e.g., KHYG-1). In some embodiments, the purified ScFv could block the IL-21 induced STAT3 phosphorylation in cell culture (e.g., Ramos). Results were summarized in Tables 7-10. Representative flow cytometry graphs for p-STAT5 blockades by anti-γc ScFvs in HPB-ALL were shown in FIG. 1C.

TABLE 7
Inhibition of IL-15 induced STAT phosphorylation

| ScFv | p-STAT5 inhibition (%) |
|---|---|
| SM-05A | 48.59 |
| SM-05B | 47.06 |
| SM-05C | 46.04 |
| SM-05D | 48.59 |
| SM-05E | 47.83 |
| SM-05F | 43.73 |
| SM-05H | 51.66 |
| SM-05I | 38.87 |
| SM-05J | 42.71 |
| SM-05K | 42.97 |
| SM-05L | 49.10 |

TABLE 8a
Inhibition of IL-4 induced STAT phosphorylation

| ScFv | p-STAT6 inhibition (%) |
|---|---|
| SM-05A | 86.10 |
| SM-05B | 76.95 |
| SM-05C | 45.99 |
| SM-05D | 63.77 |
| SM-05E | 85.63 |
| SM-05F | 45.99 |
| SM-05H | 63.36 |
| SM-05I | 74.06 |
| SM-05J | 82.22 |
| SM-05L | 73.54 |

TABLE 8b
Inhibition of IL-21 induced STAT phosphorylation

| ScFv | p-STAT3 inhibition (%) |
|---|---|
| SM-05A | 47.31 |
| SM-05B | 13.85 |
| SM-05C | 29.10 |
| SM-05D | 30.00 |
| SM-05E | 60.64 |
| SM-05F | 78.97 |
| SM-05H | 70.64 |
| SM-05I | 91.03 |
| SM-05J | 75.77 |
| SM-05K | 90.77 |
| SM-05L | 26.03 |

TABLE 9
Inhibition of IL-7 induced STAT phosphorylation

| ScFv | p-STAT5 inhibition (%) |
|---|---|
| SM-05A | 100 |
| SM-05B | 94.50 |
| SM-05C | 100 |
| SM-05D | 99.80 |
| SM-05E | 98.30 |
| SM-05F | 99.42 |
| SM-05G | 0 |
| SM-05H | 100 |
| SM-05J | 98.10 |
| SM-05K | 86.02 |
| SM-05L | 100 |

TABLE 10
Inhibition of IL-9 induced STAT phosphorylation

| ScFv | p-STAT3 inhibition (%) |
|---|---|
| SM-05A | 80.56 |
| SM-05B | 82.64 |
| SM-05C | 97.92 |
| SM-05D | 100 |
| SM-05E | 74.31 |
| SM-05F | 92.36 |
| SM-05H | 93.06 |
| SM-05I | 100 |
| SM-05J | 100 |
| SM-05L | 92.36 |

Methods of antibody production are well-known in the art. See for example, in Harlow et al., ANTIBODIES: A LABORATORY MANUAL, (Cold Spring Harbor Laboratory Press, 2nd ed. 1988); Hammerling et al., in: Monoclonal Antibodies and T-Cell Hybridomas 563, 681 (Elsevier, N.Y., 1981), each of which is incorporated herein in its entirety by reference.

In some embodiments, the γc binding protein that can be used in methods provided herein are recombinant, namely, prepared, expressed, produced or isolated by recombinant means. In some embodiments, the γc binding protein disclosed herein can be prepared, for example, by introducing recombinant expression vectors into host cells, a recombinant, combinatorial human antibody library, antibodies isolated from an animal (e.g., a mouse) that is transgenic for human immunoglobulin genes [Taylor, L D et al. Nucleic acids research vol. 20, 23 (1992): 6287-95] or antibodies prepared, expressed, produced, or isolated by any other means that involves splicing of human immunoglobulin gene sequences to other DNA sequences.

In some embodiments, the γc binding protein can be prepared by recombinant expression of immunoglobulin light and heavy chain genes in a host cell (e.g., Expi-CHO cells). To express an γc binding protein, a host cell is introduced with one or more recombinant expression vectors carrying DNA fragments encoding the immunoglobulin light and heavy chains of the γc binding protein such that the light and heavy chains are expressed in the host cell and, preferably, secreted into the medium in which the host cells are cultured, from which medium the γc binding protein can be recovered. Standard recombinant DNA methodologies are used to obtain the γc binding protein heavy and light chain genes, incorporate these genes into recombinant expression vectors and introduce the vectors into host cells, such as those described in Sambrook, Fritsch and Maniais (eds), MOLECULAR CLONING: A LABORATORY MANUAL, Second Edition, Cold Spring Harbor, N.Y., (1989), Ausubel et al. (eds.) CURRENT PROTOCOLS IN MOLECULAR BIOLOGY, Greene Publishing Associates, (1989) and in U.S. Pat. No. 4,816,397.

To express a recombinant γc binding protein, DNA fragments encoding the light and heavy chain variable regions are first obtained. These DNAs can be obtained by amplification and modification of hybridomas for the murine antibody light and heavy chain variable sequences using the polymerase chain reaction (PCR), or by oligosynthesis based on the encoded amino acid sequence of design light and heavy chain variable sequences using standard methods known to those skilled in the art. The encoding DNA sequences can be further optimized to facilitate mammalian expression of the resultant antibody.

The VH and VL fragments for the murine antibody can be further mutated to encode the humanized antibodies using framework-patching method (see, e.g., U.S. Pat. No. 7,321,026B2).

Once DNA fragments encoding γc binding protein V_{H} and V_{L} segments are obtained (by, e.g., amplification and mutagenesis of the original murine V_{H} and V_{L} genes, as described above), these DNA fragments can be further manipulated by standard recombinant DNA techniques, for example to convert the variable region genes to full-length antibody chain genes or to Fab fragment genes. In these manipulations, a VL- or VH-encoding DNA fragment is operatively linked to another DNA fragment encoding another protein, such as an antibody constant region or a flexible linker. The term “operatively linked,” as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (CH1, CH2 and CH3). The sequences of human heavy chain constant region genes are known in the art (see e.g., Kabat, E. A., et al (1991) SEQUENCES OF PROTEINS OF IMMUNOLOGICAL INTEREST, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, Ig4, IgA, IgE, IgM or IgD constant region, but most preferably is an IgG1 or IgG4 constant region. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, E. A., et al (1991) SEQUENCES OF PROTEINS OF IMMUNOLOGICAL INTEREST, Fifth Edition, U.S. Department of Health and Human Services, NIH Publication No. 91-3242) and DNA fragments encompassing these regions can be obtained by standard PCR amplification. The light chain const ant region can be a kappa or lambda constant region, but most preferably is a kappa constant region.

To express the γc binding proteins that can be used in the methods disclosed herein, DNAs encoding partial or full-length light and heavy chains, obtained as described above, are inserted into expression vectors (e.g., pEGFP-N1) such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used (e.g., Expi-CHO cell). The antibody light chain gene and the heavy chain gene can be inserted into separate vector (e.g., pEGFP-N1) or, more typically, both genes are inserted into the same expression vector (e.g., pKS1). The antibody genes are inserted into the expression vector by standard methods (e.g., ligation of complementary restriction sites on the antibody gene fragment and vector, or blunt end ligation if no restriction sites are present). In some embodiments, prior to insertion of the protein light or heavy chain sequences, the expression vector already carries sequences encoding heavy chain constant or light chain constant regions, respectively, such that the VH segment is operatively linked to the CH segment (s) within the vector and the VL segment is operatively linked to the CL segment within the vector. Additionally, or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

After transfection of vector containing the full-length antibody sequence into the host cell (e.g., Expi-CHO cell), antibodies were actively secreted into the culture medium and purified using the MabSelect™ PrismA. Antibodies were further buffered exchange into PBS as the storage buffer using the Amicon ultra-15 10k centrifugal filter for higher stability and longer storage duration. If necessary, mouse constant regions are replaced with a desired human constant region, for ex ample wild-type or modified IgG1 or IgG4, to generate a fully human anti-γc antibody. While the constant region selected may vary according to specific use, high affinity antigen-binding and target specificity characteristics reside in the variable region. In certain examples, fully human anti-γc antibodies are isolated directly from antigen-positive B cells from a subject (e.g., a mouse or human).

### Pharmaceutical Compositions

Provided herein are also pharmaceutical compositions comprising the γc binding proteins that can be used in methods disclosed herein. In some embodiments, the pharmaceutical composition comprises a therapeutically effective amount of the γc binding proteins disclosed herein and a pharmaceutically acceptable carrier. In some embodiments, the pharmaceutical compositions are useful in the treatment of immune cell lymphoma and autoimmune diseases. In some embodiments, the pharmaceutical compositions are useful in treating GVHD. In some embodiments, the pharmaceutical compositions are useful in inhibiting GVHD progression in a subject (e.g., a human or a mouse).

The amount of therapeutic γc binding proteins which can be combined with a carrier material in the pharmaceutical compositions disclosed herein can vary. In some embodiments, the amount of γc binding proteins present in the pharmaceutical compositions is the amount that produces a therapeutic effect. Gene rally, out of one hundred percent, this amount will range from about 0.01 percent to about ninety-nine percent of active ingredient, from about 0.1 percent to about 70 percent, or from about 1 percent to about 30 percent of active ingredient in combination with a pharmaceutically acceptable carrier.

The pharmaceutical compositions provided herein comprise the γc binding proteins provided herein. The γc binding proteins can be present at various concentrations. In some embodiments, the pharmaceutical compositions provided herein comprise soluble γc binding proteins provided herein at 1-1000 mg/ml. In some embodiments, the pharmaceutical compositions comprise soluble γc binding proteins provided herein at 10-500 mg/ml, 10-400 mg/ml, 10-300 mg/ml, 10-200 mg/ml, 10-100 mg/ml, 20-100 mg/ml, or 50-100 mg/ml. In some embodiments, the pharmaceutical compositions provided herein comprise the γc binding proteins provided herein at about 10 mg/ml, about 20 mg/ml, about 30 mg/ml, about 40 mg/ml, about 50 mg/ml, about 60 mg/ml, about 70 mg/ml, about 80 mg/ml, about 90 mg/ml, about 100 mg/ml, about 120 mg/ml, about 150 mg/ml, about 180 mg/ml, about 200 mg/ml, about 300 mg/ml, about 500 mg/ml, about 800 mg/ml, or about 1000 mg/ml. Dosages can be readily adjusted by those skilled in the art; for example, a decrease in purity requires an increase in dosage.

The pharmaceutical compositions provided herein can be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Examples of suitable aqueous and nonaqueous carriers that can be employed in the pharmaceutical compositions or formulations described herein include water, ethanol, polyols (such as glycerol, propylene glycol, polyethylene glycol, and the like), and suitable mixtures thereof, vegetable oils, such as olive oil, and injectable organic esters, such as ethyl oleate.

Proper fluidity can be maintained, for example, by the use of coating materials, such as lecithin, by the maintenance of the required particle size in the case of dispersions, and by the use of surfactants. In some embodiments, pharmaceutical compositions provided herein are in the form of injectable or infusible solutions. In some embodiments, the pharmaceutical composition is an aqueous formulation. Such a formulation is typically a solution or a suspension, but can also include colloids, dispersions, emulsions, and multi-phase materials. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water. The composition can be formulated as a solution, microemulsion, liposome, or other ordered structure sui table to high drug concentration.

In some embodiments, the pharmaceutical compositions disclosed herein are freeze-dried, to which the physician or the patient adds solvents and/or diluents prior to use.

The pharmaceutical compositions provided herein can comprise a pharmaceutically acceptable carrier. Pharmaceutically acceptable carriers include any and all solvents, dispersion media, coatings, antibacterial and antifungal agents, isotonic and absorption delaying agents, and the like that are physiologically compatible. Examples include one or more of water, saline, phosphate buffered saline, dextrose, glycerol, ethanol and the like, as well as combinations thereof. In some embodiments, the pharmaceutical acceptable carriers include isotonic agents, for example, sugars, polyalcohols such as mannitol, sorbitol, or sodium chloride in the composition.

In some embodiments, the pharmaceutical acceptable carriers further comprise minor amounts of auxiliary substances such as wetting or emulsifying agents, preservatives or buffers, which enhance the shelf life or effectiveness of the antibody or antigen-binding fragment. In some embodiments, the carrier is suitable for intravenous, intramuscular, subcutaneous, parenteral, spinal or epidermal administration (e.g., by injection or infusion). Depending on the route of administration, the active ingredient (i.e., the γc binding proteins), can be coated in a material to protect the active ingredient from the action of acids and other natural conditions that can inactivate the active ingredient.

Provided herein are also kits for preparation of pharmaceutical compositions having the γc binding proteins disclosed herein. In some embodiments, the kit comprises the γc binding proteins disclosed herein and a pharmaceutically acceptable carrier in one or more containers. In another embodiment, the kits can comprise the γc binding proteins disclosed herein for administration to a subject. In specific embodiments, the kits comprise instructions regarding the preparation and/or administration of the γc binding proteins.

In some embodiments, the pharmaceutical composition or formulation disclosed herein comprises: (a) the γc binding proteins disclosed herein; (b) a buffering agent; (c) a stabilizing agent; (d) a salt; (e) a bulking agent; and/or (f) a surfactant. In some embodiments, the pharmaceutical composition or formulation is stable for at least 1 month, at least 2 months, at least 3 months, at least 6 months, at least 1 year, at least 2 years, at least 3 years, at least 5 years or more. In some embodiments, the pharmaceutical composition or formulation is stable when stored at 4° C., 25° C., or 40° C. In some embodiments, provided herein are also pharmaceutical compositions or formulations that improve the stability of the γc binding proteins to allow for their long-term storage. The pharmaceutical compositions disclosed herein can further comprise one or more of a preservative, a tonicity agent, a chelating agent, a stabilizer and/or a surfactant, as well as various combinations thereof. The use of preservatives, isotonic agents, chelating agents, stabilizers and surfactants in pharmaceutical compositions is well-known to the skilled person. Reference may be made to Remington: THE SCIENCE AND PRACTICE OF PHARMACY, 19th edition, 1995.

Buffering agents useful in the pharmaceutical compositions or formulations disclosed herein can be a weak acid or base used to maintain the acidity (pH) of a solution near a chosen value after the addition of another acid or base. Suitable buffering agents can maximize the stability of the pharmaceutical formulations by maintaining pH control of the formulation. Suitable buffering agents can also ensure physiological compatibility or optimize solubility. Rheology, viscosity and other properties can also depend on the pH of the formulation. Common buffering agents include, but are not limited to, histidine, citrate, succinate, acetate and phosphate. In some embodiments, a buffering agent comprises histidine (e.g., L-histidine) with isotonicity agents and potentially pH adjustment with an acid or a base known in the art. In certain embodiments, the buffering agent is L-histidine. In certain embodiments, the pH of the formulation is maintained between ab out 2 and about 10, or between about 4 and about 8.

Stabilizing agents are added to a pharmaceutical product to stabilize that product. Such agents can stabilize proteins in different ways. Common stabilizing agents include, but are not limited to, amino acids such as glycine, alanine, lysine, arginine, or threonine, carbohydrates such as glucose, sucrose, trehalose, rafftnose, or maltose, polyols such as glycerol, mannitol, sorbitol, cyclodextrins or dextrans of any kind and molecular weight, or PEG. In some embodiments, the stabilizing agent is chosen to maximize the stability of FIX polypeptide in lyophilized preparations. In certain embodiments, the stabilizing agent is sucrose and/or arginine.

Bulking agents can be added to a pharmaceutical composition or formulation to add volume and mass to the product, thereby facilitating precise metering and handling thereof. Common bulking agents include, but are not limited to, lactose, sucrose, glucose, mannitol, sorbitol, calcium carbonate, or magnesium stearate.

Surfactants are amphipathic substances with lyophilic and lyophobic groups. A surfactant can be anionic, cationic, zwitterionic, or nonionic. Examples of nonionic surfactants include, but are not limited to, alkyl ethoxylate, nonylphenol ethoxylate, amine ethoxylate, polyethylene oxide, polypropylene oxide, fatty alcohols such as cetyl alcohol or oleyl alcohol, cocamide MEA, cocamide DEA, polysorbates, or dodecyl dimethylamine oxide. In some embodiments, the surfactant is polysorbate 20 or polysorbate 80.

Pharmaceutical compositions disclosed herein can also include a pharmaceutically acceptable antioxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

These compositions can also contain adjuvants such as preservatives, wetting agents, emulsifying agents and dispersing agents. Prevention of presence of microorganisms can be ensured both by sterilization procedures, supra, and by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin.

Pharmaceutical compositions or formulations typically must be sterile and stable under the conditions of manufacture and storage. Pharmaceutically accept able carriers include sterile aqueous solutions or dispersions and sterile powders for the extemporaneous preparation of sterile injectable solutions or dispersion. Sterile injectable solutions can be prepared by incorporating the therapeutic antibody or antigen-binding fragment in the required amount in an appropriate solvent with one or a combination of ingredients enumerated above, as required, followed by filtered sterilization. The use of such media and agents for pharmaceutically active sub stances is known in the art. Generally, dispersions are prepared by incorporating the active compound into a sterile vehicle that contains a basic dispersion medium and the required other ingredients from those enumerated above. In the case of sterile powders for the preparation of sterile injectable solutions, some methods of preparation are vacuum drying and freeze-drying (lyophilization) that yield a powder of the active ingredient plus any additional desired ingredient from a previously sterile-filtered solution thereof.

The pharmaceutical compositions disclosed herein can be prepared with carriers that protect the active ingredient against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and poly lactic acid. Many methods for the preparation of such formulations are patented or generally known to those skilled in the art. See. e.g., SUSTAINED AND CONTROLLED RELEASE DRUG DELIVERY SYSTEMS, J. R. Robinson, ed., Marcel Dekker, Inc., New York, 1978.

### Treatment and Administration of Anti-γc Protein

In an embodiment of the invention, the anti-γc antibody or/and γc binding protein fragment could be used to treat subject suffering from an γc or γc cytokine-mediated disease or condition, e.g., GVHD, organ transplant rejection, birdshot chorioretinopathy, MS, uveitis, T1D, AD, RA, SLE, asthma, psoriasis, SS, vitiligo, celiac disease, IBD, AA, mast cell mediated diseases, T cell lymphoma, NK cell lymphoma and/or B cell lymphoma.

GVHD refers to the autoimmune condition that might occur after an allogeneic transplant. For example, in GvHD, donated bone marrow or peripheral blood stem cells may regard the recipient's body as foreign, and the donated cells/bone marrow will attack the body of recipient. GvHD may occur, for example, following hematopoietic cell transplantation (HCT; e.g., in a subject suffering from acute myeloid leukemia (AML) or acute lymphocytic leukemia (ALL)) and/or a myelodysplastic syndrome or a myeloproliferative neoplasm), a transfusion, thymus transplantation or in patients with thymoma.

GVHD is less likely to happen when the match between recipient and donor is close. For example, the chance of having GVHD is around 35% to 45% when the donor and recipient are related, while around 60% to 80% when the donor and recipient are not related.

Types of GvHD include steroid-refractory GVHD, acute GVHD and chronic GVHD. Acute GVHD usually happens within days or as late as 6 months after a transplant, while chronic GVHD usually starts more than 3 months after a transplant, and can last a lifetime. One-year overall mortality from date of acute GVHD diagnosis and non-relapse mortality rates are 35.2% and 25.5%, while patients with chronic GVHD tends to exhibit the changes in mouth and skin during recurrence [Holtan et al. Bone Marrow Transplant., 2022; 57, 1581-1585].

Symptoms of acute GVHD include a rash with burning and redness of the skin; nausea, vomiting, abdominal cramps, loss of appetite and diarrhea; jaundice (yellow discoloration of the skin and/or eyes); and/or increased dryness/irritation of the eyes.

Symptoms of chronic GVHD include a very dry mouth; sensitivity to hot, cold, spicy and acidic foods, mint and carbonated drinks; painful mouth ulcers that may extend down the throat; difficulty eating; gum disease and tooth decay; a rash; dry, tight, itchy skin; thickening and tightening of the skin; a change in skin color; intolerance to temperature changes due to damaged sweat glands; changes in nail texture; hard and brittle nails; nail loss; loss of hair on the head; premature gray hair; loss of body hair; loss of appetite, unexplained weight loss, nausea, vomiting diarrhea and stomach pain; shortness of breath and difficulty breathing; a persistent and chronic cough that does not go away; wheezing; abdominal swelling; jaundice; abnormal liver function test results; muscle weakness and cramps; joint stiffness causing difficult full extension of fingers, wrists, elbows, knees or/and ankles; or/and irritative genitalia of women and men.

Common treatments of acute GVHD include increased immunosuppression in the form of corticosteroids and application of JAK inhibitor (e.g., Ruxolitinib). For milder patient with chronic GVHD, close observation or with local/topical therapies using topical steroid ointments are usually applied to them. In addition, recently published manuscripts [Le Floc'h et al., Sci Transl Med., 2023; 15 (678): eabo0205; Le Floc'h et al., Hemasphere. 2022; 6 (Suppl): 694-695] also suggest the use of anti-γc binding protein as the treatment approach.

AA is a disease that happens when the immune system attacks hair follicles and causes hair loss. The cause of AA is not understood and is believed that both genetic and environmental factors (e.g., stress) play a role. People with certain autoimmune diseases, such as psoriasis, thyroid disease, or vitiligo, are more likely to get AA as well.

There are three main types of AA, including patchy alopecia areata (hair loss happens in one or more coin-sized patches on the scalp or other parts of the body), alopecia totalis (lose all or nearly all of the hair on their scalp) and alopecia universalis (complete or nearly complete loss of hair on the scalp, face, and rest of the body).

Generally, symptoms of AA include sudden loss of round or oval patch es of hair on the scalp, or other parts of the body (e.g., beard area in men, or the eyebrows or eyelashes); tingling, burning, or itching feeling on patches of skin right before the hair falls out; or/and nail changes such as ridges and pits. Although AA is not a life-threatening disease, the psychiatric burden of AA may have contributed to increased mortality associated with self-harm, psychiatric diseases, and smoking-associated malignant diseases [Lee et al., JAMA Dermatol. 2019; 155 (8): 922-928].

The common treatments of AA include steroid injection, topical steroids (creams and ointments) and steroid tablets, immunotherapy, dithranol cream, UV light treatment and the use of minoxidil. Besides, recent clinical trials (Identifier NCT03532958, NCT05589610) also suggest the use of anti-γc binding protein as the treatment approach.

The present invention includes clues for treating or preventing those autoimmune diseases (of any kind), in a cell model or a subject (e.g., a mouse), comprising administering a therapeutically effective dosage of an anti-γc protein to the subject.

The term “subject” as used herein refers to any animal (e.g., a mammal), including, but not limited to, humans, non-human primates, canines, felines, rodents, and the like, which is to be the recipient of a particular treatment. A subject can be a human. A subject can be a patient with a particular disease.

As used herein, the term “treat” and its grammatical equivalents in connection with a disease or a condition, or a subject having a disease or a condition refer to an act ion that suppresses, eliminates, reduces, and/or ameliorates a symptom, the severity of the symptom, and/or the frequency of the symptom associated with the disease or disorder being treated. For example, when used in reference to GVHD, the term “treat” and its grammatical equivalents refer to an action that reduces the severity of the disease, or retards or slows the progression of the disease, including, but not limited to (a) absence of secondary systemic treatment, or (b) absence of nonrelapse mortality, or (c) absence of recurrent or progressive malignancy [Martin et al., Blood., 2017; 130 (3): 360-367].

The term “block” and its grammatical equivalents refer to an action that reduces the biological function of alarmin in a way including but not limited to (a) directly competing for the binding site of alarmin on its corresponding receptors, or (b) preventing heterodimerization of corresponding receptors, reducing biological effects induced by alarmin occupancy.

As used herein, the term “administer” and its grammatical equivalents refer to the act of delivering, or causing to be delivered, a therapeutic or a pharmaceutical composition to the body of a subject by a method described herein or otherwise known in the art. The therapeutic can be a compound, a polypeptide, or a cell. Administering a therapeutic or a pharmaceutical composition includes prescribing a therapeutic or a pharmaceutical composition to be delivered into the body of a subject. Exemplary forms of administration include oral dosage forms, such as tablets, capsules, syrups, suspensions; injectable dosage forms, such as intravenous (IV), intramuscular (IM), or intraperitoneal (IP); subcutaneous (SC), transdermal dosage forms, including creams, jellies, powders, or patches; buccal dosage forms; inhalation powders, sprays, suspensions, and rectal suppositories.

As used herein, the terms “effective amount,” “therapeutically effective amount,” and their grammatical equivalents refer to the administration of an agent to a subject, either alone or as a part of a pharmaceutical composition and either in a single do se or as part of a series of doses, in an amount that is capable of having any detect able, positive effect on any symptom, aspect, or characteristics of a disease, disorder or condition when administered to the subject. The therapeutically effective amount can be ascertained by measuring relevant physiological effects. The exact amount required varies from subject to subject, depending on the age, weight, and general condition of the subject, the severity of the condition being treated, the judgment of the clinician, and the like. A therapeutically effective amount is also one in which any toxic or detrimental effects of the therapeutic agent are outweighed by the therapeutically beneficial effects. An appropriate “effective amount” in any individual case can vary according to factors such as the disease state, age, sex, and weight of the individual, and can be determined by one of ordinary skill in the art using routine experimentation. A “prophylactically effective amount” refers to an amount effective, at dosages and for periods of time necessary, to achieve the desired prophylactic result, for example, the delay or prevention of the onset of a disease or disorder. Typically, since a prophylactic dose is used in subjects prior to or at an earlier stage of disease, the prophylactically effective amount is commonly less than the therapeutically effective amount.

As used herein and understood in the art, “EC” means effective concentration of an agent (e.g., antibody), and is commonly used in dose-response curves. The “effect” of the agent can be a positive (activatory) effect or a negative effect. The term “EC50” refers to the concentration of an active agent (e.g., antibody) that gives half-maximal response. Also as used herein and understood in the art, “IC” means concentration of an agent that has an inhibitory effect and is also commonly used for dose-response curves. The term “IC50” refers to the concentration of an agent (e.g., antibody) where the activity that it inhibits is reduced by half.

An effective or therapeutically effective dose of anti-γc protein, e.g., antibody or anti gen-binding fragment, for treating or preventing an γc-mediated disease or condition refers to the amount of the antigen-binding protein sufficient to alleviate one or more signs and/or symptoms of the disease or condition in the treated subject, whether by inducing the regression or elimination of such signs and/or symptoms or by inhibiting the progression of such signs and/or symptoms. In an embodiment of the invention, an effective or therapeutically effective dose of anti-γc protein is about 0.05-50 mg/kg of body weight. The dose amount may vary depending upon the age and the size of a subject to be administered, target disease, conditions, route of administration, and the like. In certain embodiments, the initial dose may be followed by administration of a second or a plurality of subsequent doses of antigen-binding protein in an amount that can be approximately the same or 1 ess or more than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

Prevention of γc-mediated disease or condition refers, as it relates to use of an anti-γc protein of the present invention, to administration to a subject prior to manifestation of the disease or condition in the body of the subject so as to stop such manifestation from occurring.

### Immunoconjugates

The invention encompasses anti-γc proteins, e.g., antibodies or antigen-binding fragments, conjugated to another moiety, e.g., a therapeutic moiety (an “immunoconjugate”). In an embodiment of the invention, an anti-γc protein, e.g., antibody or antigen-binding fragment, is conjugated to any of the furth er therapeutic agents set forth herein. As used herein, the term “immunoconjugate” refers to an antigen-binding protein, e.g., an antibody or antigen-binding fragment, which is chemically or biologically linked to another antigen-binding protein, a drug, a radioactive agent, a reporter moiety, an enzyme, a peptide, a protein or a therapeutic agent.

### Effects and Properties of Anti-γc Protein

The anti-γc proteins (either chimeric or humanized version) set forth herein, e.g., comprising variant immunoglobulin chains, may exhibit one or more of the following properties:
Binds to human γc (e.g., a fusion thereof such as a 6×His tags (SEQ ID NO: 234) or murine Fc) at 25° C., with an indicated K_{D} as listed in Table 11;
Binds to common marmoset γc (e.g., a fusion thereof such as a 6×His tags (SEQ ID NO: 234)) at 25° C., with an indicated K_{D} as listed in Table 12;
Partially binds to mouse γc (e.g., a fusion thereof such as a 6×His tags (SEQ ID NO: 234)) at 25° C., with an indicated K_{D} as listed in Table 12;
Partially binds to rat γc (e.g., a fusion thereof such as a 6×His tags (SEQ ID NO: 234)) at 25° C., with an indicated K_{D} as listed in Table 12;
Does not bind to cynomolgus γc (e.g., a fusion thereof such as a 6×His tags (SEQ ID NO: 234)) at 25° C., with an indicated K_{D} as listed in Table 12;
Maintains high structural stability at 37° C. within 2 weeks;
Demonstrates no ADCC and CDC activities to γc expressing cell lines;
Suppresses STAT phosphorylation in cell lines including NK cell culture (e.g., human NK cell line KHYG-1), B cell culture (e.g., human Bcell line Ramos), T cell culture (e.g., human T cell lines HPB-ALL and Jurkat) induced by six γc cytokines, for example, as measured by the luciferase expression in the cell including a luciferase gene operably linked to the STAT3/STAT5/STAT6 response elements or endogenous expression of phosphorylated STAT3/STAT5/STAT6 proteins;
Blocks STAT phosphorylation in primary PBMC culture induced by six γc cytokines (except IL-9);
Blocks IL-4 and IL-21 rescues of anti-IgM induced cell death and damage in B cell culture (e.g., human B cell line Ramos):
Blocks IL-4 and IL-21 induced CD23 and PRDM1 expression in B cell culture (e.g., human B cell line Ramos);
Inhibits cell proliferation in human immune cells (e.g., PBMC culture, primary T cell, primary NK cell, KHYG-1 cell) induced by IL-2, IL-7, IL-15 or IL-21;
Suppresses expression of survival marker BCL-2 of HPB-ALL T cells induced by IL-7;
Suppresses CD127 internalization and degradation in HPB-ALL T cells induced by IL-7;
Attenuates IL-9 induced protection and proliferation of Jurkat T cells in the presence of C2-Ceramide;
Inhibits ERK phosphorylation in Jurkat T cells induced by IL-9;
Inhibits secretion of soluble factors (e.g., proinflammatory cytokines and cytotoxic enzymes) from NK cell or PBMC culture induced by IL-2, IL-15 or IL-21;
Suppresses expression of activating receptors on the surface of cytotoxic T and NK cells in the PBMC culture;
Attenuates MLR induced by allograft primary T cells;
Binds to the same epitope of human γc, for example, on the coated γc proteins in the ELISA strip as any one or more than one γc binding proteins set forth herein;
Competes for binding to human γc, for example, on the coated γc proteins in the ELISA strip as any one or more than one γc binding proteins set forth herein.

### EXAMPLES

### Experimental Examples

The following examples are put forth so as to provide those of ordinary skill in the art with a complete disclosure and description of how to make and use the methods and compositions of the invention and are not intended to limit the scope of what the inventors regard as their invention.

### Example 1: Development and Generation of Anti-γc Antibodies

Anti-γc antibodies were obtained from the phage display library after ani mal immunization with the human γc proteins (Sequence ID: NP_000197.1) set forth in SEQ ID NO: 205, composing of the extracellular domain of human γc protein (amino acids: 1-262) and murine Fc tag (underlined amino acids: 278-509) linked with three repeats of G₄S linker (SEQ ID NO: 236) (amino acids: 263-277), or with the human γc proteins set forth in SEQ ID NO: 206, composing of the extracellular domain of human γc protein (amino acids: 1-262) and 6×His tag (SEQ ID NO: 234) (underlined amino acids: 263-268).

Anti-γc ScFv was first constructed using HB2151 bacterial host cell system to evaluate the specificity and inhibitory features of anti-γc antibodies. ELISA was used to evaluate the binding activities of constructed anti-γc ScFv to human γc protein. Briefly, in-house generated human γc proteins were diluted to 1 μg/ml in PBS and 100 μl of γc proteins were added to each well of ELISA strip. The strips were sealed with parafilm and incubated at 4° C. overnight for coating. On the next day, strips were washed thrice with washing buffer (0.05% Tween 20 in PBS) and blocked with 100 μl per well with blocking buffer (3% BSA in PBS) for 2 hours RT before 10 μg/ml anti-γc ScFv was added. After an incubation period of 2 hrs at RT, the amount of bound anti-γc ScFv were revealed by the addition of peroxidase conjugated goat anti-human F (ab′) 2 specific antibody (Jackson ImmunoResearch, West Grove, USA) and TMB substrate (Sigma-Aldrich, St. Louis, MO) at OD 450 nM following standard ELISA protocol known to those skilled in the art. Results indicates that all purified anti-γc ScFvs bind to γc proteins from human when compared to PBS and non-transfected TG1 controls at 25° C. and 37° C. (FIG. 1).

Full-length chimeric anti-γc antibodies were generated using the Expi-CHO host cell and purified by standardized method. Each antibody consists of four polypeptides: two heavy immunoglobulin chains and two light immunoglobulin chains joined to form a “Y” shaped molecule. Each heavy chain consists of a V_{H} chain inked to a heavy chain constant region, while each light chain consists of a V_{L} chain inked to a light chain constant region. Each antibody belongs to either one of five well known classes of antibodies, IgA (IgA and IgA2), IgD, IgE, IgG (IgG1, IgG 2, IgG3 and IgG4) and IgM, respectively. Three signal peptides were tested in product ion strategy. Signal peptides 2 and 3 provide a better production yield than signal peptide 1. The stability of purified antibodies was determined by sodium dodecyl sulfate-polyacrylamide gel electrophoresis (SDS-PAGE) using the 10% non-reducing gel, showing that all purified antibodies are intact except SM-05E (FIG. 2).

### Example 2: Flow Cytometry Analyses of p-STAT Using Anti-γc ScFv in Multiple Cell Lines

Flow cytometry was employed to measure the percentage of p-STAT expressing cells after cytokine induction and anti-γc ScFv treatment. KHYG-1 (Creative Bioarray, Shirley, NY 11967, USA) was starved in IL-2 free RPMI1640 (ATCC modification, ThermoFisher Scientific, Waltham, MA, USA) with 10% FBS (ThermoFisher Scientific) for 2 days. Ramos, HPB-ALL and Jurkat cell lines were used directly without starvation. Cells were replenished with fresh RPMI1640 medium and pretreated with anti-γc ScFv (10 μg/mL) for an hour. After treatment with TL-4 (10 ng/ml, Sino Biological), IL-7 (50 ng/ml, Sino Biological), IL-9 (50 ng/ml, Sino Biological), IL-15 (10 ng/ml, Sino Biological) or IL-21 (10 ng/ml, Sino Biological) for 15 min, cells were fixed in 4% paraformaldehyde (PFA, Sigma, St. Louis, Missouri, USA) for 10 min, permeabilized in 0.1% Triton X-100 (Sigma) for 10 min and then stained with FITC conjugated anti-pSTAT6, PE conjugated anti-pSTAT3 or APC con jugated anti-pSTAT5 (all diluted in 1:100, Biolegend, San Diego, California, USA) for 30 min at 37° C. Percentage of p-STAT expressing cells was measured by BD FACSLyric™ Clinical Flow Cytometry System (BD Biosciences, New Jersey, USA). Data were analyzed by Flowjo (version 10, BD).

Different cell lines were used for testing. KHYG-1 refers to the NK cell line harvested from the peripheral blood of a 45-year-old woman with aggressive NK cell leukemia. This cell line could respond to IL-2 and IL-15 stimulation in-vitro [Yamasaki et al., Leuk Res., 2014; 28 (10): 1023-31]. Ramos is a human Burkitts Lymphoma cell line (CRL-1596™) and could be used as a model for IL-4 and IL-21 studies [Hui et al., Front Immunol., 2022; 13: 919854]. Jurkat is a human T lymphoblast cell line (Clone E6-1, TIB-152™) and is widely used in the T cell studies of IL-9 function. HPB-ALL is a T cell leukemia model established from the peripheral blood of a 14-year-old Japanese boy with ALL and thymoma at diagnosis in 1973 and could be used for IL-7 functional studies.

Results indicate that four clones (SM-05A, SM-05E and SM-05F) potentially suppress a highest combination of γc cytokine mediated p-STAT expression, thus processing to the generation of full-length antibodies (Tables 7-10).

### Example 3: EC50 of the Purified Full-Length Anti-γc Antibodies

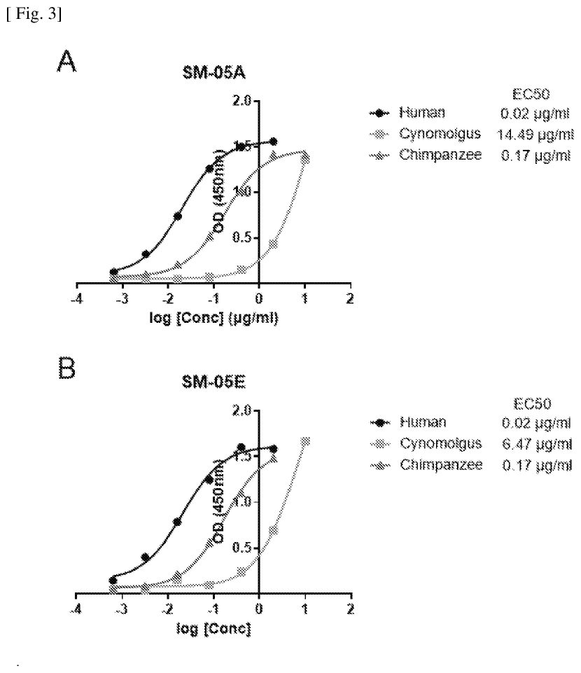

Selected clones of full-length antibodies were evaluated by ELISA to access the binding specificity. The binding activities of full-length anti-γc antibody were investigated using γc proteins (extracellular domain) from human (SEQ ID NO: 205-206), cynomolgus (Sequence ID: XP_005593949.1, SEQ ID NO: 207-208) and chimpanzee (Sequence ID: XP_008971810.1, SEQ ID NO: 209-210) either purchased from Sino Biological or in-house generated. Results indicate that anti-γc antibodies bind to γc proteins from human and chimpanzee with com parable affinity in a dose-response manner but show low/no binding affinity to cynomolgus γc proteins (FIG. 3).

### Example 4: Flow Cytometry Analyses of p-STAT Using Full-Length Anti-γc Antibodies in Multiple Cell Lines

Flow cytometry was employed to measure the percentage of p-STAT expressing cells after cytokine induction and anti-γc antibody treatment as previously described. IL-2 and IL-15 were tested in KHYG-1 NK cell line; IL-4 and IL-21 were tested in Ramos B cell line; IL-7 was tested in HPB-ALL T cell line; and IL-9 was tested in Jurkat T cell line.

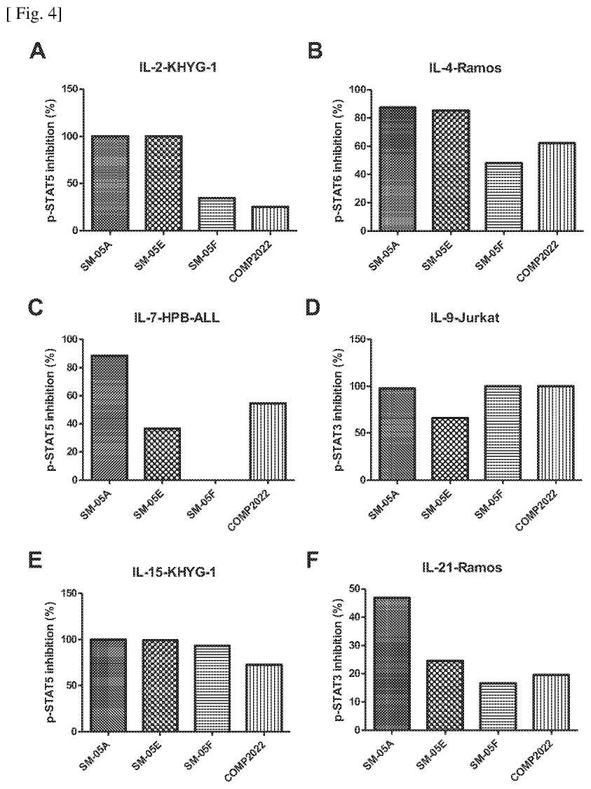

Both SM-05A and SM-05E show comparable efficacies to block STAT phosphorylation induced by IL-2 in KHYG-1 NK cell (FIG. 4A), IL-4 in Ramos B cell (FIG. 4B), IL-7 in HPB-ALL T cell (FIG. 4C), IL-9 in Jurkat T cel 1 (FIG. 4D), IL-15 in KHYG-1 NK cell (FIG. 4E) and IL-21 in Ramos cell (FIG. 4F), with a comparable efficacy as the competitor antibody COMP2022 (sequence from WO2020160242A1). SM-05F barely demonstrated strong inhibitory effects to STAT phosphorylation induced by 6 γc cytokines.

### Example 5: Bioassays for B Cell Studies

Ramos could be used as a model for B-cell antigen receptor (BCR) activation by cross-linking the BCR with anti-IgM antibody, which would lead to the induction of cell cycle arrest and apoptosis.

The effects of anti-γc antibodies in blocking IL-4 and IL-21 rescues of Ramos cell death after anti-IgM induced hyperactivation were investigated by WST-8 proliferation assay. In brief, Ramos cells (50000 cells in 100 l) were seeded into 96-well plate and stabilized at 37° C. for an hour. Cells were pretreated in anti-γc antibodies or ritlecitinib for an hour, followed by incubation of IL-4 (10 ng/ml) or IL-21 (10 ng/ml) and anti-IgM (2 ug/ml, #109-006-129, Jackson ImmunoResearch). Aft er 48 hours, 10 μl WST-8 reagent (ab228554, Abcam) was added to each well and incubated for 4 hours.

WST-8 could be reduced by cellular dehydrogenases to an orange formazan product, which the amount of formazan produced is directly proportional to the number of living Ramos cells. Optical density (OD) of each well was measured by absorbance at 450 nm using Varioskan LUX Multimode Microplate Reader (ThermoFisher Scientific).

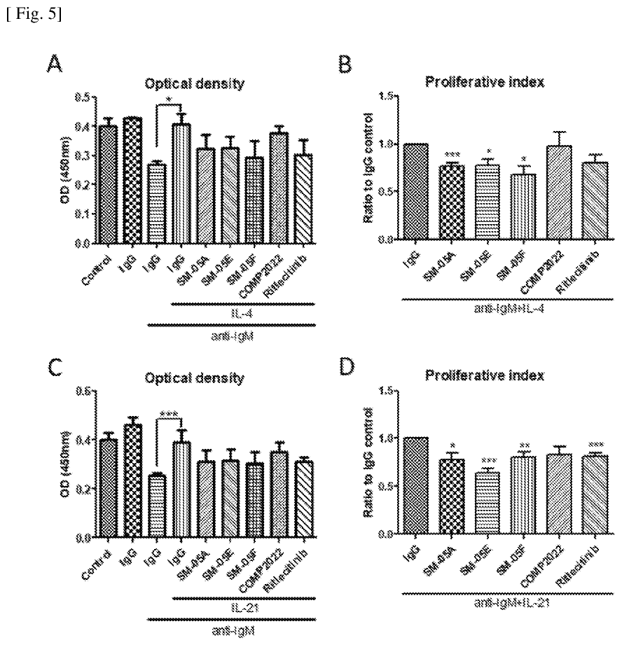

WST-8 assay confirms that IL-4 and IL-21 could rescue anti-IgM induced cell death in Ramos B cells, while the application of anti-γc antibodies (SM-05A, SM-05E, SM-05F) could block the effects of IL-4 and IL-21. The overall effects of anti-γc antibodies in the present invention are better than COMP2022 and ritlecitinib when applied at the same molarity (33 nM) (FIG. 5A-D).

The above effects could be explained by apoptotic assay using the de ad cell apoptosis kits with annexin V (AV) for flow cytometry (#V132 42, ThermoFisher Scientific). In brief, cells were treated with the same paradigm and then stained with AV/propidium iodide (PI) according to manufacturer's protocol. Number of apoptotic cells (AV+PI− and AV+PI+ cells) was analyzed by Flowjo.

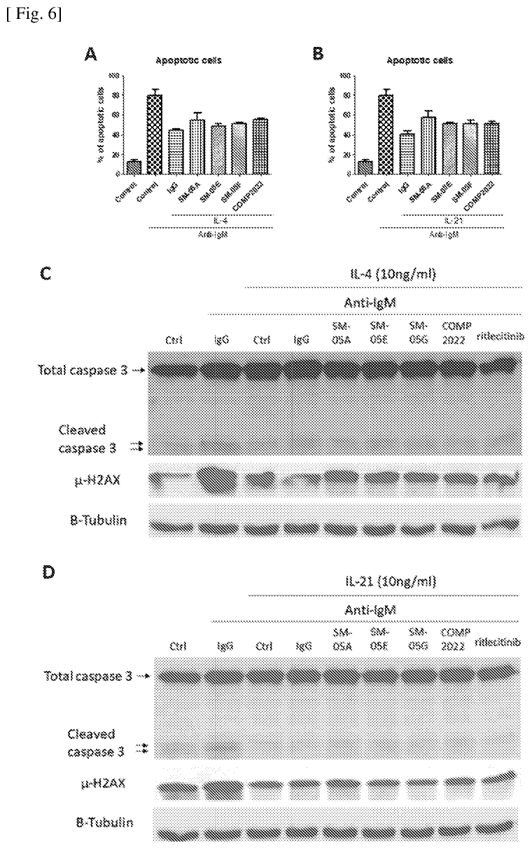

All tested anti-γc antibodies partially reverse the IL-4 and IL-21 rescues of anti-IgM induced apoptosis in Ramos B cells, with the strongest effect observed in the group treated with SM-05A (FIG. 6A-B).

Levels of cleaved caspase 3 (apoptotic protein) and μ-H2AX (DNA damage marker) were investigated by western blot in the Ramos B cells treated in the same paradigm. In brief, total proteins were extracted from the Ramos cells in RIPA lysis buffer supplemented with Halt™ Protease and Phosphatase Inhibitor Cocktail. Proteins were denatured and separated through electrophoresis and blotted on the nitrocellulose membrane. The membrane was blocked with 5% nonfat milk diluted in TBST, then incubated with primary antibodies diluted in 5% BSA/TBS T at 4° C. overnight in a rotating wheel. Next day, membrane was washed with PBST and incubated with secondary antibodies diluted in 5% milk/TBST for an hour at RT. Intensities of protein bands were determined using ECL substrate kit in the ChemiDoc Imaging System.

Consistent to the results in WST-8 and AV/PI assays, IL-4 and IL-21 could rescue anti-IgM induced apoptotic and DNA damage markers. Application of anti-γc antibodies and ritlecitinib could block IL-4 protective effects and the level of μ-H2AX is re-induced, while SM-05A shows the strongest efficacy to re-induce cleaved caspase 3 and μ-H2AX in the presence of IL-21 (FIG. 6C-D).

Genes related to the B cell functions were investigated by quantitative real-time PCR (qRT-PCR). In brief, Ramos cells were starved in serum-free RPMI1640 medium for 24 hours and then treated with 10 ng/ml IL-4 or IL-21 for 24 hours. Total RNA was extracted by RNAzol following manufacturer's protocol. Reverse transcription was performed in 500 ng of total RNA using PrimeScript RT reagent kit and the gene expression was analyzed by quantitative real-time PCR using TB Green Premix Ex Taq in Lightcycler® 480 Real-Time PCR System. Housekeeping gene GAPDH was used for normalization.

CD23 and PRDM1 expressions are investigated in Ramos after IL-4 or IL-21 treatment. CD23 is a low affinity receptor for IgE expressed on the surface of activated B cells and classic memory B cells. It enhances the antibody response, promotes the production of IgE in its soluble form, inhibits the production of membrane IgE, and controls the proliferation and apoptosis of B cells [Veneri et al., Blood Transfus., 2009; 7 (1): 29-34].

PRDM1 is an important transcriptional factor in B cells. It controls the terminal differentiation of antibody-secreting cells (ASCs) and IL-10 producing regulatory B cells (Bregs) [Nutt et al., Nat Rev Immunol., 2007; 7 (12): 923-7; Wang et al., Front Immunol., 2019; 10: 1909].

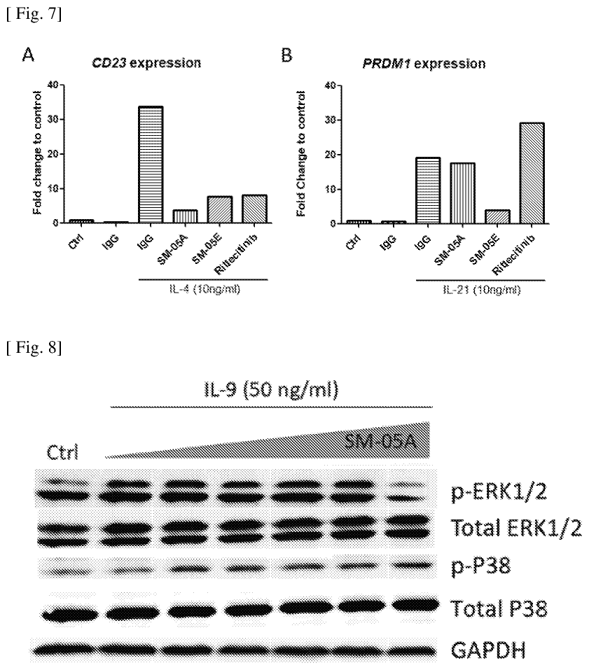

Results demonstrate that SM-05A, SM-05E and ritlecitinib significantly suppress IL-4 induced CD23 expression, while only SM-05E inhibits IL-21 induced PRDM1 expression (FIG. 7).

### Example 6: Bioassays for T Cell Studies

IL-9 receptor is expressed on surface of Jurkat cells and could be manipulated for studying relationship between IL-9 and autoimmune diseases/tumor bi ology [Lv et al., J Exp Clin Cancer Res., 2016; 35 (1): 106].

Western blot demonstrated that IL-9 could phosphorylate pro-survival ERK signaling pathway and application of SM-05A could block this (FIG. 8).

IL-7 has been shown to induce STAT5 phosphorylation and is responsible in cell growth, proliferation and survival of HPB-ALL cells [Ribeiro et al., Blood Adv., 2018; 2 (17): 2199-2213], suggesting that HPB-ALL is a reliable cell line to study IL-7 function.

Several assays, such as were proposed to study cell survival of HPB-ALL from literature [Ribeiro et al., Blood Adv., 2018; 2 (17): 2199-2213] was measured in terms of BCL2 (anti-apoptotic marker) expressing cells by flow cytometry. In brief, HPB-ALL was pre-treated with antibodies or inhibitors for an hour before stimulated with 50 ng/ml IL-7 for 3 days. Cells were harvested and stained with FITC-conjugated BCL-2 antibody (Biolegend) for flow cytometry analysis.

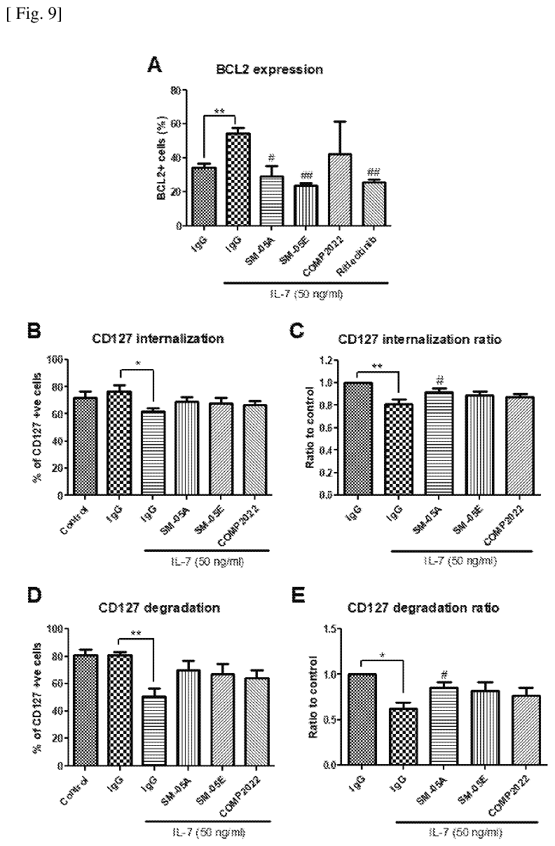

Results indicate that both SM-05A and SM-05E significantly attenuate IL-7 induced BCL-2 expression comparable to ritlecitinib treated group (FIG. 9A). Competitive antibody COMP2022 shows variability in this assay.

Specific methods for IL-7 function were developed in HPB-ALL [Henriques et al., Blood., 2010; 115 (16): 3269-77]. IL-7 could trigger the internalization of its own receptor, CD127 (or IL-7Rai), into the cell compartment for degradation. As the CD127 degradation depends on the JAK3 activated status after IL-7 stimulation, blockade of γc binding to CD127 and downstream signaling pathway should stop CD127 internalization and degradation in HPB-ALL.

After antibody or inhibitor pretreatment, HPB-ALL was challenged with 50 ng/ml IL-7 for 3 hours. Surface CD127 expression was performed in live cells while total CD127 expression was investigated in PFA fixed and permeabilized cells. Cells were stained with APC-conjugated CD127 antibody for flow cytometry analysis.

Results indicate that all tested anti-γc antibodies could block IL-7 induced CD127 internalization and degradation in HPB-ALL cells, with the highest potency observed in cells treated with SM-05A (FIG. 9B-E).

### Example 7: Bioassays for NK Cell Studies

The effects of anti-γc antibodies in NK cell homeostasis were studied in the KHYG-1 cell line (CSC-C0784, Creative Bioarray), as established from the peripheral blood of a 45-year-old woman with aggressive NK leukemia. As reported in literature, KH YG-1 could respond to IL-2 and IL-15 stimulation while no information could be retrieved for other γc cytokines. We tested the responsiveness of KHYG-1 to the γc cytokines before the antibody testing.

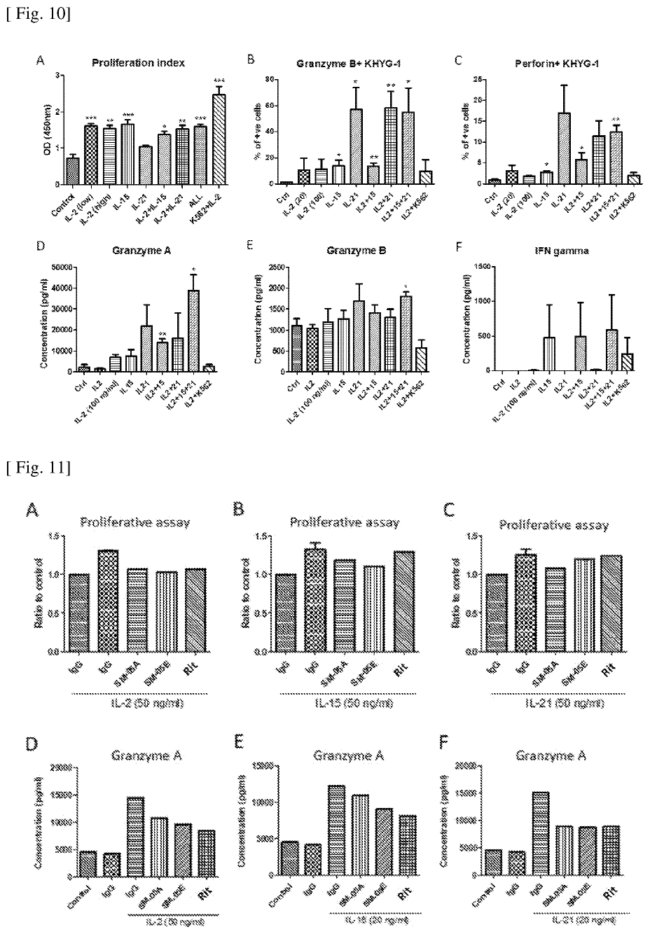

Proliferation assay was performed in the overnight starved KHYG-1 cells with the incubation of different combination of γc cytokines for 3 days. Results demonstrate that KHYG-1 cells could respond to IL-2 and IL-15 to proliferate, while no synergistic effect is observed among all combination groups (FIG. 10A).

Production of cytotoxic factors is monitored by the flow cytometry in KHYG-1 cells after 3-day cytokine incubation. Cells were fixed in 4% PFA and then permeabilized for intracellular staining with pacific blue conjugated granzyme B and APC conjugated perforin antibodies (Biolegend). Results demonstrate that IL-2, IL-15 and IL-21 could increase the percentage of granzyme B/perforin expressing cells after 3 days, while the strongest effect is observed for the groups with IL-21 treatment (FIG. 10B-C).

The results obtained from flow cytometry are consistent with the ELI SA results obtained in the supernatant. IL-21 serves as the main γc cytokine to induce the production and secretion of granzymes and perforin from KHYG-1 cells (FIG. 10D-E). Different results are observed for the IFNγ ELISA that IL-15 is the main γc cytokine to induce IFNγ secretion in KHYG-1 cells (FIG. 10F). All ELISA assays were performed in commercially available kits (R&D systems, Minneapolis, USA).

To test the efficacies of anti-γc antibodies, proliferation and granzyme A secretion were chosen as the experimental models. In brief, KHYG-1 was seeded into either 48- or 96-well plates and starved in non-IL-2 supplemented medium overnight. After replenishment with new medium, KHYG-1 was pre-incubated with anti-γc antibodies or ritlecitinib for an hour before the administration of γc cytokines for 3 days. Proliferation rate was determined by the addition of WST-8 reagent while the secretion of granzyme A was estimated by ELISA measurement.

Results demonstrate that both SM-05A and SM-05E could suppress IL-2, IL-15 and IL-21 induced NK cell proliferation, as compared to the groups treated with JAK3 inhibitor ritlecitinib (FIG. 11A-C).

Different results were observed in granzyme A secretion that SM-05E and ritlecitinib significantly suppress IL-2, IL-15 and IL-21 induced granzyme A secretion in NK cell culture, with SM-05A providing fewer effects in the suppression (FIG. 11D-F).

### Example 8: Bioassays for Ex-Vivo PBMC Culture

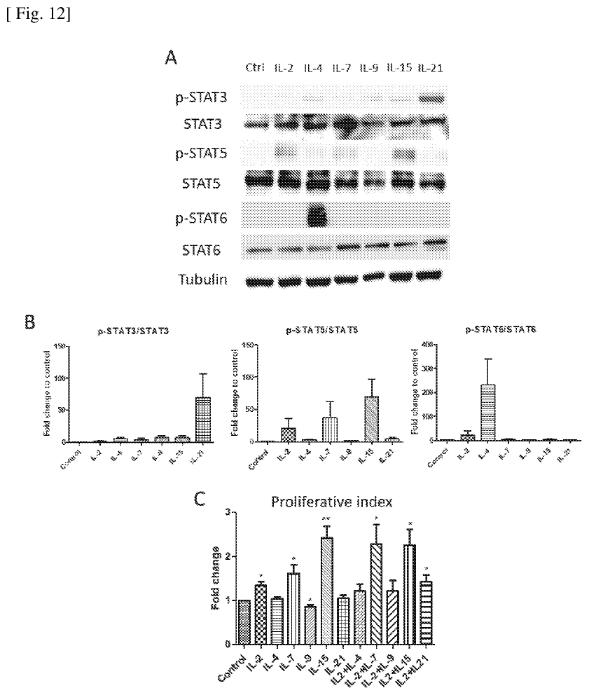

PBMC isolated from healthy individuals (iXCells Biotechnologies, San Diego, USA) is chosen as the primary immune cell culture to conclude the efficacies of anti-γc antibodies in the primary cells. As STAT phosphorylation could be triggered by the treatment of all six γc cytokines (except IL-9) in the PBMC culture (FIG. 12A-B), inhibition of p-STAT could be used as an indicator to confirm the efficacies of anti-γc antibodies.

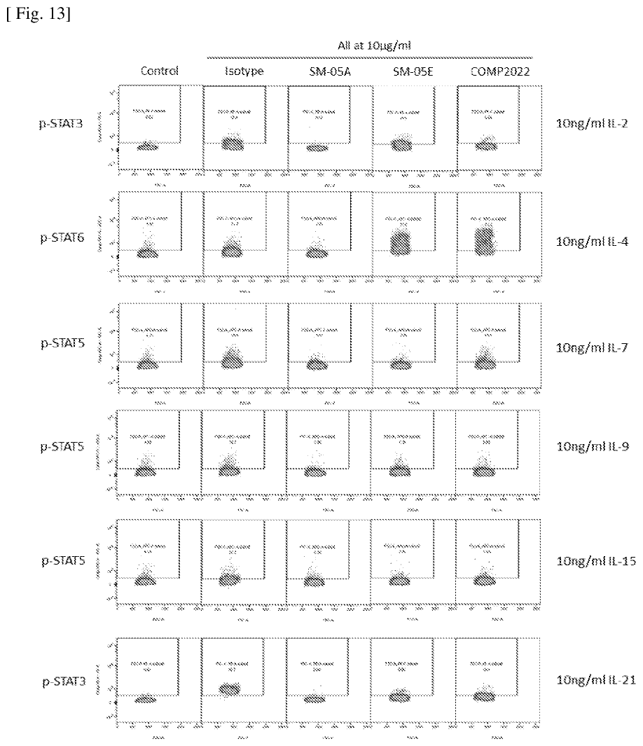

SM-05A remains as the strongest candidate to suppress STAT phosphorylation induced by all six γc cytokines in the PBMC culture, while SM-05E and COMP2022 failed to suppress IL-4 induced p-STAT6 in the PBMC culture (FIG. 13).

Proliferation was also investigated in PBMC culture following the similar protocol established for KHYG-1 NK cell line and the results demonstrate that PBMC could mainly respond to IL-2, IL-7 and IL-15 in terms of proliferation (FIG. 12C). After antibody and cytokine incubation for 3 days, WST-8 reagent was add ed into the wells and the proliferation was measured in terms of the signal intensity measured at 450 nm using microplate reader.

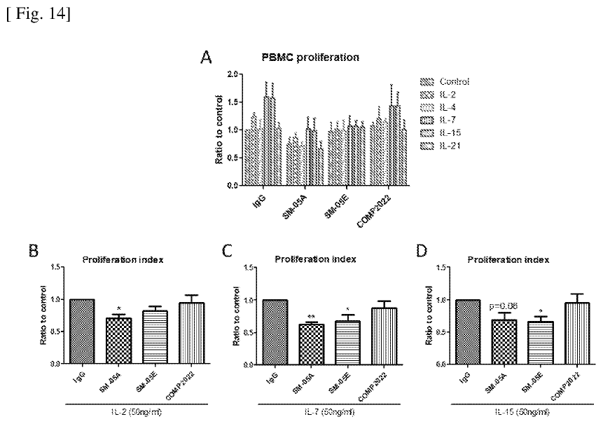

The obvious inhibitory effect in proliferation is shown in groups with SM-05A treatment (FIG. 14A). Further quantification confirms both SM-05A and SM-05E could suppress proliferation induced by IL-2, IL-7 and IL-15, while SM-05A provides a slightly stronger effect. Competitor antibody COMP2022 barely shows anti-proliferative effect in PBMC culture (FIG. 14B-D).

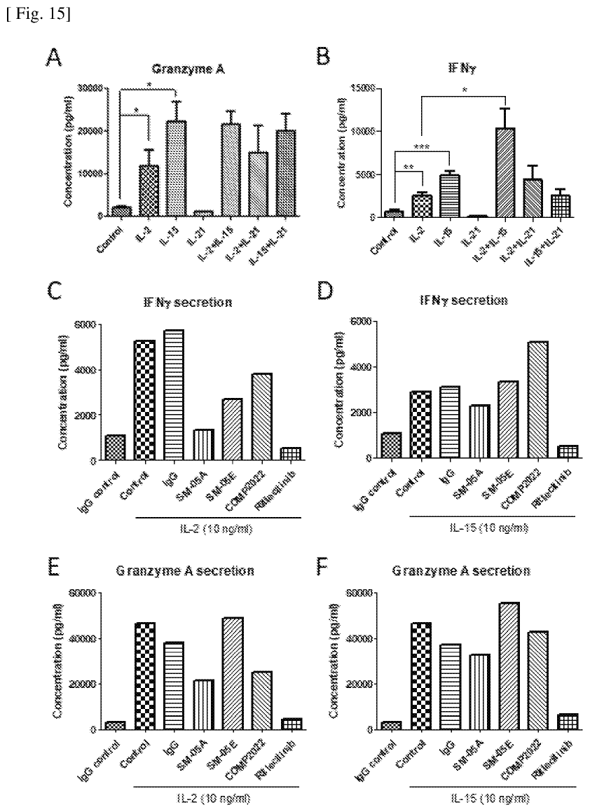

Activation of T and NK cells in the PBMC culture could be studied by granzyme and IFNγ secretion [Carlin et al., Blood., 2005; 106 (12): 3874-9; Grossman et al., Blood., 2004; 104 (9): 2840-8; Keppel et al., J Immunol., 2015; 194 (4): 1954-1962; Tamang et al., Cytokine., 2006; 36 (3-4): 148-59]. Similar results are obtained in-house that IL-2 and IL-15 but not IL-21 could trigger secretion of granzyme A and IFNγ in PBMC culture (FIG. 15A-B).

Antibodies or ritlecitinib were added into the PBMC culture to investigate their inhibitory effects in the presence of IL-2 or IL-15 treatment. Similar to previous results, SM-05A remains as the strongest anti-γc antibody to suppress IL-2 and IL-15 induced secretion of granzyme A and IFNγ, while the level of both soluble factors is significantly reduced in ritlecitinib than the control group which suggest the potential toxicity of the JAK3 inhibitor (FIG. 15C-F).

Suppression of T cell activation was further evaluated by expression of receptors related to cell death. One of the examples refers to Fas ligand (FasL), which is mainly expressed on activated T cells and could trigger apoptosis in the Fas-expressing cells upon binding [García-González and Selvi, Encyclopedia of Medical Immunology, 2014; pp 413-416].

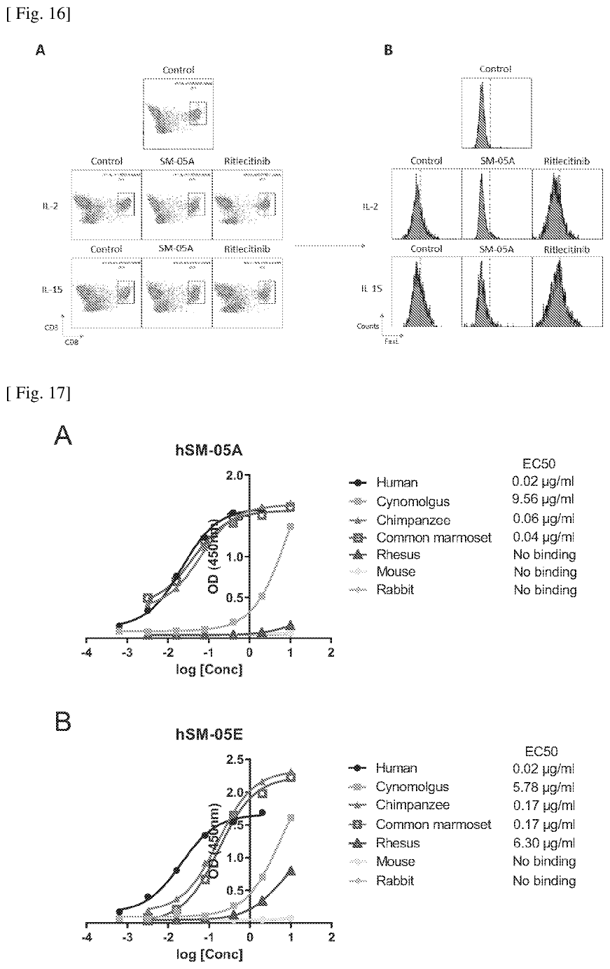

Although treatment with either IL-2 or IL-15 could only slightly increase the cytotoxic T cell population (CD3+CD8+), both cytokines could significantly induce FasL expression on the gated cytotoxic T cell population. Compared to the ritlecitinib treated groups, SM-05A could significantly reduce cytokine induced Fas L expression on cytotoxic T cell (FIG. 16).

### Example 9: Humanization of Anti-γc Antibodies

From the preliminary results obtained from cell lines and primary cultures, both SM-05A and SM-05E serve as good candidates to suppress the γc cytokine induced STAT phosphorylation and cellular functions. Thus, humanization of both anti-γc proteins was performed using framework patching method (see e.g., U.S. Pat. No. 7,321,026B2) for future clinical uses.

Humanized sequences for heavy and light variable chains are shown in SEQ ID NO:193-200. In some embodiments, humanized antibodies were maintained in fully humanized version. In some embodiments, back-mutation in certain amino acids in the framework regions was introduced to the humanized antibodies to improve the biological activities. The humanized variable heavy and variable light domains are linked to heavy and light constant regions of human origin set forth in SEQ ID NO: 201-202 or SEQ ID NO: 203-204.

Humanized antibodies with back-mutation (hSM-05A and hSM-05E) were tested in the following assays. Binding affinities were investigated in the ELISA assays. Results indicate that both hSM-05A and hSM-05E retained the binding affinity and specificity to the human and chimpanzee γc protein (FIG. 17).

Additional extracellular domains of γc proteins from common marmoset (Sequence ID: NP_001288775.1, SEQ ID NO: 211-212), rhesus (Sequence ID: NP_001030606.1, SEQ ID NO: 213-214), rabbit (Sequence ID: XP_008 270982.1, SEQ ID NO: 215-216), mouse (Sequence ID: NP_038591.1, SEQ ID NO: 217-218) and rat (Sequence ID: NP_543165.1, SEQ ID NO: 219-220) were either purchased from Sino Biological or in-house generated. Results indicate that common marmoset is another biologically relevant species to both γc antibodies, while no binding affinities were observed to other studied species (FIG. 17).

### Example 10: Stability and Binding Kinetics of Anti-γc Antibodies

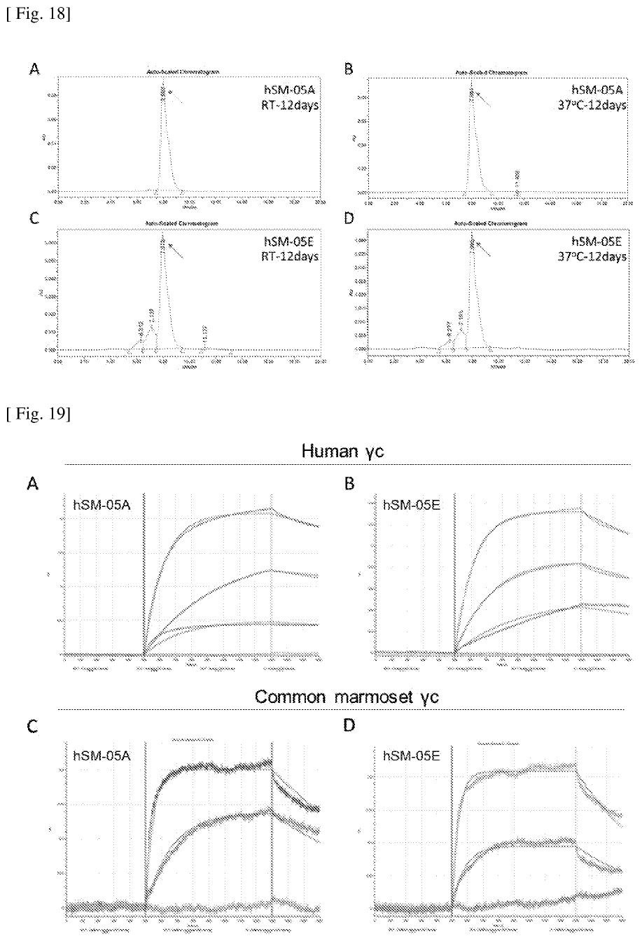

Next, structural stabilities of hSM-05A and hSM-05E were studied by incubation at either room temperature or 37° C. for around 2 weeks. SEC-HPLC was utilized to measure aggregates and degradation of antibodies. Studies demonstrated that hSM-05A remained as single molecule, while around 20% of hSM-05E formed aggregates after incubation (FIG. 18). No degradation of the antibodies was observed aft er 2 weeks.

Full-length humanized antibodies were evaluated by BLI to access their binding kinetics. The binding activities of full-length anti-γc antibody were investigated using γc proteins (extracellular domain) from human and common marmoset (either purchased or in-house generated). Briefly, anti-γc antibody (20 μg/mL) was immobilized on biosensors via interactions with anti-human IgG; serially diluted γc proteins (at a concentration of 158.7 nM, 79.4 Nm, 39.7n M and 0 nM respectively) were subsequently added following standard operation protocols of the Octet ReD96 system to plot out the association and dissociation curve. Irrelevant antibody (SM03, anti-human CD22 chimeric IgG1 antibody, SinoMab BioScience Limited) was used as the control reference. R square, Kₐ, K_{dis} and K_{D} values of the respective antibodies are summarized in Table 11. The K_{D} is within the picomolar-single digit nanomolar range. Results indicate that anti-γc antibodies bind to γc proteins from human and common marmoset with comparable binding kinetics (FIG. 19 and Table 11-12).

TABLE 11
Binding Kinetics of hSM-05A and hSM-05E to
human γc proteins from different species

| | Experiment 1 | | | Experiment 2 | | |
|---|---|---|---|---|---|---|
| Human | Kₐ (1/Ms) | K_{dis} (1/s) | K_{D} (nM) | Kₐ (1/Ms) | K_{dis} (1/s) | K_{D} (nM) |
| hSM-05A | 1.19 × 10⁵ | 3.19 × 10⁻⁴ | 2.69 | 1.75 × 10⁵ | 1.44 × 10⁻³ | 8.23 |
| hSM-05E | 1.19 × 10⁵ | 5.73 × 10⁻⁴ | 4.82 | 3.27 × 10⁵ | 9.44 × 10⁻⁴ | 2.89 |
| SM03 | N/A | N/A | N/A | N/A | N/A | N/A |

TABLE 12
Binding Kinetics of hSM-05A and hSM-
05E to γc proteins from other species

| | Kₐ (1/Ms) | K_{dis} (1/s) | K_{D} (nM) |
|---|---|---|---|
| Common marmoset | | | |
| hSM-05A | 3 × 10⁵ | 1.21 × 10⁻³ | 4.05 |
| hSM-05E | 3.33 × 10⁵ | 1.75 × 10⁻³ | 5.27 |
| Cynomolgus | | | |
| hSM-05A | 2.25 × 10⁴ | 6.83 × 10⁻³ | 304 |
| hSM-05E | 2.25 × 10⁴ | 6.83 × 10⁻³ | 304 |
| Mouse | | | |
| hSM-05A | 6.58 × 10³ | 7.12 × 10⁻⁴ | 108 |
| hSM-05E | 3.4 × 10⁵ | 2.52 × 10⁻² | 66.2 |
| Rat | | | |
| hSM-05A | 3.28 × 10⁴ | 7.20 × 10⁻⁵ | 16.1 |
| hSM-05E | 7.1 × 10⁴ | 2.08 × 10⁻³ | 29.3 |

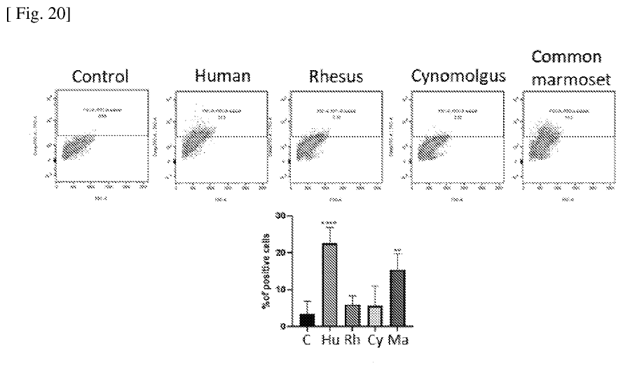

The binding specificities of humanized anti-γc antibodies to native γc proteins were further evaluated by in-vivo binding assay. In brief, human HEK-293 cells were transfected with plasmid expressing full length γc protein from human, rhesus, cynomolgus or common marmoset. Two days later, transfected cells or non-transfected controls were incubated with FITC-conjugated hSM-05A on ice for an hour before analysis by flow cytometry. Results demonstrated that h SM-05A could specifically target native γc proteins from human and comm on marmoset expressing on the HEK-293 cells (FIG. 20).

### Example 11: Competition Assays Between Anti-γc Antibodies

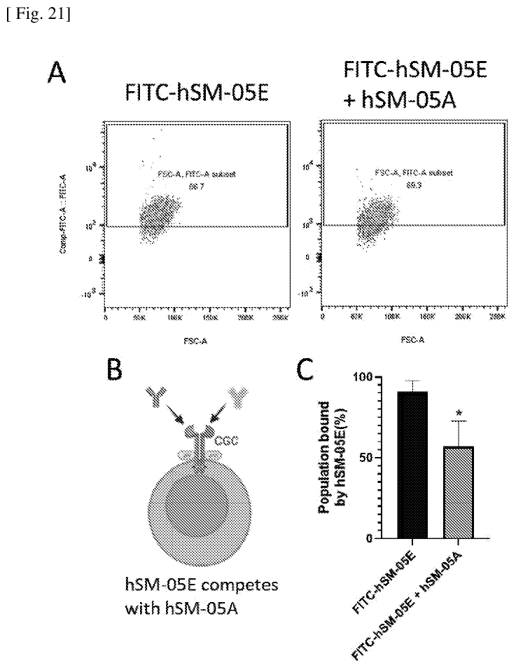

Purified anti-γc antibodies were conjugated to FITC fluorescent dye and used for the competition assay. In brief, human γc expressing Ramos B cells were first labelled with unconjugated hSM-05A for an hour before further incubation with FITC-conjugated hSM-05E. Flow cytometry analyses demonstrated that two anti-γc antibodies could compete on a similar epitope on the human γc protein (FIG. 21).

### Example 12: Functional Assays for ADCC/CDC Activity Determination

As an immune-modulatory antibody to block the activation rather than cell death induction in immune cells, ADCC and CDC need to be avoided in γc binding proteins to prevent excessive killing of immune cells or cytokine release from several effector cells.

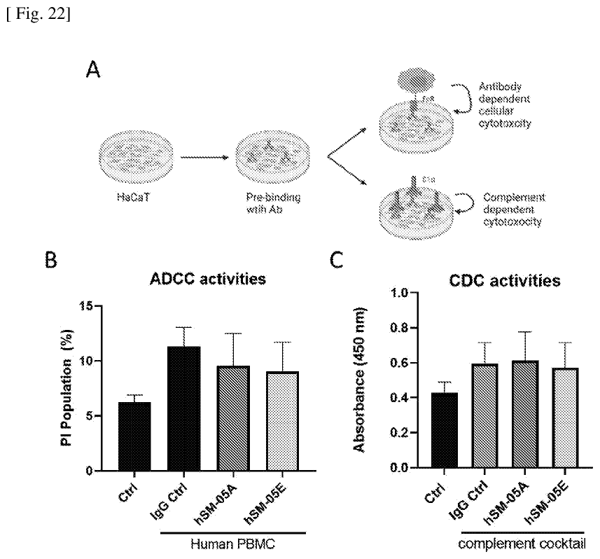

γc expressing human keratinocyte HaCaT cell line was utilized as the target cells in ADCC and CDC assays. To determine the ADCC activity, HaCaT cell was pre-binded with anti-γc antibodies before co-culture with human PBMC. To determine the CDC activity, HaCaT cell was pre-binded with anti-γc antibodies before incubation with low-endotoxin complement proteins extracted from guinea pig. ADCC was determined by percentage of PI+ HaCaT cells in flow cytometry analysis while CDC was measured by the WST-8 proliferative assay of HaCaT cells in a 96-well plate (FIG. 22A).

Quantification clearly demonstrated that both hSM-05A and hSM-05E could not induce any ADCC nor CDC activities in the HaCaT cells when compared to the isotype antibody treated group. Those assays provided evidence for safety uses of anti-γc antibodies to future therapeutic investigations.

### Example 13: Functional Assays for Immuno-Modulatory Effects of Humanized Antibodies

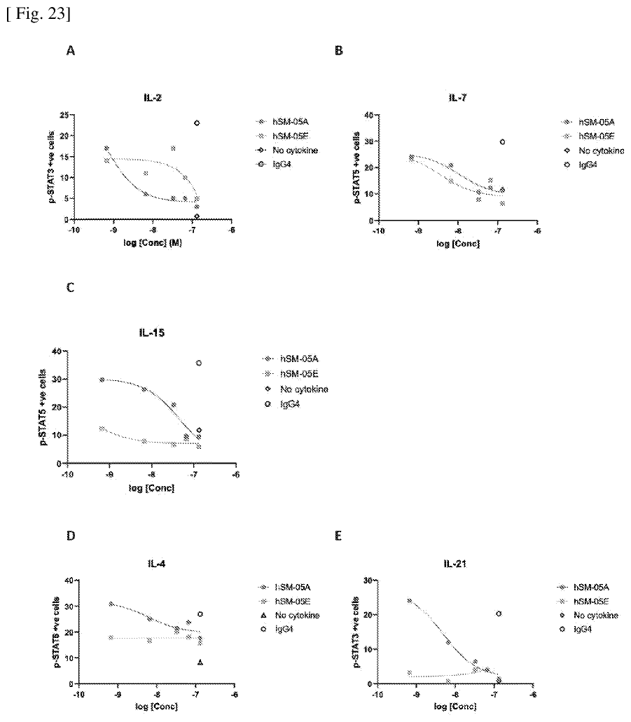

STAT phosphorylation analyses were performed again to study the potencies of humanized anti-γc proteins as shown in Table 13. Results demonstrate that the both versions of humanized antibodies could show comparable p-STAT inhibitory activities when compared to chimeric versions (FIG. 23).

TABLE 13
Suppression of p-STAT in PBMC by humanized anti-γc antibodies

| | IL-2 | IL-4 | IL-7 | IL-15 | IL-21 |
|---|---|---|---|---|---|
| | induced | induced | induced | induced | induced |
| | p-STAT3 | p-STAT6 | p-STAT5 | p-STAT5 | p-STAT3 |
| hbSM-05A | 89.94% | 17.18% | 94.17% | 100% | 82.80% |
| hbSM-05E | 58.45% | 21.74% | 66.02% | 100% | 100% |

Given numbers refer to the percentage of inhibition compared to isotype control stimulated with respective γc cytokine. Antibody concentration is 10 μg/ml.

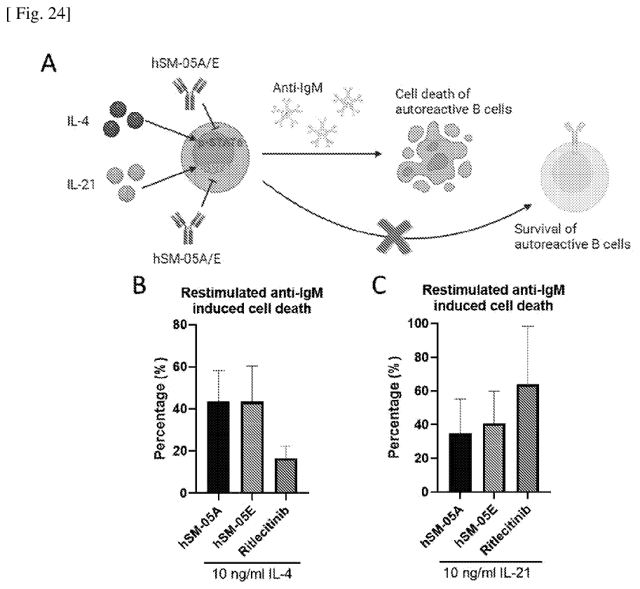

Immuno-modulatory effects of humanized antibodies were re-tested in Ramos B cells with a WST-8 proliferative assay. Results showed that both antibodies could pro vide better inhibition to IL-4 but not IL-21 effects than ritlecitinib (FIG. 24).

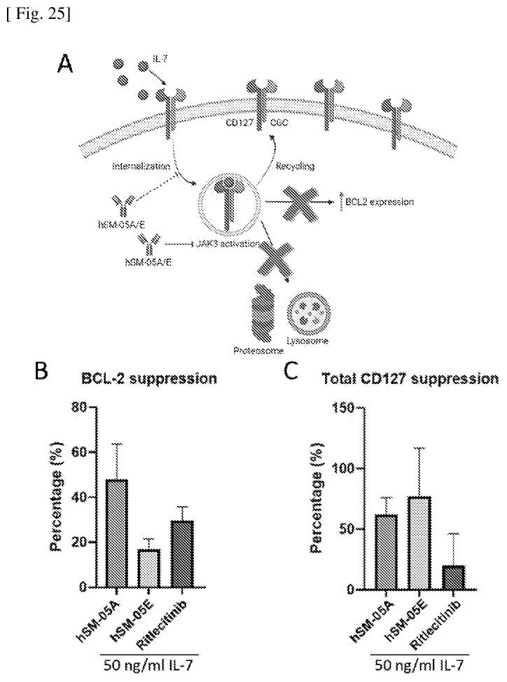

In HPB-ALL T cells, hSM-05A could provide better suppression to IL-7 induced BCL-2 upregulation and CD127 degradation than ritlecitinib (FIG. 25).

Previous studies have shown that IL-9 could rescue the anti-tumor drug driven apoptosis in the B cell line model of diffuse large-B-cell lymphoma (DL BCL) [Lv et al., J Exp Clin Cancer Res., 2016; 35 (1): 106]. Similar experiments we re performed on Jurkat T cells to evaluate the efficacies of anti-γc antibodies.

Jurkat T cells were pretreated with anti-γc antibodies or ritlecitinib before the application of anti-tumor ceramide (50 μM) and IL-9 (50 ng/ml) for three days. To evaluate cell survival, 10 μl WST-8 reagent was added to each well and incubated for 4 hours before spectrophotometer measurement.

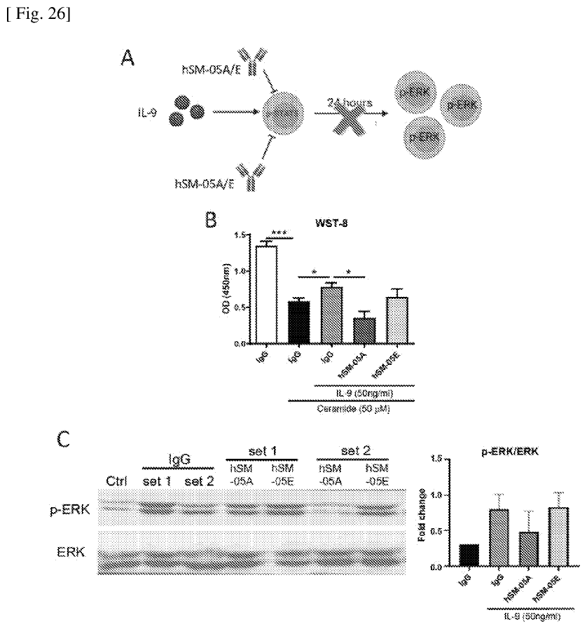

Results indicate that IL-9 could rescue Jurkat cell death in the presence of ceramide while the application of hSM-05A could suppress the IL-9 effect (FIG. 26B). Furthermore, hSM-05A could provide better suppression to IL-9 induced ERK phosphorylation than hSM-05E (FIG. 26C), indicating that hSM-05A could suppress T cell survival and proliferation in the presence of IL-9.

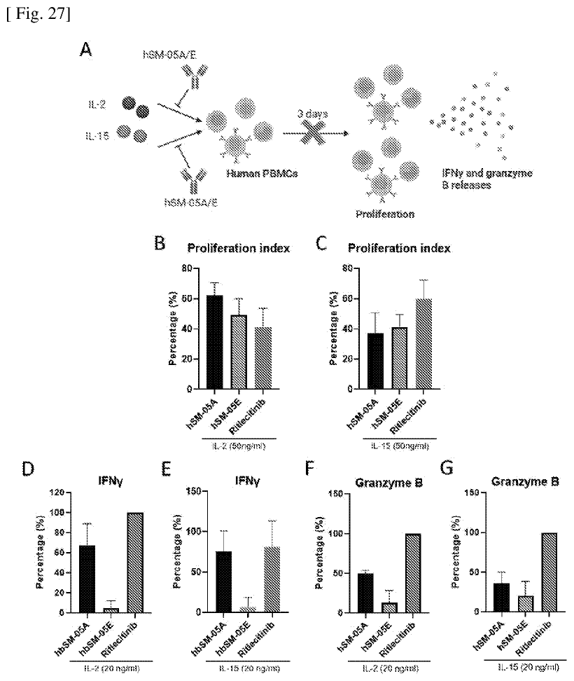

In human PBMC culture, the effects of IL-2 and IL-15 in cell proliferation were determined by WST-8 proliferative assays. Results showed that both antibodies could provide better inhibition to IL-2 but not IL-15 effects than ritlecitinib (FIG. 27B-C).

Releases of cytolytic agents from PBMC culture were further evaluated by ELISA assays. Although hSM-05A could provide comparable inhibition to IFNγ secretion to ritlecitinib, ritlecitinib could offer superior suppression to granzyme B secretion in the culture (FIG. 27D-G). hSM-05E only offered weak suppression to two agents.

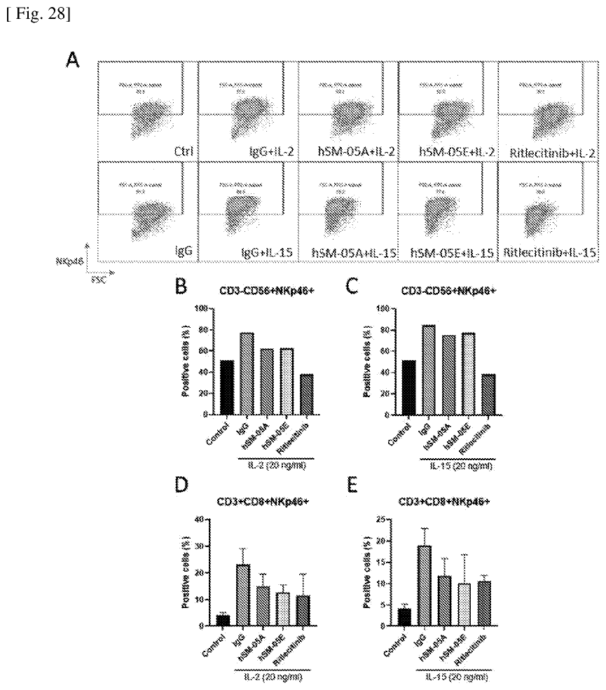

Activation of NK cells and cytotoxic T cells was investigated by the expression of activating receptor NKp46 on their surfaces. PBMC was pretreated with humanized antibodies or ritlecitinib for an hour before incubation with IL-2 or IL-15 for 3 days. NK cells were gated as CD3-CD56+ population while cytotoxic Tcells were identified as CD3+CD8+ population under flow cytometry analyses. Quantification showed that both humanized antibodies were effective in suppressing IL-2 and IL-15 induced activation of cytotoxic T cells while only provided mild inhibition to NK cell activation as compared to ritlecitinib treated groups (FIG. 28).

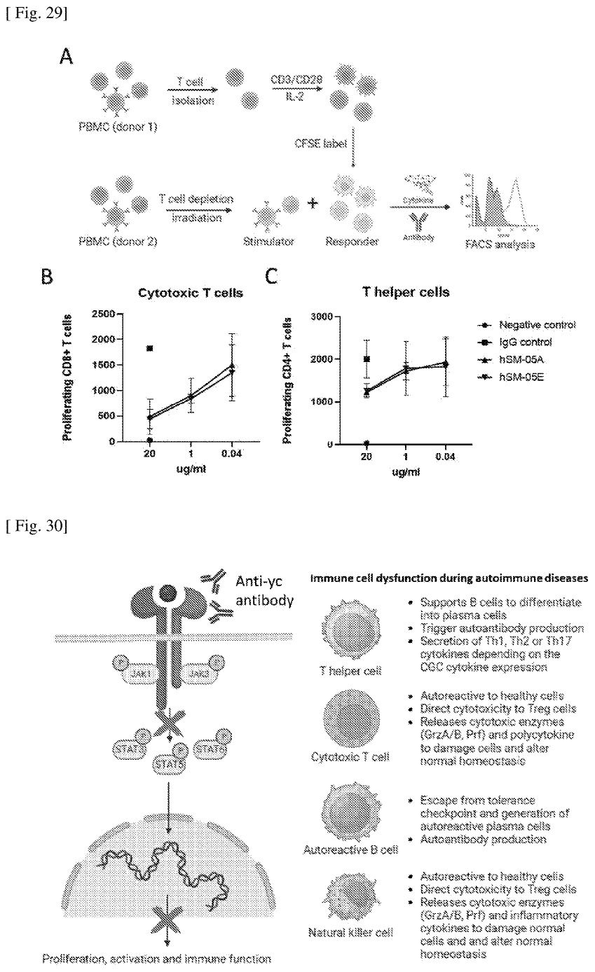

To provide hints at effects of humanized antibodies to target GvHD, MLR assay was developed in our PBMC system (FIG. 29A). In brief, total T cells were isolated from PBMC of donor 1 while T cells were depleted from PBMC of donor 2. T cells from donor 1 were activated with a cocktail of anti-CD3 and anti-CD28 antibodies in the presence of IL-2. Simultaneously, T cells were incubated with humanized antibodies or ritlecitinib together with inducing agents. After 3 days, T cells were labelled with CFSE before co-culture with T-cell depleted culture from donor 2 for 6 days. Proliferating T cells were identified as CFSE low or CSFE negative population in flow cytometry.

Results indicated that both hSM-05A and hSM-05E could significantly inhibit ML R induced expansion of cytotoxic T cells in a dose dependent manner, while the suppression was milder to T helper cells (FIG. 29B-C).

### RESULTS SUMMARY AND CONCLUSION

The current invention describes the developmental procedures of anti-γc binding proteins and provides the DNA/amino acid sequences for the developed anti-γc binding proteins. Those proteins suppressed γc related downstream pathways (JAK/STAT) and biological functions (survival, proliferation, activation, cytokine secretion, cytotoxic enzyme secretion, differentiation, receptor internalization, cytotoxicity) in multiple immune cell types, including but not limited to B cell, CD4+T helper cell, CD8+ cytotoxic T cell, NK cell, mast cell, in the single cell or PBMC cultures (FIG. 30). The current invention is suggested to show therapeutic potential in the treatment of treating an γc or γc cytokine mediated disease or condition (e.g., GVHD, organ transplant rejection, birdshot chorioretinopathy, MS, uveitis, T1D, AD, RA, SLE, asthma, psoriasis, SS, vitiligo, celiac disease, IBD, AA, mast cell mediated diseases, T cell lymphoma, NK cell lymphoma and/or B cell lymphoma), in a subject in need thereof, comprising administering, e.g., injecting, an effective amount of antigen-binding protein or composition set forth herein.

1. An isolated antigen-binding protein that specifically binds to common gamma chain (γc) or an antigenic fragment thereof, wherein said antigen-binding protein exhibits one or more of the following features:
(a) binding to human γc or a fusion thereof with K_{D} lower than 10⁻⁹M;
(b) binding to chimpanzee and common marmoset γc or a fusion thereof with K_{D} lower than 10⁻⁹M;
(c) partially binding to mouse and rat γc or a fusion thereof with K_{D} at 10⁻⁸M;
(d) not binding detectably to rhesus, cynomolgus and rabbit γc or a fusion thereof;
(e) suppressing STAT phosphorylation induced by IL-4 and IL-21 in Ramos B cells;
(f) suppressing STAT phosphorylation induced by IL-7 in HPB-ALL T cells;
(g) suppressing STAT phosphorylation induced by IL-9 in Jurkat T cells;
(h) suppressing STAT phosphorylation induced by IL-2 and IL-15 in KHYG NK cells;
(i) suppressing STAT phosphorylation induced by IL-2, IL-4, IL-7, IL-9, IL-15 or IL-21 in PBMC culture;
(j) blocking IL-4 and IL-21 rescues of anti-IgM induced cell death and damage in B cell culture;
(k) blocking IL-4 and IL-21 inducible genes in B cell culture;
(l) inhibiting cell proliferation in human immune cells, including NK cell, T cell and PBMC culture;
(m) attenuating IL-9 induced protection and proliferation of Jurkat T cells in the presence of anti-tumor ceremide;
(n) inhibiting secretion of proinflammatory cytokines and cytotoxic enzymes from NK cell or/and PBMC cultures;
(o) inhibiting activation of NK and T cells in the primary immune cell culture;
(p) specifically binding to a same epitope on γc as a reference antibody or antigen-binding fragment thereof, wherein the reference antibody or antigen-binding fragment thereof is a chimeric or humanized SM-05A, SM-05B, SM-05C, SM-05D, SM-05E, SM-05F, SM-05G, SM-05H, SM-05I, SM05J, SM-05K or SM-05L;
(q) competing for binding to a γc polypeptide or antigenic fragment thereof with a reference antibody or antigen-binding fragment thereof, wherein the reference antibody or antigen-binding fragment thereof is a chimeric or humanized SM-05A, SM-05B, SM-05C, SM-05D, SM-05E, SM-05F, SM-05G, SM-05H, SM-05I, SM05J, SM-05K or SM-05L; and
(r) blocking binding of heterodimer composing of γc and respective γc cytokine receptor subunit in the presence of IL-2, IL-4, IL-7, IL-9, IL-15 or IL-21.

2. The isolated antigen-binding protein of claim 1, wherein said isolated antigen-binding protein is an isolated antibody or an antigen-binding fragment of an antibody.

3. (canceled)

4. The isolated antigen-binding protein of claim 2, wherein the isolated antibody or antigen-binding fragment comprises:
(a) a heavy chain or a heavy chain variable region, that comprises CDR-H1, CDR-H2 and CDR-H3 comprised in the amino acid sequence set forth in SEQ ID NO: 2, 18, 34, 50, 66, 82, 98, 114, 130, 146, 162, 178, or a variant thereof; and/or,
(b) a light chain or a light chain variable region, that comprises CDR-L1, CDR-L2 and CDR-L3 comprised in the amino acid sequence set forth in SEQ ID NO: 10, 26, 42, 58, 74, 90, 106, 122, 138, 154, 170, 186 or a variant thereof.

5. The isolated antigen-binding protein of claim 2, wherein the isolated antibody or antigen-binding fragment comprises:
(a) a heavy chain or a heavy chain variable region thereof comprising an amino acid sequence having at least 90% amino acid sequence identity to the amino acid sequence set forth in SEQ ID NO: 2, 18, 34, 50, 66, 82, 98, 114, 130, 146, 162 or 178; and/or,
(b) a light chain or a light chain variable region thereof comprising an amino acid sequence having at least 90% amino acid sequence identity to the amino acid sequence set forth in SEQ ID NO: 10, 26, 42, 58, 74, 90, 106, 122, 138, 154, 170 or 186.

6. The isolated antigen-binding protein of claim 4, wherein the isolated antibody or antigen-binding fragment comprises:
(a) a heavy chain or a heavy chain variable region thereof, comprising the CDR-H1, CDR-H2 and CDR-H3 comprised in an amino acid sequence set forth in SEQ ID NO: 2, 18, 34, 50, 66, 82, 98, 114, 130, 146, 162 or 178 and comprising an amino acid sequence having at least 90% amino acid sequence identity to the amino acid sequence set forth in SEQ ID NO: 2, 18, 34, 50, 66, 82, 98, 114, 130, 146, 162 or 178; and/or,
(b) a light chain or a light chain variable region thereof, comprising the CDR-L1, CDR-L2 and CDR-L3 comprised in an amino acid sequence set forth in SEQ ID NO: 10, 26, 42, 58, 74, 90, 106, 122, 138, 154, 170 or 186 and comprising an amino acid sequence having at least 90% amino acid sequence identity to the amino acid sequence set forth in SEQ ID NO: 10, 26, 42, 58, 74, 90, 106, 122, 138, 154, 170 or 186.

7. The isolated antigen-binding protein of claim 4, wherein the isolated antibody or antigen-binding fragment comprises:
(a) a heavy chain or heavy chain variable region thereof comprising CDR-H1, CDR-H2 and CDR-H3 set forth in:
SEQ ID NO:4, SEQ ID NO:6, and SEQ ID NO:8, respectively;
SEQ ID NO:20, SEQ ID NO:22, and SEQ ID NO:24, respectively;
SEQ ID NO:36, SEQ ID NO:38, and SEQ ID NO:40, respectively;
SEQ ID NO:52, SEQ ID NO:54, and SEQ ID NO:56, respectively;
SEQ ID NO:68, SEQ ID NO:70, and SEQ ID NO:72, respectively;
SEQ ID NO:84, SEQ ID NO:86, and SEQ ID NO:88, respectively;
SEQ ID NO:100, SEQ ID NO:102, and SEQ ID NO:104, respectively;
SEQ ID NO: 116, SEQ ID NO: 118, and SEQ ID NO:120, respectively;
SEQ ID NO:132, SEQ ID NO:134, and SEQ ID NO:136, respectively;
SEQ ID NO:148, SEQ ID NO:150, and SEQ ID NO:152, respectively;
SEQ ID NO:164, SEQ ID NO:166, and SEQ ID NO:168, respectively; or
SEQ ID NO:180, SEQ ID NO:182, and SEQ ID NO:184, respectively; and/or,
(b) a light chain or variable region thereof comprising CDR-L1, CDR-L2 and CDR-L3 set forth in:
SEQ ID NO:12, SEQ ID NO:14, and SEQ ID NO:16, respectively;
SEQ ID NO:28, SEQ ID NO:30, and SEQ ID NO:32, respectively;
SEQ ID NO:44, SEQ ID NO:46, and SEQ ID NO:48, respectively;
SEQ ID NO:60, SEQ ID NO:62, and SEQ ID NO:64, respectively;
SEQ ID NO:76, SEQ ID NO:78, and SEQ ID NO:80, respectively;
SEQ ID NO:92, SEQ ID NO:94, and SEQ ID NO:96, respectively;
SEQ ID NO:108, SEQ ID NO:110, and SEQ ID NO:112, respectively;
SEQ ID NO:124, SEQ ID NO:126, and SEQ ID NO:128, respectively;
SEQ ID NO:140, SEQ ID NO:142, and SEQ ID NO:144, respectively;
SEQ ID NO:156, SEQ ID NO:158, and SEQ ID NO:160, respectively;
SEQ ID NO:172, SEQ ID NO:174, and SEQ ID NO:176, respectively; or
SEQ ID NO:188, SEQ ID NO:190, and SEQ ID NO:192, respectively.

8. The isolated antigen-binding protein of claim 7, wherein the isolated antibody or antigen-binding fragment comprises CDR-H1, CDR-H2, CDR-H3, CDR-L1, CDR-L2 and CDR-L3 set forth in:
SEQ ID NO:4, SEQ ID NO:6, SEQ ID NO:8, SEQ ID NO:12, SEQ ID NO:14, and SEQ ID NO:16, respectively;
SEQ ID NO:20, SEQ ID NO:22, SEQ ID NO:24, SEQ ID NO:28, SEQ ID NO:30, and SEQ ID NO:32, respectively;
SEQ ID NO:36, SEQ ID NO:38, SEQ ID NO:40, SEQ ID NO:44, SEQ ID NO:46, and SEQ ID NO:48, respectively;
SEQ ID NO:52, SEQ ID NO:54, SEQ ID NO:56, SEQ ID NO:60, SEQ ID NO:62, and SEQ ID NO:64, respectively;
SEQ ID NO:68, SEQ ID NO:70, SEQ ID NO:72, SEQ ID NO:76, SEQ ID NO:78, and SEQ ID NO:80, respectively;
SEQ ID NO:84, SEQ ID NO:86, SEQ ID NO:88, SEQ ID NO:92, SEQ ID NO:94, and SEQ ID NO:96, respectively;
SEQ ID NO:100, SEQ ID NO:102, SEQ ID NO:104, SEQ ID NO:108, SEQ ID NO:110, and SEQ ID NO:112, respectively;
SEQ ID NO: 116, SEQ ID NO: 118, SEQ ID NO: 120, SEQ ID NO:124, SEQ ID NO:126, and SEQ ID NO:128, respectively;
SEQ ID NO:132, SEQ ID NO:134, SEQ ID NO: 136, SEQ ID NO:140, SEQ ID NO:142, and SEQ ID NO:144, respectively;
SEQ ID NO:148, SEQ ID NO:150, SEQ ID NO:152, SEQ ID NO:156, SEQ ID NO:158, and SEQ ID NO:160, respectively;
SEQ ID NO:164, SEQ ID NO:166, SEQ ID NO: 168, SEQ ID NO:172, SEQ ID NO:174, and SEQ ID NO:176, respectively; or
SEQ ID NO:180, SEQ ID NO:182, SEQ ID NO: 184, SEQ ID NO:188, SEQ ID NO:190, and SEQ ID NO:192, respectively.

9. An isolated antibody or antigen-binding protein that specifically binds to common gamma chain (γc) or an antigenic fragment thereof, wherein the isolated antibody or antigen-binding protein comprises:
(a) a heavy chain variable region that comprises the amino acid sequence set forth in SEQ ID NO: 2 and a light chain variable region that comprises the amino acid sequence set forth in SEQ ID NO: 10;
(b) a heavy chain variable region that comprises the amino acid sequence set forth in SEQ ID NO: 18 and a light chain variable region that comprises the amino acid sequence set forth in SEQ ID NO: 26;
(c) a heavy chain variable region that comprises the amino acid sequence set forth in SEQ ID NO: 34 and a light chain variable region that comprises the amino acid sequence set forth in SEQ ID NO: 42;
(d) a heavy chain variable region that comprises the amino acid sequence set forth in SEQ ID NO: 50 and a light chain variable region that comprises the amino acid sequence set forth in SEQ ID NO: 58;
(e) a heavy chain variable region that comprises the amino acid sequence set forth in SEQ ID NO: 66 and a light chain variable region that comprises the amino acid sequence set forth in SEQ ID NO: 74;
(f) a heavy chain variable region that comprises the amino acid sequence set forth in SEQ ID NO: 82 and a light chain variable region that comprises the amino acid sequence set forth in SEQ ID NO: 90;
(g) a heavy chain variable region that comprises the amino acid sequence set forth in SEQ ID NO: 98 and a light chain variable region that comprises the amino acid sequence set forth in SEQ ID NO: 106;
(h) a heavy chain variable region that comprises the amino acid sequence set forth in SEQ ID NO: 114 and a light chain variable region that comprises the amino acid sequence set forth in SEQ ID NO: 122;
(i) a heavy chain variable region that comprises the amino acid sequence set forth in SEQ ID NO: 130 and a light chain variable region that comprises the amino acid sequence set forth in SEQ ID NO: 138;
(j) a heavy chain variable region that comprises the amino acid sequence set forth in SEQ ID NO: 146 and a light chain variable region that comprises the amino acid sequence set forth in SEQ ID NO: 154;
(k) a heavy chain variable region that comprises the amino acid sequence set forth in SEQ ID NO: 162 and a light chain variable region that comprises the amino acid sequence set forth in SEQ ID NO: 170; or
(l) a heavy chain variable region that comprises the amino acid sequence set forth in SEQ ID NO: 178 and a light chain variable region that comprises the amino acid sequence set forth in SEQ ID NO: 184.

10. The isolated antibody or antigen-binding protein of claim 9, wherein said isolated antibody or antigen-binding protein is either a chimeric or humanized antibody constructed through framework patching method.

11. An isolated humanized antibody or antigen-binding protein that specifically binds to common gamma chain (γc) or an antigenic fragment thereof, wherein the isolated humanized antibody or antigen-binding protein comprises:
(a) a heavy chain variable region that comprises the amino acid sequence set forth in SEQ ID NO: 193 and a light chain variable region that comprises the amino acid sequence set forth in SEQ ID NO: 195;
(b) a heavy chain variable region that comprises the amino acid sequence set forth in SEQ ID NO: 197 and a light chain variable region that comprises the amino acid sequence set forth in SEQ ID NO: 199;
(c) a heavy chain variable region that comprises the amino acid sequence set forth in SEQ ID NO: 194 and a light chain variable region that comprises the amino acid sequence set forth in SEQ ID NO: 196; or
(d) a heavy chain variable region that comprises the amino acid sequence set forth in SEQ ID NO: 198 and a light chain variable region that comprises the amino acid sequence set forth in SEQ ID NO: 200.

12. The isolated antibody or antigen-binding protein of claim 9, wherein the isolated antibody or antigen-binding protein comprises a heavy chain comprising one of the heavy chain variable regions conjugated to a heavy chain constant region, wherein the amino acid sequence of said heavy chain constant region is set forth in SEQ ID NO: 201 or 203.

13. The isolated antibody or antigen-binding protein of claim 9, wherein the isolated antibody or antigen-binding protein comprises a light chain immunoglobulin comprising one of the light chain variable regions conjugated to a light chain constant region, wherein the amino acid sequence of said light chain constant region is set forth in SEQ ID NO: 202 or 204.

14.-17. (canceled)

18. A polynucleotide encoding one or more polypeptides of the isolated antigen-binding protein of claim 1.

19. A vector comprising the polynucleotide of claim 18.

20. A method of treating a common gamma chain (γc) or γc cytokine mediated autoimmune disease or condition selected from graft versus host disease (GVHD), organ transplant rejection, birdshot chorioretinopathy, multiple sclerosis (MS), uveitis, type 1 diabetes (T1D), atopic dermatitis (AD), rheumatoid arthritis (RA), systemic lupus erythematosus (SLE), asthma, psoriasis, Sjogren's syndrome (SS), vitiligo, celiac disease, Inflammatory bowel disease (IBD), alopecia areata (AA), mast cell mediated diseases, T cell lymphoma, NK cell lymphoma and B cell lymphoma in a subject in need thereof comprising the step of administering by injection an effective amount of the isolated antigen-binding protein of claim 1.

21. (canceled)

22. The isolated antigen-binding protein of claim 1, wherein said isolated antigen-binding protein is either a chimeric or humanized antibody constructed through framework patching method.

23. The isolated humanized antibody or antigen-binding protein of claim 11, wherein the isolated humanized antibody or antigen-binding protein comprises a heavy chain comprising one of the heavy chain variable regions conjugated to a heavy chain constant region, wherein the amino acid sequence of said heavy chain constant region is set forth in SEQ ID NO: 201 or 203.

24. The isolated humanized antibody or antigen-binding protein of claim 11, wherein the isolated humanized antibody or antigen-binding protein comprises a light chain comprising one of the light chain variable regions conjugated to a light chain constant region, wherein the amino acid sequence of said light chain constant region is set forth in SEQ ID NO: 202 or 204.

25. A method of treating a common gamma chain (γc) or γc cytokine mediated autoimmune disease or condition selected from among graft versus host disease (GVHD), organ transplant rejection, birdshot chorioretinopathy, multiple sclerosis (MS), uveitis, type 1 diabetes (T1D), atopic dermatitis (AD), rheumatoid arthritis (RA), systemic lupus erythematosus (SLE), asthma, psoriasis, Sjogren's syndrome (SS), vitiligo, celiac disease, Inflammatory bowel disease (IBD), alopecia areata (AA), mast cell mediated diseases, T cell lymphoma, NK cell lymphoma and B cell lymphoma in a subject in need thereof comprising the step of administering by injection an effective amount of the isolated antibody or antigen-binding protein of claim 9.

26. A method of treating a common gamma chain (γc) or γc cytokine mediated autoimmune disease or condition selected from among graft versus host disease (GVHD), organ transplant rejection, birdshot chorioretinopathy, multiple sclerosis (MS), uveitis, type 1 diabetes (T1D), atopic dermatitis (AD), rheumatoid arthritis (RA), systemic lupus erythematosus (SLE), asthma, psoriasis, Sjogren's syndrome (SS), vitiligo, celiac disease, Inflammatory bowel disease (IBD), alopecia areata (AA), mast cell mediated diseases, T cell lymphoma, NK cell lymphoma and B cell lymphoma in a subject in need thereof comprising the step of administering by injection an effective amount of the isolated humanized antibody or antigen-binding protein of claim 11.


SEQUENCE LISTING (237 sequences)

SEQ ID NO: 1 - DNA, 360 bp, synthetic construct
  source 1..360: mol_type = other DNA
gaggttcagcttcagcagtctggacctgaactggtgaagcctggggcttcagtgaagatatcctgcaagacttctggatacacattcactgaatacaccatgcactgggtgaagcagagccatggaaagagccttgagtggattggaggtattgatccttacaatggtggtgctacctacaaccagaggttcaagggcaaggccacattgactgtagacaagtcctccagcacagcctacatggagttccgcagcctgacatctgaggattctgcagtctattactgtgcaagaagaagactctactatggtaatgctctggactactggggtcaaggaacctcagtcaccgtctcctca

SEQ ID NO: 2 - protein, 120 aa, synthetic construct
EVQLQQSGPELVKPGASVKISCKTSGYTFTEYTMHWVKQSHGKSLEWIGGIDPYNGGATYNQRFKGKATLTVDKSSSTAYMEFRSLTSEDSAVYYCARRRLYYGNALDYWGQGTSVTVSS

SEQ ID NO: 3 - DNA, 15 bp, synthetic construct
  source 1..15: mol_type = other DNA
gaatacaccatgcac

SEQ ID NO: 4 - protein, 5 aa, synthetic construct
EYTMH

SEQ ID NO: 5 - DNA, 51 bp, synthetic construct
  source 1..51: mol_type = other DNA
ggtattgatccttacaatggtggtgctacctacaaccagaggttcaagggc

SEQ ID NO: 6 - protein, 17 aa, synthetic construct
GIDPYNGGATYNQRFKG

SEQ ID NO: 7 - DNA, 33 bp, synthetic construct
  source 1..33: mol_type = other DNA
agaagactctactatggtaatgctctggactac

SEQ ID NO: 8 - protein, 11 aa, synthetic construct
RRLYYGNALDY

SEQ ID NO: 9 - DNA, 339 bp, synthetic construct
  source 1..339: mol_type = other DNA
gatgttttgatgacccaaactccactctccctgcctgtcagtcttggagatcaagcctccatctcttgcagatctagtcagagccttgtacacagtaatggaaacacctatttacattggtacctgcagaggccaggccagtctccaaagctcctgatctacaaagtttccagccgattttctggggtcccagacaggttcagtggcagtggatcagggacagatttcacactcaagatcagcagagtggaggctgaggatctgggagtttatttctgctctcaaactacacatgttccattcacgttcggctcggggacaaagttggaaataaaacgg

SEQ ID NO: 10 - protein, 113 aa, synthetic construct
DVLMTQTPLSLPVSLGDQASISCRSSQSLVHSNGNTYLHWYLQRPGQSPKLLIYKVSSRFSGVPDRFSGSGSGTDFTLKISRVEAEDLGVYFCSQTTHVPFTFGSGTKLEIKR

SEQ ID NO: 11 - DNA, 48 bp, synthetic construct
  source 1..48: mol_type = other DNA
agatctagtcagagccttgtacacagtaatggaaacacctatttacat

SEQ ID NO: 12 - protein, 16 aa, synthetic construct
RSSQSLVHSNGNTYLH

SEQ ID NO: 13 - DNA, 21 bp, synthetic construct
  source 1..21: mol_type = other DNA
aaagtttccagccgattttct

SEQ ID NO: 14 - protein, 7 aa, synthetic construct
KVSSRFS

SEQ ID NO: 15 - DNA, 27 bp, synthetic construct
  source 1..27: mol_type = other DNA
tctcaaactacacatgttccattcacg

SEQ ID NO: 16 - protein, 9 aa, synthetic construct
SQTTHVPFT

SEQ ID NO: 17 - DNA, 360 bp, synthetic construct
  source 1..360: mol_type = other DNA
caggttcagctgcagcagtctggacctgaactggtgaagcctggggcttcagtgaagatattctgcaagacttctggatacacattcactgaatacaccatgcactgggtgaaacagagccatggagagagccttgagtggattggaggtattgatcctaacactggtggtgctacctacaaccagaagttcaagggcaaggccacattgactgcagacaagtcctccagcacagcctacatggagctccgtagcctgacatctgaggattctgcagtctattactgtgcaagaagaagacgctactatggttatgctttggactactggggtcaaggaacctcagtcaccgtctcctca

SEQ ID NO: 18 - protein, 120 aa, synthetic construct
QVQLQQSGPELVKPGASVKIFCKTSGYTFTEYTMHWVKQSHGESLEWIGGIDPNTGGATYNQKFKGKATLTADKSSSTAYMELRSLTSEDSAVYYCARRRRYYGYALDYWGQGTSVTVSS

SEQ ID NO: 19 - DNA, 15 bp, synthetic construct
  source 1..15: mol_type = other DNA
gaatacaccatgcac

SEQ ID NO: 20 - protein, 5 aa, synthetic construct
EYTMH

SEQ ID NO: 21 - DNA, 51 bp, synthetic construct
  source 1..51: mol_type = other DNA
ggtattgatcctaacactggtggtgctacctacaaccagaagttcaagggc

SEQ ID NO: 22 - protein, 17 aa, synthetic construct
GIDPNTGGATYNQKFKG

SEQ ID NO: 23 - DNA, 33 bp, synthetic construct
  source 1..33: mol_type = other DNA
agaagacgctactatggttatgctttggactac

SEQ ID NO: 24 - protein, 11 aa, synthetic construct
RRRYYGYALDY

SEQ ID NO: 25 - DNA, 339 bp, synthetic construct
  source 1..339: mol_type = other DNA
gatgttttgatgacccaaactccactctccctgcctgtcagtcttggagatcaagcctccatctcttgcagatctagtcagagcattgtacatagtaatggaaacacctatttagaatggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtggacgttcggtggaggcaccaagctggaaatcaaacgg

SEQ ID NO: 26 - protein, 113 aa, synthetic construct
DVLMTQTPLSLPVSLGDQASISCRSSQSIVHSNGNTYLEWYLQKPGQSPKLLIYKVSNRFSGVPDRFSGSGSGTDFTLKISRVEAEDLGVYFCSQSTHVPWTFGGGTKLEIKR

SEQ ID NO: 27 - DNA, 48 bp, synthetic construct
  source 1..48: mol_type = other DNA
agatctagtcagagcattgtacatagtaatggaaacacctatttagaa

SEQ ID NO: 28 - protein, 16 aa, synthetic construct
RSSQSIVHSNGNTYLE

SEQ ID NO: 29 - DNA, 21 bp, synthetic construct
  source 1..21: mol_type = other DNA
aaagtttccaaccgattttct

SEQ ID NO: 30 - protein, 7 aa, synthetic construct
KVSNRFS

SEQ ID NO: 31 - DNA, 27 bp, synthetic construct
  source 1..27: mol_type = other DNA
tctcaaagtacacatgttccgtggacg

SEQ ID NO: 32 - protein, 9 aa, synthetic construct
SQSTHVPWT

SEQ ID NO: 33 - DNA, 360 bp, synthetic construct
  source 1..360: mol_type = other DNA
caggtccaactgcagcaatctggacctgagctggtgaagcctggggcttcagtgaagatattctgcaagacttctggatacacattcactgaatacaccatgcactgggtgaaacagagccatggagagagccttgagtggattggaggtattgatcctaacactggtggtgctacctacaaccagaagttcaagggcaaggccacattgactgcagacaagtcctccagcacagcctacatggaactccgtagcctgacatctgaggattctgcagtctattactgtgcaagaagaagacgctactatggttatgctatggactactggggtcaaggaacctcagtcaccgtctcctca

SEQ ID NO: 34 - protein, 120 aa, synthetic construct
QVQLQQSGPELVKPGASVKIFCKTSGYTFTEYTMHWVKQSHGESLEWIGGIDPNTGGATYNQKFKGKATLTADKSSSTAYMELRSLTSEDSAVYYCARRRRYYGYAMDYWGQGTSVTVSS

SEQ ID NO: 35 - DNA, 15 bp, synthetic construct
  source 1..15: mol_type = other DNA
gaatacaccatgcac

SEQ ID NO: 36 - protein, 5 aa, synthetic construct
EYTMH

SEQ ID NO: 37 - DNA, 51 bp, synthetic construct; identical to SEQ ID NO: 21
  source 1..51: mol_type = other DNA

SEQ ID NO: 38 - protein, 17 aa, synthetic construct
GIDPNTGGATYNQKFKG

SEQ ID NO: 39 - DNA, 33 bp, synthetic construct
  source 1..33: mol_type = other DNA
agaagacgctactatggttatgctatggactac

SEQ ID NO: 40 - protein, 11 aa, synthetic construct
RRRYYGYAMDY

SEQ ID NO: 41 - DNA, 339 bp, synthetic construct
  source 1..339: mol_type = other DNA
gatgttgtgatgacccaaactccactctccctgcctgtcagtcttggagatcaagcctccatctcttgcagatctagtcagagccttgtacacagtaatggaaacacctatttacattggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggaatttattactgctttcaagattcacatgttccattcacgttcggctcggggacaaagttggaaataaaacgg

SEQ ID NO: 42 - protein, 113 aa, synthetic construct
DVVMTQTPLSLPVSLGDQASISCRSSQSLVHSNGNTYLHWYLQKPGQSPKLLIYKVSNRFSGVPDRFSGSGSGTDFTLKISRVEAEDLGIYYCFQDSHVPFTFGSGTKLEIKR

SEQ ID NO: 43 - DNA, 48 bp, synthetic construct; identical to SEQ ID NO: 11
  source 1..48: mol_type = other DNA

SEQ ID NO: 44 - protein, 16 aa, synthetic construct
RSSQSLVHSNGNTYLH

SEQ ID NO: 45 - DNA, 21 bp, synthetic construct
  source 1..21: mol_type = other DNA
aaagtttccaaccgattttct

SEQ ID NO: 46 - protein, 7 aa, synthetic construct
KVSNRFS

SEQ ID NO: 47 - DNA, 27 bp, synthetic construct
  source 1..27: mol_type = other DNA
tttcaagattcacatgttccattcacg

SEQ ID NO: 48 - protein, 9 aa, synthetic construct
FQDSHVPFT

SEQ ID NO: 49 - DNA, 360 bp, synthetic construct
  source 1..360: mol_type = other DNA
caggttcaactgcagcagtctggacctgaactggtgaagcctggggcttcagtgaagatatcctgcaagacttctggatacacattcactgaatacaccatgcactgggtgaagcagagccatggaaagagccttgagtggattggaggtattgatccttacaatggtggtgttacctacaaccagaggttcaagggcaaggccacattgactgtagacaagtcctccagcacagcctacatggagttccgcagcctgacatctgaggattctgcagtctattactgtgcaagaagaagactctattatggtaatgctctggactactggggtcaaggaacctcagtcaccgtctcctca

SEQ ID NO: 50 - protein, 120 aa, synthetic construct
QVQLQQSGPELVKPGASVKISCKTSGYTFTEYTMHWVKQSHGKSLEWIGGIDPYNGGVTYNQRFKGKATLTVDKSSSTAYMEFRSLTSEDSAVYYCARRRLYYGNALDYWGQGTSVTVSS

SEQ ID NO: 51 - DNA, 15 bp, synthetic construct
  source 1..15: mol_type = other DNA
gaatacaccatgcac

SEQ ID NO: 52 - protein, 5 aa, synthetic construct
EYTMH

SEQ ID NO: 53 - DNA, 51 bp, synthetic construct
  source 1..51: mol_type = other DNA
ggtattgatccttacaatggtggtgttacctacaaccagaggttcaagggc

SEQ ID NO: 54 - protein, 17 aa, synthetic construct
GIDPYNGGVTYNQRFKG

SEQ ID NO: 55 - DNA, 33 bp, synthetic construct
  source 1..33: mol_type = other DNA
agaagactctattatggtaatgctctggactac

SEQ ID NO: 56 - protein, 11 aa, synthetic construct
RRLYYGNALDY

SEQ ID NO: 57 - DNA, 339 bp, synthetic construct
  source 1..339: mol_type = other DNA
gatattgtgatgacccaaactccactctccctgcctgtcagtcttggagatcaagcctccatctcttgcagatctagtcagagcattgtacataggaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccattcacgttcggctcggggacaaagttggaaataaaacgg

SEQ ID NO: 58 - protein, 113 aa, synthetic construct
DIVMTQTPLSLPVSLGDQASISCRSSQSIVHRNGNTYLEWYLQKPGQSPKLLIYKVSNRFSGVPDRFSGSGSGTDFTLKISRVEAEDLGVYYCFQGSHVPFTFGSGTKLEIKR

SEQ ID NO: 59 - DNA, 48 bp, synthetic construct
  source 1..48: mol_type = other DNA
agatctagtcagagcattgtacataggaatggaaacacctatttagaa

SEQ ID NO: 60 - protein, 16 aa, synthetic construct
RSSQSIVHRNGNTYLE

SEQ ID NO: 61 - DNA, 21 bp, synthetic construct
  source 1..21: mol_type = other DNA
aaagtttccaaccgattttct

SEQ ID NO: 62 - protein, 7 aa, synthetic construct
KVSNRFS

SEQ ID NO: 63 - DNA, 27 bp, synthetic construct
  source 1..27: mol_type = other DNA
tttcaaggttcacatgttccattcacg

SEQ ID NO: 64 - protein, 9 aa, synthetic construct
FQGSHVPFT

SEQ ID NO: 65 - DNA, 360 bp, synthetic construct
  source 1..360: mol_type = other DNA
gaggtccatctgcagcaatctggacctgaactggtgaagcctggggcttcagtgaagatattctgcaagacttctggatacacattcactgaatacaccatgcactgggtgaaacagagccatggagagagccttgagtggattggaggtattgatcctaacactggtggtgttacctacaaccagaagttcaagggcaaggccacattgactgcagacaagtcctccagcacagcctacatggagctccgtagcctgacatctgaggattctgcagtctattactgtgcaagaagaagacgctactatggttatgctttggactactggggtcaaggaacctcagtcaccgtctcctca

SEQ ID NO: 66 - protein, 120 aa, synthetic construct
EVHLQQSGPELVKPGASVKIFCKTSGYTFTEYTMHWVKQSHGESLEWIGGIDPNTGGVTYNQKFKGKATLTADKSSSTAYMELRSLTSEDSAVYYCARRRRYYGYALDYWGQGTSVTVSS

SEQ ID NO: 67 - DNA, 15 bp, synthetic construct
  source 1..15: mol_type = other DNA
gaatacaccatgcac

SEQ ID NO: 68 - protein, 5 aa, synthetic construct
EYTMH

SEQ ID NO: 69 - DNA, 51 bp, synthetic construct
  source 1..51: mol_type = other DNA
ggtattgatcctaacactggtggtgttacctacaaccagaagttcaagggc

SEQ ID NO: 70 - protein, 17 aa, synthetic construct
GIDPNTGGVTYNQKFKG

SEQ ID NO: 71 - DNA, 33 bp, synthetic construct; identical to SEQ ID NO: 23
  source 1..33: mol_type = other DNA

SEQ ID NO: 72 - protein, 11 aa, synthetic construct
RRRYYGYALDY

SEQ ID NO: 73 - DNA, 339 bp, synthetic construct
  source 1..339: mol_type = other DNA
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagcgtggaggctgaggatctgggagtttatttctgctctcaaagtacacatgttccattcatgttcggctcggggacaaagttggaaataaaacgg

SEQ ID NO: 74 - protein, 113 aa, synthetic construct
DVLMTQTPLSLPVSLGDQASISCRSSQSIVHSNGNTYLEWYLQKPGQSPKLLIYKVSNRFSGVPDRFSGSGSGTDFTLKISSVEAEDLGVYFCSQSTHVPFMFGSGTKLEIKR

SEQ ID NO: 75 - DNA, 48 bp, synthetic construct; identical to SEQ ID NO: 27
  source 1..48: mol_type = other DNA

SEQ ID NO: 76 - protein, 16 aa, synthetic construct
RSSQSIVHSNGNTYLE

SEQ ID NO: 77 - DNA, 21 bp, synthetic construct
  source 1..21: mol_type = other DNA
aaagtttccaaccgattttct

SEQ ID NO: 78 - protein, 7 aa, synthetic construct
KVSNRFS

SEQ ID NO: 79 - DNA, 27 bp, synthetic construct
  source 1..27: mol_type = other DNA
tctcaaagtacacatgttccattcatg

SEQ ID NO: 80 - protein, 9 aa, synthetic construct
SQSTHVPFM

SEQ ID NO: 81 - DNA, 360 bp, synthetic construct
  source 1..360: mol_type = other DNA
gaggttcagcttcagcagtctggacctgaactggtgaagcctggggcttcagtgaagatattctgcaagacttctggatacacattcactgaatacaccatgcactgggtgaaacagagccatggagagagccttgagtggattggaggtattgatcctaacactggtggtgttacctacaaccagaagttcaagggcaaggccacattgactgcagacaagtcctccagcacagcctacatggagctccgtagcctgacatctgaggattctgcagtctattactgtgcaagaagaagacgctactatggttatgctttggactactggggtcaaggaacctcagtcaccgtctcctca

SEQ ID NO: 82 - protein, 120 aa, synthetic construct
EVQLQQSGPELVKPGASVKIFCKTSGYTFTEYTMHWVKQSHGESLEWIGGIDPNTGGVTYNQKFKGKATLTADKSSSTAYMELRSLTSEDSAVYYCARRRRYYGYALDYWGQGTSVTVSS

SEQ ID NO: 83 - DNA, 15 bp, synthetic construct
  source 1..15: mol_type = other DNA
gaatacaccatgcac

SEQ ID NO: 84 - protein, 5 aa, synthetic construct
EYTMH

SEQ ID NO: 85 - DNA, 51 bp, synthetic construct; identical to SEQ ID NO: 69
  source 1..51: mol_type = other DNA

SEQ ID NO: 86 - protein, 17 aa, synthetic construct
GIDPNTGGVTYNQKFKG

SEQ ID NO: 87 - DNA, 33 bp, synthetic construct; identical to SEQ ID NO: 23
  source 1..33: mol_type = other DNA

SEQ ID NO: 88 - protein, 11 aa, synthetic construct
RRRYYGYALDY

SEQ ID NO: 89 - DNA, 339 bp, synthetic construct
  source 1..339: mol_type = other DNA
gatgttttgatgacccaaactccactctccctgcctgtcagtcttggagatcaagcctccatctcttgcagatctagtcagagcattgtacataataatggaaacacctatttagaatggtacctgcagaaaccaggccagtctccaaagctcctgatctacaaagtttccaaccgattttttggggtcccagacaggttcagtggcagtggatcagggacttatttcacactcaagatcagcagagtggaggctgaggatctgggagtttattactgctttcaaggttcacatgttccgtggacgttcggtggaggcaccaagctggaaatcaaacgg

SEQ ID NO: 90 - protein, 113 aa, synthetic construct
DVLMTQTPLSLPVSLGDQASISCRSSQSIVHNNGNTYLEWYLQKPGQSPKLLIYKVSNRFFGVPDRFSGSGSGTYFTLKISRVEAEDLGVYYCFQGSHVPWTFGGGTKLEIKR

SEQ ID NO: 91 - DNA, 48 bp, synthetic construct
  source 1..48: mol_type = other DNA
agatctagtcagagcattgtacataataatggaaacacctatttagaa

SEQ ID NO: 92 - protein, 16 aa, synthetic construct
RSSQSIVHNNGNTYLE

SEQ ID NO: 93 - DNA, 21 bp, synthetic construct
  source 1..21: mol_type = other DNA
aaagtttccaaccgatttttt

SEQ ID NO: 94 - protein, 7 aa, synthetic construct
KVSNRFF

SEQ ID NO: 95 - DNA, 27 bp, synthetic construct
  source 1..27: mol_type = other DNA
tttcaaggttcacatgttccgtggacg

SEQ ID NO: 96 - protein, 9 aa, synthetic construct
FQGSHVPWT

SEQ ID NO: 97 - DNA, 360 bp, synthetic construct
  source 1..360: mol_type = other DNA
gaggttcagcttcagcagtctggacctgagctggtgaagcctggggcttcagtgaagatattctgcaagacttctggatacacattcactgaatacaccatgcactgggtgaaacagagccatggagagagccttgagtggattggaggtattgatcctaacactggtggtgttacctacaaccagaagttcaagggcaaggccacattgactgcagacagttcctccagcacagcctacatggagctccgtaacctgacatctgaggattctgcagtctattactgtgcaagaagaagacgctactatggttatgctttggactactggggtcaaggaacctcagtcaccgtctcctca

SEQ ID NO: 98 - protein, 120 aa, synthetic construct
EVQLQQSGPELVKPGASVKIFCKTSGYTFTEYTMHWVKQSHGESLEWIGGIDPNTGGVTYNQKFKGKATLTADSSSSTAYMELRNLTSEDSAVYYCARRRRYYGYALDYWGQGTSVTVSS

SEQ ID NO: 99 - DNA, 15 bp, synthetic construct
  source 1..15: mol_type = other DNA
gaatacaccatgcac

SEQ ID NO: 100 - protein, 5 aa, synthetic construct
EYTMH

SEQ ID NO: 101 - DNA, 51 bp, synthetic construct; identical to SEQ ID NO: 69
  source 1..51: mol_type = other DNA

SEQ ID NO: 102 - protein, 17 aa, synthetic construct
GIDPNTGGVTYNQKFKG

SEQ ID NO: 103 - DNA, 33 bp, synthetic construct; identical to SEQ ID NO: 23
  source 1..33: mol_type = other DNA

SEQ ID NO: 104 - protein, 11 aa, synthetic construct
RRRYYGYALDY

SEQ ID NO: 105 - DNA, 339 bp, synthetic construct
  source 1..339: mol_type = other DNA
gatgttgtgatgacccaaactccactctccctgcctgtcaatcttggagatcaagcctccatctcttgcagatctagtcagagccttgtacatagtaatggaaacacctatttagattggtacctgcagaaaccaggccagtctccaaagctcctgatctacaaagtttccaaccgattttctggggtcccagacaggttcagtggcagtgggtcagggacatatttcacactcaatttcagcagagtggaggctgaggatctgggagtttattactgctttcaaggttcacatgttccattcacgttcggctcggggacaaagttggaaataaaacgg

SEQ ID NO: 106 - protein, 113 aa, synthetic construct
DVVMTQTPLSLPVNLGDQASISCRSSQSLVHSNGNTYLDWYLQKPGQSPKLLIYKVSNRFSGVPDRFSGSGSGTYFTLNFSRVEAEDLGVYYCFQGSHVPFTFGSGTKLEIKR

SEQ ID NO: 107 - DNA, 48 bp, synthetic construct
  source 1..48: mol_type = other DNA
agatctagtcagagccttgtacatagtaatggaaacacctatttagat

SEQ ID NO: 108 - protein, 16 aa, synthetic construct
RSSQSLVHSNGNTYLD

SEQ ID NO: 109 - DNA, 21 bp, synthetic construct
  source 1..21: mol_type = other DNA
aaagtttccaaccgattttct

SEQ ID NO: 110 - protein, 7 aa, synthetic construct
KVSNRFS

SEQ ID NO: 111 - DNA, 27 bp, synthetic construct
  source 1..27: mol_type = other DNA
tttcaaggttcacatgttccattcacg

SEQ ID NO: 112 - protein, 9 aa, synthetic construct
FQGSHVPFT

SEQ ID NO: 113 - DNA, 360 bp, synthetic construct
  source 1..360: mol_type = other DNA
caggttcaactgcagcagtctggacctgagctggtgaagcctggggcttcaatgaacatattctgcaagacttctggatacacattcactgaatacaccatgcactgggtgaaacagagccatggagagagccttgagtggattggaggtattgatcctaacactggtggtgctacctacaaccagaagttcaagggccaggccacattgactgcagacaagtcctccagcacagcctacatggagctccgtagcctgacatctgaagattctgcagtctattactgtgcaagaagaagacgctactatggttatgctttggactactggggtcaaggaacctcagtcaccgtctcctca

SEQ ID NO: 114 - protein, 120 aa, synthetic construct
QVQLQQSGPELVKPGASMNIFCKTSGYTFTEYTMHWVKQSHGESLEWIGGIDPNTGGATYNQKFKGQATLTADKSSSTAYMELRSLTSEDSAVYYCARRRRYYGYALDYWGQGTSVTVSS

SEQ ID NO: 115 - DNA, 15 bp, synthetic construct
  source 1..15: mol_type = other DNA
gaatacaccatgcac

SEQ ID NO: 116 - protein, 5 aa, synthetic construct
EYTMH

SEQ ID NO: 117 - DNA, 51 bp, synthetic construct; identical to SEQ ID NO: 21
  source 1..51: mol_type = other DNA

SEQ ID NO: 118 - protein, 17 aa, synthetic construct
GIDPNTGGATYNQKFKG

SEQ ID NO: 119 - DNA, 33 bp, synthetic construct; identical to SEQ ID NO: 23
  source 1..33: mol_type = other DNA

SEQ ID NO: 120 - protein, 11 aa, synthetic construct
RRRYYGYALDY

SEQ ID NO: 121 - DNA, 339 bp, synthetic construct
  source 1..339: mol_type = other DNA
gatgttttgatgacccaaactccactctccctgcctgtcagtcttggagatcaagcctccatctcttgcagatctagtcagaacattatacatagtaatggaaacacctatttagaatggtacctgcagaaaccaggccagtctccaaagctcctgatctacaaagtttccaaccgattttctggggtccctgacaggttcactggcagtgggtcagggacagatttcacactcaagatcagcagagtggaggctgaggatctgggagtttatttctgctctcaaagtacacatgttccattcacgttcggtggaggcaccaagctggaaatcaaacgg

SEQ ID NO: 122 - protein, 113 aa, synthetic construct
DVLMTQTPLSLPVSLGDQASISCRSSQNIIHSNGNTYLEWYLQKPGQSPKLLIYKVSNRFSGVPDRFTGSGSGTDFTLKISRVEAEDLGVYFCSQSTHVPFTFGGGTKLEIKR

SEQ ID NO: 123 - DNA, 48 bp, synthetic construct
  source 1..48: mol_type = other DNA
agatctagtcagaacattatacatagtaatggaaacacctatttagaa

SEQ ID NO: 124 - protein, 16 aa, synthetic construct
RSSQNIIHSNGNTYLE

SEQ ID NO: 125 - DNA, 21 bp, synthetic construct
  source 1..21: mol_type = other DNA
aaagtttccaaccgattttct

SEQ ID NO: 126 - protein, 7 aa, synthetic construct
KVSNRFS

SEQ ID NO: 127 - DNA, 27 bp, synthetic construct
  source 1..27: mol_type = other DNA
tctcaaagtacacatgttccattcacg

SEQ ID NO: 128 - protein, 9 aa, synthetic construct
SQSTHVPFT

SEQ ID NO: 129 - DNA, 360 bp, synthetic construct
  source 1..360: mol_type = other DNA
caggttcaactgcagcagtctggggctgaactggcaaaacctggggcttcagtgaagctgtcctgcaaggcttctggatacacattcactgaatacaccatgcactgggtgaaacagagccatggagagagccttgactggattggaggtattgatcctaacactggtggtgctacctacaaccagaggttcaagggcaaggccacaatgactgtagataagtcctccagtacagcctacatggagttccgaagcctgacatctgaggattctgcagtctattactgtgcaagaagaagacgctactatggttatgctttggacaactggggtcaaggaacctcagtcaccgtctcctca

SEQ ID NO: 130 - protein, 120 aa, synthetic construct
QVQLQQSGAELAKPGASVKLSCKASGYTFTEYTMHWVKQSHGESLDWIGGIDPNTGGATYNQRFKGKATMTVDKSSSTAYMEFRSLTSEDSAVYYCARRRRYYGYALDNWGQGTSVTVSS

SEQ ID NO: 131 - DNA, 15 bp, synthetic construct
  source 1..15: mol_type = other DNA
gaatacaccatgcac

SEQ ID NO: 132 - protein, 5 aa, synthetic construct
EYTMH

SEQ ID NO: 133 - DNA, 51 bp, synthetic construct
  source 1..51: mol_type = other DNA
ggtattgatcctaacactggtggtgctacctacaaccagaggttcaagggc

SEQ ID NO: 134 - protein, 17 aa, synthetic construct
GIDPNTGGATYNQRFKG

SEQ ID NO: 135 - DNA, 33 bp, synthetic construct
  source 1..33: mol_type = other DNA
agaagacgctactatggttatgctttggacaac

SEQ ID NO: 136 - protein, 11 aa, synthetic construct
RRRYYGYALDN

SEQ ID NO: 137 - DNA, 339 bp, synthetic construct
  source 1..339: mol_type = other DNA
gatgttttgatgacccaaactccactctccctgcctgtcagtcttggagatcaagcctccatctcttgcagatctagtcagagcattgtccatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaagattcacatgttccattcacgttcggctcggggacaaagttggaaataaaacgg

SEQ ID NO: 138 - protein, 113 aa, synthetic construct
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQDSHVPFTFGSGTKLEIKR

SEQ ID NO: 139 - DNA, 48 bp, synthetic construct
  source 1..48: mol_type = other DNA
agatctagtcagagcattgtccatagtaatggaaacacctatttagaa

SEQ ID NO: 140 - protein, 16 aa, synthetic construct
RSSQSIVHSNGNTYLE

SEQ ID NO: 141 - DNA, 21 bp, synthetic construct
  source 1..21: mol_type = other DNA
aaagtttccaaccgattttct

SEQ ID NO: 142 - protein, 7 aa, synthetic construct
KVSNRFS

SEQ ID NO: 143 - DNA, 27 bp, synthetic construct
  source 1..27: mol_type = other DNA
tttcaagattcacatgttccattcacg

SEQ ID NO: 144 - protein, 9 aa, synthetic construct
FQDSHVPFT

SEQ ID NO: 145 - DNA, 360 bp, synthetic construct
  source 1..360: mol_type = other DNA
gaggttcagctgcaacagtctggacctgaactggtgaagcctggggcttcagtgaagatatcctgcaagacttctggatacacattcactgaatacaccatgcactgggtgaagcagagccatggaaagagccttgagtggattggaggtattgatccttacaatggtggtattacctacaaccagaggttcaagggcaaggccacattgactgtagacaagtcctccagcacagcctacatggagctccgcagcctgacatctgaggattctgcagtctattactgtgcaagaagaagactctactatggttatgctctggactactggggtcaaggaacctcagtcaccgtctcctca

SEQ ID NO: 146 - protein, 120 aa, synthetic construct
EVQLQQSGPELVKPGASVKISCKTSGYTFTEYTMHWVKQSHGKSLEWIGGIDPYNGGITYNQRFKGKATLTVDKSSSTAYMELRSLTSEDSAVYYCARRRLYYGYALDYWGQGTSVTVSS

SEQ ID NO: 147 - DNA, 15 bp, synthetic construct
  source 1..15: mol_type = other DNA
gaatacaccatgcac

SEQ ID NO: 148 - protein, 5 aa, synthetic construct
EYTMH

SEQ ID NO: 149 - DNA, 51 bp, synthetic construct
  source 1..51: mol_type = other DNA
ggtattgatccttacaatggtggtattacctacaaccagaggttcaagggc

SEQ ID NO: 150 - protein, 17 aa, synthetic construct
GIDPYNGGITYNQRFKG

SEQ ID NO: 151 - DNA, 33 bp, synthetic construct
  source 1..33: mol_type = other DNA
agaagactctactatggttatgctctggactac

SEQ ID NO: 152 - protein, 11 aa, synthetic construct
RRLYYGYALDY

SEQ ID NO: 153 - DNA, 339 bp, synthetic construct
  source 1..339: mol_type = other DNA
gatgttgtgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccattcacgttcggctcggggacaaagttggaaataaaacgg

SEQ ID NO: 154 - protein, 113 aa, synthetic construct
DVVMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPFTFGSGTKLEIKR

SEQ ID NO: 155 - DNA, 48 bp, synthetic construct; identical to SEQ ID NO: 27
  source 1..48: mol_type = other DNA

SEQ ID NO: 156 - protein, 16 aa, synthetic construct
RSSQSIVHSNGNTYLE

SEQ ID NO: 157 - DNA, 21 bp, synthetic construct
  source 1..21: mol_type = other DNA
aaagtttccaaccgattttct

SEQ ID NO: 158 - protein, 7 aa, synthetic construct
KVSNRFS

SEQ ID NO: 159 - DNA, 27 bp, synthetic construct
  source 1..27: mol_type = other DNA
tttcaaggttcacatgttccattcacg

SEQ ID NO: 160 - protein, 9 aa, synthetic construct
FQGSHVPFT

SEQ ID NO: 161 - DNA, 360 bp, synthetic construct
  source 1..360: mol_type = other DNA
gaagtgcagctgttggagtctggacctgaactggtgaagcctggggcttcagtgaagatatcctgcaaaacttctggttactcatttactgactactttatgaactgggtgaagcagagccatggagagagccttgagtggattggaggaattgatcctaatactggaggtgttacctacaaccagaagttcaagggcaaggccacattgactgtagacaagtccgccagtacagcctacatggagctccgtagcctgacatctgaggattctgcagtctattactgtgcaagaagaagacgctactatggttatgctatggactactggggtcaaggaacctcagtcaccgtctcctca

SEQ ID NO: 162 - protein, 120 aa, synthetic construct
EVQLLESGPELVKPGASVKISCKTSGYSFTDYFMNWVKQSHGESLEWIGGIDPNTGGVTYNQKFKGKATLTVDKSASTAYMELRSLTSEDSAVYYCARRRRYYGYAMDYWGQGTSVTVSS

SEQ ID NO: 163 - DNA, 15 bp, synthetic construct
  source 1..15: mol_type = other DNA
gactactttatgaac

SEQ ID NO: 164 - protein, 5 aa, synthetic construct
DYFMN

SEQ ID NO: 165 - DNA, 51 bp, synthetic construct
  source 1..51: mol_type = other DNA
ggaattgatcctaatactggaggtgttacctacaaccagaagttcaagggc

SEQ ID NO: 166 - protein, 17 aa, synthetic construct
GIDPNTGGVTYNQKFKG

SEQ ID NO: 167 - DNA, 33 bp, synthetic construct; identical to SEQ ID NO: 39
  source 1..33: mol_type = other DNA

SEQ ID NO: 168 - protein, 11 aa, synthetic construct
RRRYYGYAMDY

SEQ ID NO: 169 - DNA, 339 bp, synthetic construct
  source 1..339: mol_type = other DNA
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtggacgttcggtggaggcaccaagctggaaataaaacgg

SEQ ID NO: 170 - protein, 113 aa, synthetic construct; identical to SEQ ID NO: 26

SEQ ID NO: 171 - DNA, 48 bp, synthetic construct; identical to SEQ ID NO: 27
  source 1..48: mol_type = other DNA

SEQ ID NO: 172 - protein, 16 aa, synthetic construct
RSSQSIVHSNGNTYLE

SEQ ID NO: 173 - DNA, 21 bp, synthetic construct
  source 1..21: mol_type = other DNA
aaagtttccaaccgattttct

SEQ ID NO: 174 - protein, 7 aa, synthetic construct
KVSNRFS

SEQ ID NO: 175 - DNA, 27 bp, synthetic construct
  source 1..27: mol_type = other DNA
tctcaaagtacacatgttccgtggacg

SEQ ID NO: 176 - protein, 9 aa, synthetic construct
SQSTHVPWT

SEQ ID NO: 177 - DNA, 360 bp, synthetic construct
  source 1..360: mol_type = other DNA
gaggttcagctgcagcaatctggacctgagctggtgaagcctggggcttcagtgaagatatcctgcaagacttctggatacacattcactgaatacaccatgcactgggtgaagcagagccatggaaagagccttgagtggattggaggtattgatcctaacagtggtggtgctacctacaaccagaagttcaagggcaaggccacattgactgtagacaagtcctccagcacagcctacatggaggtccgcagcctgacatctgaggattctgcagtctattactgtgcaagaagaagactctactatggttatgctatggactactggggtcaaggaacctcagtcaccgtctcctca

SEQ ID NO: 178 - protein, 120 aa, synthetic construct
EVQLQQSGPELVKPGASVKISCKTSGYTFTEYTMHWVKQSHGKSLEWIGGIDPNSGGATYNQKFKGKATLTVDKSSSTAYMEVRSLTSEDSAVYYCARRRLYYGYAMDYWGQGTSVTVSS

SEQ ID NO: 179 - DNA, 15 bp, synthetic construct
  source 1..15: mol_type = other DNA
gaatacaccatgcac

SEQ ID NO: 180 - protein, 5 aa, synthetic construct
EYTMH

SEQ ID NO: 181 - DNA, 51 bp, synthetic construct
  source 1..51: mol_type = other DNA
ggtattgatcctaacagtggtggtgctacctacaaccagaagttcaagggc

SEQ ID NO: 182 - protein, 17 aa, synthetic construct
GIDPNSGGATYNQKFKG

SEQ ID NO: 183 - DNA, 33 bp, synthetic construct
  source 1..33: mol_type = other DNA
agaagactctactatggttatgctatggactac

SEQ ID NO: 184 - protein, 11 aa, synthetic construct
RRLYYGYAMDY

SEQ ID NO: 185 - DNA, 339 bp, synthetic construct
  source 1..339: mol_type = other DNA
gatgttttgatgacccaaactccactctccctgcctgtcagtcttggaggtcaagcctccatctcttgcagatctagtcagaacattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccattcgcgttcggctcggggacaaagttggaaataaaacgg

SEQ ID NO: 186 - protein, 113 aa, synthetic construct
DVLMTQTPLSLPVSLGGQASISCRSSQNIVHSNGNTYLEWYLQKPGQSPKLLIYKVSNRFSGVPDRFSGSGSGTDFTLKISRVEAEDLGVYFCSQSTHVPFAFGSGTKLEIKR

SEQ ID NO: 187 - DNA, 48 bp, synthetic construct
  source 1..48: mol_type = other DNA
agatctagtcagaacattgtacatagtaatggaaacacctatttagaa

SEQ ID NO: 188 - protein, 16 aa, synthetic construct
RSSQNIVHSNGNTYLE

SEQ ID NO: 189 - DNA, 21 bp, synthetic construct
  source 1..21: mol_type = other DNA
aaagtttccaaccgattttct

SEQ ID NO: 190 - protein, 7 aa, synthetic construct
KVSNRFS

SEQ ID NO: 191 - DNA, 27 bp, synthetic construct
  source 1..27: mol_type = other DNA
tctcaaagtacacatgttccattcgcg

SEQ ID NO: 192 - protein, 9 aa, synthetic construct
SQSTHVPFA

SEQ ID NO: 193 - protein, 120 aa, synthetic construct
QVQLVQSGAEVLKPGASVKVSCKASGYTFTEYTMHWVRQARGQRLEWIGGIDPYNGGATYNQRFKGRVTMTTDTSTSTAYMELRNLRSDDTAVYYCARRRLYYGNALDYWGQGTLVTVSS

SEQ ID NO: 194 - protein, 120 aa, synthetic construct
QVQLVQSGAEVLKPGASVKVSCKASGYTFTEYTMHWVRQARGQRLEWIGGIDPNTGGVTYNQKFKGRVTMTTDTSTSTAYMELRNLRSDDTAVYYCARRRRYYGYALDYWGQGTLVTVSS

SEQ ID NO: 195 - protein, 113 aa, synthetic construct
DIVMTQSPLSLPVTPGEPASISCRSSQSLVHSNGNTYLHWYLQKPGQSPQLLIYKVSSRFSGVPDRFSGSGSGTDFTLKISRVEAEDVGVYYCSQTTHVPFTFGQGTKLEIKR

SEQ ID NO: 196 - protein, 113 aa, synthetic construct
DIVMTQSPLSLPVTPGEPASISCRSSQSIVHSNGNTYLEWYLQKPGQSPQLLIYKVSNRFSGVPDRFSGSGSGTDFTLKISRVEAEDVGVYYCSQSTHVPFMFGQGTKLEIKR

SEQ ID NO: 197 - protein, 120 aa, synthetic construct
QVQLVQSGAEVLKPGASVKVSCKASGYTFTEYTMHWVKQARGQRLEWIGGIDPYNGGATYNQRFKGKATLTTDTSTSTAYMELRNLRSDDTAVYYCARRRLYYGNALDYWGQGTLVTVSS

SEQ ID NO: 198 - protein, 120 aa, synthetic construct
QVQLVQSGAEVLKPGASVKVSCKASGYTFTEYTMHWVKQARGQRLEWIGGIDPNTGGVTYNQKFKGKATLTTDTSTSTAYMELRNLRSDDTAVYYCARRRRYYGYALDYWGQGTLVTVSS

SEQ ID NO: 199 - protein, 113 aa, synthetic construct
DIVMTQSPLSLPVTPGEQASISCRSSQSLVHSNGNTYLHWYLQKPGQSPQLLIYKVSSRFSGVPDRFSGSGSGTDFTLKISRVEAEDVGVYFCSQTTHVPFTFGSGTKLEIKR

SEQ ID NO: 200 - protein, 113 aa, synthetic construct
DIVMTQSPLSLPVTPGEQASISCRSSQSIVHSNGNTYLEWYLQKPGQSPQLLIYKVSNRFSGVPDRFSGSGSGTDFTLKISRVEAEDVGVYFCSQSTHVPFMFGSGTKLEIKR

SEQ ID NO: 201 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 202 - protein, 106 aa, synthetic construct
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 203 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 204 - protein, 106 aa, synthetic construct
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 205 - protein, 509 aa, synthetic construct
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAGGGGSGGGGSGGGGSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKTISRSLGK

SEQ ID NO: 206 - protein, 268 aa, synthetic construct
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAHHHHHH

SEQ ID NO: 207 - protein, 501 aa, synthetic construct
MLKPSLPFRSLLVLQLPLLGVALNTTILTPNGNEDATTDFFLTSMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLRKLSESQLELNWNNRFLNHCLEHLVQYRTDWDHSWTEQSVDYRHKFSLPSVDGQKRYTFRVRSRFNPLCGSAQHWSEWSHPIHWGSNSSKENGGGGSGGGGSGGGGSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKTISRSLGK

SEQ ID NO: 208 - protein, 260 aa, synthetic construct
MLKPSLPFRSLLVLQLPLLGVALNTTILTPNGNEDATTDFFLTSMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLRKLSESQLELNWNNRFLNHCLEHLVQYRTDWDHSWTEQSVDYRHKFSLPSVDGQKRYTFRVRSRFNPLCGSAQHWSEWSHPIHWGSNSSKENHHHHHH

SEQ ID NO: 209 - protein, 509 aa, synthetic construct
MLKPSLPFTSLLFLQLPLLGVGLNTTILTPNGNEDTTADFFLTSMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAGGGGSGGGGSGGGGSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKTISRSLGK

SEQ ID NO: 210 - protein, 268 aa, synthetic construct
MLKPSLPFTSLLFLQLPLLGVGLNTTILTPNGNEDTTADFFLTSMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAHHHHHH

SEQ ID NO: 211 - protein, 508 aa, synthetic construct
MLKALLPFRSLLFLQLPLLGVGLNTILTPNGNEDTTADFFLTSMPTDSFSVSTLPLPEVQCFVFNVEYMNCTWNSSSEPQPTNLTLHYWYKNSDNDKVQKCSHYLFAEEITSGCQLQKKEIHLYQTFVVQLQDPREPRRQATQMLKLQNLVIPWAPENLTLRKLSESQLELNWNNRFLNHCLEHLVQYRTDWDHTWTEQSVGHSHSFSLPSVDGQKHYTFRVRSRFNPLCGSTQHWSEWSHLIHWGSNTSKENPFLFALEAGGGGSGGGGSGGGGSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKTISRSLGK

SEQ ID NO: 212 - protein, 267 aa, synthetic construct
MLKALLPFRSLLFLQLPLLGVGLNTILTPNGNEDTTADFFLTSMPTDSFSVSTLPLPEVQCFVFNVEYMNCTWNSSSEPQPTNLTLHYWYKNSDNDKVQKCSHYLFAEEITSGCQLQKKEIHLYQTFVVQLQDPREPRRQATQMLKLQNLVIPWAPENLTLRKLSESQLELNWNNRFLNHCLEHLVQYRTDWDHTWTEQSVGHSHSFSLPSVDGQKHYTFRVRSRFNPLCGSTQHWSEWSHLIHWGSNTSKENPFLFALEAHHHHHH

SEQ ID NO: 213 - protein, 501 aa, synthetic construct
MLKPSLPFRSLLVLQLPLLGVALNTTILTPNGNEDATTDFFLTSMPTDSLSVSTLPLPEVQCFVFNVEYMNCTWNSSSEPQPTNLTLHYWYKNSDNDKVQKCSHYLFSEEITSGCQLQEKEIHLYQTFVVQLQDPREPRRQATQMLKLQNLVIPWAPENLTLRKLSESQLELNWNNRFLNHCLEHLVQYRTDWDHSWTEQSVDYRHKFSLPSVDGQKRYTFRVRSRFNPLCGSAQHWSEWSHPIHWGSNSSKENGGGGSGGGGSGGGGSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKTISRSLGK

SEQ ID NO: 214 - protein, 260 aa, synthetic construct
MLKPSLPFRSLLVLQLPLLGVALNTTILTPNGNEDATTDFFLTSMPTDSLSVSTLPLPEVQCFVFNVEYMNCTWNSSSEPQPTNLTLHYWYKNSDNDKVQKCSHYLFSEEITSGCQLQEKEIHLYQTFVVQLQDPREPRRQATQMLKLQNLVIPWAPENLTLRKLSESQLELNWNNRFLNHCLEHLVQYRTDWDHSWTEQSVDYRHKFSLPSVDGQKRYTFRVRSRFNPLCGSAQHWSEWSHPIHWGSNSSKENHHHHHH

SEQ ID NO: 215 - protein, 500 aa, synthetic construct
MLKPPLPLRSLLFLQLPLLGVGLNQRILTPSENEDTTDSFLTSLSPGSLSVSTLPLPEVQCFVYNVEYMNCTWNSSSESQPTNLTLHYRYENSDNYKFQECGYYIFSEEITSGCWLPKEKISLYQTFVVQLQDPREPRRQTTRKLKLQNLVIPWAPENLTLLNLSESQLELSWTNRYLNHCLEHLVQYRSNWDRSWTEQPVHHRQSFSLPSVDGQKLYTFRVRSRFNPLCGSAQHWSEWSHPIHWGSKTSKENGGGGSGGGGSGGGGSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKTISRSLGK

SEQ ID NO: 216 - protein, 259 aa, synthetic construct
MLKPPLPLRSLLFLQLPLLGVGLNQRILTPSENEDTTDSFLTSLSPGSLSVSTLPLPEVQCFVYNVEYMNCTWNSSSESQPTNLTLHYRYENSDNYKFQECGYYIFSEEITSGCWLPKEKISLYQTFVVQLQDPREPRRQTTRKLKLQNLVIPWAPENLTLLNLSESQLELSWTNRYLNHCLEHLVQYRSNWDRSWTEQPVHHRQSFSLPSVDGQKLYTFRVRSRFNPLCGSAQHWSEWSHPIHWGSKTSKENHHHHHH

SEQ ID NO: 217 - protein, 510 aa, synthetic construct
MLKLLLSPRSFLVLQLLLLRAGWSSKVLMSSANEDIKADLILTSTAPEHLSAPTLPLPEVQCFVFNIEYMNCTWNSSSEPQATNLTLHYRYKVSDNNTFQECSHYLFSKEITSGCQIQKEDIQLYQTFVVQLQDPQKPQRRAVQKLNLQNLVIPRAPENLTLSNLSESQLELRWKSRHIKERCLQYLVQYRSNRDRSWTELIVNHEPRFSLPSVDELKRYTFRVRSRYNPICGSSQQWSKWSQPVHWGSHTVEENPSLFALEAGGGGSGGGGSGGGGSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKTISRSLGK

SEQ ID NO: 218 - protein, 269 aa, synthetic construct
MLKLLLSPRSFLVLQLLLLRAGWSSKVLMSSANEDIKADLILTSTAPEHLSAPTLPLPEVQCFVFNIEYMNCTWNSSSEPQATNLTLHYRYKVSDNNTFQECSHYLFSKEITSGCQIQKEDIQLYQTFVVQLQDPQKPQRRAVQKLNLQNLVIPRAPENLTLSNLSESQLELRWKSRHIKERCLQYLVQYRSNRDRSWTELIVNHEPRFSLPSVDELKRYTFRVRSRYNPICGSSQQWSKWSQPVHWGSHTVEENPSLFALEAHHHHHH

SEQ ID NO: 219 - protein, 509 aa, synthetic construct
MLKPLLPSRSFLLLQLLLLRVGWSSKVLMSSGNEDTKSDLLLTSMDLKHLSVPTLPLPEVQCFVFNVEYMNCTWNSSSEPQPTNLTMHYRYKGSDNNTFQECSHYLFSKEITSGCQIQKEDIQLYQTFVVQLQDPQKPQRRAEQKLNLQNLVIPWAPENLTLYNLSESQVELRWKSRYIERCLQYLVQYRSNRDRSWTEQIVDHEPRFSLPSVDEQKLYTFRVRSRFNPICGSTQQWSKWSQPIHWGSHTAEENPSLFALEAGGGGSGGGGSGGGGSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKTISRSLGK

SEQ ID NO: 220 - protein, 268 aa, synthetic construct
MLKPLLPSRSFLLLQLLLLRVGWSSKVLMSSGNEDTKSDLLLTSMDLKHLSVPTLPLPEVQCFVFNVEYMNCTWNSSSEPQPTNLTMHYRYKGSDNNTFQECSHYLFSKEITSGCQIQKEDIQLYQTFVVQLQDPQKPQRRAEQKLNLQNLVIPWAPENLTLYNLSESQVELRWKSRYIERCLQYLVQYRSNRDRSWTEQIVDHEPRFSLPSVDEQKLYTFRVRSRFNPICGSTQQWSKWSQPIHWGSHTAEENPSLFALEAHHHHHH

SEQ ID NO: 221 - protein, 30 aa, synthetic construct
QVQLVQSGAEVLKPGASVKVSCKASGYTFT

SEQ ID NO: 222 - protein, 14 aa, synthetic construct
WVRQARGQRLEWIG

SEQ ID NO: 223 - protein, 14 aa, synthetic construct
WVKQARGQRLEWIG

SEQ ID NO: 224 - protein, 32 aa, synthetic construct
RVTMTTDTSTSTAYMELRNLRSDDTAVYYCAR

SEQ ID NO: 225 - protein, 32 aa, synthetic construct
KATLTTDTSTSTAYMELRNLRSDDTAVYYCAR

SEQ ID NO: 226 - protein, 11 aa, synthetic construct
WGQGTLVTVSS

SEQ ID NO: 227 - protein, 23 aa, synthetic construct
DIVMTQSPLSLPVTPGEPASISC

SEQ ID NO: 228 - protein, 23 aa, synthetic construct
DIVMTQSPLSLPVTPGEQASISC

SEQ ID NO: 229 - protein, 15 aa, synthetic construct
WYLQKPGQSPQLLIY

SEQ ID NO: 230 - protein, 32 aa, synthetic construct
GVPDRFSGSGSGTDFTLKISRVEAEDVGVYYC

SEQ ID NO: 231 - protein, 32 aa, synthetic construct
GVPDRFSGSGSGTDFTLKISRVEAEDVGVYFC

SEQ ID NO: 232 - protein, 11 aa, synthetic construct
FGQGTKLEIKR

SEQ ID NO: 233 - protein, 11 aa, synthetic construct
FGSGTKLEIKR

SEQ ID NO: 234 - protein, 6 aa, synthetic construct
HHHHHH

SEQ ID NO: 235 - protein, 5 aa, synthetic construct
  VARIANT 3: note = Any amino acid
WSXWS

SEQ ID NO: 236 - protein, 5 aa, synthetic construct
GGGGS

SEQ ID NO: 237 - protein, 15 aa, synthetic construct
GGGGSGGGGSGGGGS